SEQUENCE LISTING (127 sequences)

SEQ ID NO: 1 - DNA, 5350 bp, synthetic construct
  misc_feature 1..5350: note = source = /note="Description of Artificial Sequence: Synthetic polynucleotide"
  source 1..5350: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaagagccaagagggcaccggtgaaacagactttgaattttgaccttctgaagttggcaggagacgttgagtccaaccctgggcccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 2 - DNA, 5350 bp, synthetic construct
  misc_feature 1..5350: note = source = /note="Description of Artificial Sequence: Synthetic polynucleotide"
  source 1..5350: mol_type = other DNA
ctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaagagccaagagggcaccggtgaaacagactttgaattttgaccttctgaagttggcaggagacgttgagtccaaccctgggcccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 3 - DNA, 5641 bp, synthetic construct
  misc_feature 1..5641: note = source = /note="Description of Artificial Sequence: Synthetic polynucleotide"
  source 1..5641: mol_type = other DNA
ctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaagagccaagagggcaccggtgaaacagactttgaattttgaccttctgaagttggcaggagacgttgagtccaaccctgggcccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 4 - DNA, 5641 bp, synthetic construct
  misc_feature 1..5641: note = source = /note="Description of Artificial Sequence: Synthetic polynucleotide"
  source 1..5641: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaagagccaagagggcaccggtgaaacagactttgaattttgaccttctgaagttggcaggagacgttgagtccaaccctgggcccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 5 - DNA, 5332 bp, synthetic construct
  misc_feature 1..5332: note = source = /note="Description of Artificial Sequence: Synthetic polynucleotide"
  source 1..5332: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 6 - DNA, 5332 bp, synthetic construct
  misc_feature 1..5332: note = source = /note="Description of Artificial Sequence: Synthetic polynucleotide"
  source 1..5332: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 7 - DNA, 5623 bp, synthetic construct
  misc_feature 1..5623: note = source = /note="Description of Artificial Sequence: Synthetic polynucleotide"
  source 1..5623: mol_type = other DNA
ctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 8 - DNA, 5623 bp, synthetic construct
  misc_feature 1..5623: note = source = /note="Description of Artificial Sequence: Synthetic polynucleotide"
  source 1..5623: mol_type = other DNA
ctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 9 - DNA, 5359 bp, synthetic construct
  misc_feature 1..5359: note = source = /note="Description of Artificial Sequence: Synthetic polynucleotide"
  source 1..5359: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcacgtgcaaagcgtgcaccggtgaaacagggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 10 - DNA, 5359 bp, synthetic construct
  misc_feature 1..5359: note = source = /note="Description of Artificial Sequence: Synthetic polynucleotide"
  source 1..5359: mol_type = other DNA
ctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaacgtgcaaagcgtgcaccggtgaaacagggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 11 - DNA, 5650 bp, synthetic construct
  misc_feature 1..5650: note = source = /note="Description of Artificial Sequence: Synthetic polynucleotide"
  source 1..5650: mol_type = other DNA
ctagcatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaacgtgcaaagcgtgcaccggtgaaacagggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 12 - DNA, 5650 bp, synthetic construct
  misc_feature 1..5650: note = source = /note="Description of Artificial Sequence: Synthetic polynucleotide"
  source 1..5650: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagacgtgcaaagcgtgcaccggtgaaacagggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 13 - DNA, 5665 bp, synthetic construct
  misc_feature 1..5665: note = source = /note="Description of Artificial Sequence: Synthetic polynucleotide"
  source 1..5665: mol_type = other DNA
ctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagacgtgcaaagcgtgcaccggtgaaacagggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 14 - DNA, 5656 bp, synthetic construct
  misc_feature 1..5656: note = source = /note="Description of Artificial Sequence: Synthetic polynucleotide"
  source 1..5656: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaagagccaagagggcaccggtgaaacagactttgaattttgaccttctgaagttggcaggagacgttgagtccaaccctgggcccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 15 - DNA, 5656 bp, synthetic construct
  misc_feature 1..5656: note = source = /note="Description of Artificial Sequence: Synthetic polynucleotide"
  source 1..5656: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaagagccaagagggcaccggtgaaacagactttgaattttgaccttctgaagttggcaggagacgttgagtccaaccctgggcccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 16 - DNA, 5638 bp, synthetic construct
  misc_feature 1..5638: note = source = /note="Description of Artificial Sequence: Synthetic polynucleotide"
  source 1..5638: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 17 - DNA, 5638 bp, synthetic construct
  misc_feature 1..5638: note = source = /note="Description of Artificial Sequence: Synthetic polynucleotide"
  source 1..5638: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 18 - DNA, 5665 bp, synthetic construct
  misc_feature 1..5665: note = source = /note="Description of Artificial Sequence: Synthetic polynucleotide"
  source 1..5665: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcacgtgcaaagcgtgcaccggtgaaacagggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 19 - DNA, 5356 bp, synthetic construct
  misc_feature 1..5356: note = source = /note="Description of Artificial Sequence: Synthetic polynucleotide"
  source 1..5356: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaagagccaagagggcaccggtgaaacagactttgaattttgaccttctgaagttggcaggagacgttgagtccaaccctgggcccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 20 - DNA, 5356 bp, synthetic construct
  misc_feature 1..5356: note = source = /note="Description of Artificial Sequence: Synthetic polynucleotide"
  source 1..5356: mol_type = other DNA
ctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaagagccaagagggcaccggtgaaacagactttgaattttgaccttctgaagttggcaggagacgttgagtccaaccctgggcccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 21 - DNA, 5647 bp, synthetic construct
  misc_feature 1..5647: note = source = /note="Description of Artificial Sequence: Synthetic polynucleotide"
  source 1..5647: mol_type = other DNA
ctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaagagccaagagggcaccggtgaaacagactttgaattttgaccttctgaagttggcaggagacgttgagtccaaccctgggcccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 22 - DNA, 5647 bp, synthetic construct
  misc_feature 1..5647: note = source = /note="Description of Artificial Sequence: Synthetic polynucleotide"
  source 1..5647: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaagagccaagagggcaccggtgaaacagactttgaattttgaccttctgaagttggcaggagacgttgagtccaaccctgggcccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 23 - DNA, 5338 bp, synthetic construct
  misc_feature 1..5338: note = source = /note="Description of Artificial Sequence: Synthetic polynucleotide"
  source 1..5338: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcaggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 24 - DNA, 5338 bp, synthetic construct
  misc_feature 1..5338: note = source = /note="Description of Artificial Sequence: Synthetic polynucleotide"
  source 1..5338: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 25 - DNA, 5629 bp, synthetic construct
  misc_feature 1..5629: note = source = /note="Description of Artificial Sequence: Synthetic polynucleotide"
  source 1..5629: mol_type = other DNA
ctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaaggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 26 - DNA, 5629 bp, synthetic construct
  misc_feature 1..5629: note = source = /note="Description of Artificial Sequence: Synthetic polynucleotide"
  source 1..5629: mol_type = other DNA
ctagcgccaccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagaggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 27 - DNA, 5365 bp, synthetic construct
  misc_feature 1..5365: note = source = /note="Description of Artificial Sequence: Synthetic polynucleotide"
  source 1..5365: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcacgtgcaaagcgtgcaccggtgaaacagggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 28 - DNA, 5365 bp, synthetic construct
  misc_feature 1..5365: note = source = /note="Description of Artificial Sequence: Synthetic polynucleotide"
  source 1..5365: mol_type = other DNA
ctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaacgtgcaaagcgtgcaccggtgaaacagggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 29 - DNA, 5656 bp, synthetic construct
  misc_feature 1..5656: note = source = /note="Description of Artificial Sequence: Synthetic polynucleotide"
  source 1..5656: mol_type = other DNA
ctagcgccaccatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaacgtgcaaagcgtgcaccggtgaaacagggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 30 - DNA, 5656 bp, synthetic construct
  misc_feature 1..5656: note = source = /note="Description of Artificial Sequence: Synthetic polynucleotide"
  source 1..5656: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcgccaccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagacgtgcaaagcgtgcaccggtgaaacagggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatcgatccaaagcttaagtggatccaggagtatctggaaaaggctctcaataaataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 31 - DNA, 5671 bp, synthetic construct
  misc_feature 1..5671: note = source = /note="Description of Artificial Sequence: Synthetic polynucleotide"
  source 1..5671: mol_type = other DNA
ctagcgccaccatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagacgtgcaaagcgtgcaccggtgaaacagggaagcggagctactaacttcagcctgctgaagcaggctggagacgtggaggagaaccctggacctatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaataactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccg

SEQ ID NO: 32 - protein, 4 aa, unidentified
  REGION 1..4: note = source = /note="Description of Unknown: Furinlink1 linker"
RAKR

SEQ ID NO: 33 - DNA, 12 bp, unidentified
  misc_feature 1..12: note = source = /note="Description of Unknown: Furinlink1 linker"
  source 1..12: mol_type = other DNA
cgtgcaaagcgt

SEQ ID NO: 34 - protein, 28 aa, unidentified
  REGION 1..28: note = source = /note="Description of Unknown: fmdv linker"
RAKRAPVKQTLNFDLLKLAGDVESNPGP

SEQ ID NO: 35 - DNA, 84 bp, unidentified
  misc_feature 1..84: note = source = /note="Description of Unknown: fmdv linker"
  source 1..84: mol_type = other DNA
agagccaagagggcaccggtgaaacagactttgaattttgaccttctgaagttggcaggagacgttgagtccaaccctgggccc

SEQ ID NO: 36 - protein, 19 aa, unidentified
  REGION 1..19: note = source = /note="Description of Unknown: p2a linker"
ATNFSLLKQAGDVEENPGP

SEQ ID NO: 37 - DNA, 57 bp, unidentified
  misc_feature 1..57: note = source = /note="Description of Unknown: p2a linker"
  source 1..57: mol_type = other DNA
gctactaacttcagcctgctgaagcaggctggagacgtggaggagaaccctggacct

SEQ ID NO: 38 - protein, 22 aa, unidentified
  REGION 1..22: note = source = /note="Description of Unknown: GSG-p2a linker"
GSGATNFSLLKQAGDVEENPGP

SEQ ID NO: 39 - DNA, 66 bp, unidentified
  misc_feature 1..66: note = source = /note="Description of Unknown: GSG-p2a linker"
  source 1..66: mol_type = other DNA
ggaagcggagctactaacttcagcctgctgaagcaggctggagacgtggaggagaaccctggacct

SEQ ID NO: 40 - protein, 31 aa, unidentified
  REGION 1..31: note = source = /note="Description of Unknown: fp2a linker"
RAKRAPVKQGSGATNFSLLKQAGDVEENPGP

SEQ ID NO: 41 - DNA, 93 bp, unidentified
  misc_feature 1..93: note = source = /note="Description of Unknown: fp2a linker"
  source 1..93: mol_type = other DNA
cgtgcaaagcgtgcaccggtgaaacagggaagcggagctactaacttcagcctgctgaagcaggctggagacgtggaggagaaccctggacct

SEQ ID NO: 42 - protein, 5 aa, synthetic construct
  REGION 1..5: note = source = /note="Description of Artificial Sequence: Synthetic peptide"
APVKQ

SEQ ID NO: 43 - protein, 5 aa, synthetic construct
  REGION 1..5: note = source = /note="Description of Artificial Sequence: Synthetic peptide"
GPVKQ

SEQ ID NO: 44 - protein, 5 aa, synthetic construct
  REGION 1..5: note = source = /note="Description of Artificial Sequence: Synthetic peptide"
VPVKQ

SEQ ID NO: 45 - protein, 5 aa, synthetic construct
  REGION 1..5: note = source = /note="Description of Artificial Sequence: Synthetic peptide"
IPVKQ

SEQ ID NO: 46 - protein, 5 aa, synthetic construct
  REGION 1..5: note = source = /note="Description of Artificial Sequence: Synthetic peptide"
MPVKQ

SEQ ID NO: 47 - protein, 5 aa, synthetic construct
  REGION 1..5: note = source = /note="Description of Artificial Sequence: Synthetic peptide"
APIKQ

SEQ ID NO: 48 - protein, 5 aa, synthetic construct
  REGION 1..5: note = source = /note="Description of Artificial Sequence: Synthetic peptide"
GPIKQ

SEQ ID NO: 49 - protein, 5 aa, synthetic construct
  REGION 1..5: note = source = /note="Description of Artificial Sequence: Synthetic peptide"
VPIKQ

SEQ ID NO: 50 - protein, 5 aa, synthetic construct
  REGION 1..5: note = source = /note="Description of Artificial Sequence: Synthetic peptide"
IPIKQ

SEQ ID NO: 51 - protein, 5 aa, synthetic construct
  REGION 1..5: note = source = /note="Description of Artificial Sequence: Synthetic peptide"
MPIKQ

SEQ ID NO: 52 - protein, 5 aa, synthetic construct
  REGION 1..5: note = source = /note="Description of Artificial Sequence: Synthetic peptide"
APAKQ

SEQ ID NO: 53 - protein, 5 aa, synthetic construct
  REGION 1..5: note = source = /note="Description of Artificial Sequence: Synthetic peptide"
GPAKQ

SEQ ID NO: 54 - protein, 5 aa, synthetic construct
  REGION 1..5: note = source = /note="Description of Artificial Sequence: Synthetic peptide"
VPAKQ

SEQ ID NO: 55 - protein, 5 aa, synthetic construct
  REGION 1..5: note = source = /note="Description of Artificial Sequence: Synthetic peptide"
IPAKQ

SEQ ID NO: 56 - protein, 5 aa, synthetic construct
  REGION 1..5: note = source = /note="Description of Artificial Sequence: Synthetic peptide"
MPAKQ

SEQ ID NO: 57 - protein, 5 aa, synthetic construct
  REGION 1..5: note = source = /note="Description of Artificial Sequence: Synthetic peptide"
APVRQ

SEQ ID NO: 58 - protein, 5 aa, synthetic construct
  REGION 1..5: note = source = /note="Description of Artificial Sequence: Synthetic peptide"
GPVRQ

SEQ ID NO: 59 - protein, 5 aa, synthetic construct
  REGION 1..5: note = source = /note="Description of Artificial Sequence: Synthetic peptide"
VPVRQ

SEQ ID NO: 60 - protein, 5 aa, synthetic construct
  REGION 1..5: note = source = /note="Description of Artificial Sequence: Synthetic peptide"
IPVRQ

SEQ ID NO: 61 - protein, 5 aa, synthetic construct
  REGION 1..5: note = source = /note="Description of Artificial Sequence: Synthetic peptide"
MPVRQ

SEQ ID NO: 62 - protein, 5 aa, synthetic construct
  REGION 1..5: note = source = /note="Description of Artificial Sequence: Synthetic peptide"
APIRQ

SEQ ID NO: 63 - protein, 5 aa, synthetic construct
  REGION 1..5: note = source = /note="Description of Artificial Sequence: Synthetic peptide"
GPIRQ

SEQ ID NO: 64 - protein, 5 aa, synthetic construct
  REGION 1..5: note = source = /note="Description of Artificial Sequence: Synthetic peptide"
VPIRQ

SEQ ID NO: 65 - protein, 5 aa, synthetic construct
  REGION 1..5: note = source = /note="Description of Artificial Sequence: Synthetic peptide"
IPIRQ

SEQ ID NO: 66 - protein, 5 aa, synthetic construct
  REGION 1..5: note = source = /note="Description of Artificial Sequence: Synthetic peptide"
MPIRQ

SEQ ID NO: 67 - protein, 5 aa, synthetic construct
  REGION 1..5: note = source = /note="Description of Artificial Sequence: Synthetic peptide"
APARQ

SEQ ID NO: 68 - protein, 5 aa, synthetic construct
  REGION 1..5: note = source = /note="Description of Artificial Sequence: Synthetic peptide"
GPARQ

SEQ ID NO: 69 - protein, 5 aa, synthetic construct
  REGION 1..5: note = source = /note="Description of Artificial Sequence: Synthetic peptide"
VPARQ

SEQ ID NO: 70 - protein, 5 aa, synthetic construct
  REGION 1..5: note = source = /note="Description of Artificial Sequence: Synthetic peptide"
IPARQ

SEQ ID NO: 71 - protein, 5 aa, synthetic construct
  REGION 1..5: note = source = /note="Description of Artificial Sequence: Synthetic peptide"
MPARQ

SEQ ID NO: 72 - protein, 5 aa, synthetic construct
  REGION 1..5: note = source = /note="Description of Artificial Sequence: Synthetic peptide"
APVKN

SEQ ID NO: 73 - protein, 5 aa, synthetic construct
  REGION 1..5: note = source = /note="Description of Artificial Sequence: Synthetic peptide"
GPVKN

SEQ ID NO: 74 - protein, 5 aa, synthetic construct
  REGION 1..5: note = source = /note="Description of Artificial Sequence: Synthetic peptide"
VPVKN

SEQ ID NO: 75 - protein, 5 aa, synthetic construct
  REGION 1..5: note = source = /note="Description of Artificial Sequence: Synthetic peptide"
IPVKN

SEQ ID NO: 76 - protein, 5 aa, synthetic construct
  REGION 1..5: note = source = /note="Description of Artificial Sequence: Synthetic peptide"
MPVKN

SEQ ID NO: 77 - protein, 5 aa, synthetic construct
  REGION 1..5: note = source = /note="Description of Artificial Sequence: Synthetic peptide"
APIKN

SEQ ID NO: 78 - protein, 5 aa, synthetic construct
  REGION 1..5: note = source = /note="Description of Artificial Sequence: Synthetic peptide"
GPIKN

SEQ ID NO: 79 - protein, 5 aa, synthetic construct
  REGION 1..5: note = source = /note="Description of Artificial Sequence: Synthetic peptide"
VPIKN

SEQ ID NO: 80 - protein, 5 aa, synthetic construct
  REGION 1..5: note = source = /note="Description of Artificial Sequence: Synthetic peptide"
IPIKN

SEQ ID NO: 81 - protein, 5 aa, synthetic construct
  REGION 1..5: note = source = /note="Description of Artificial Sequence: Synthetic peptide"
MPIKN

SEQ ID NO: 82 - protein, 5 aa, synthetic construct
  REGION 1..5: note = source = /note="Description of Artificial Sequence: Synthetic peptide"
APAKN

SEQ ID NO: 83 - protein, 5 aa, synthetic construct
  REGION 1..5: note = source = /note="Description of Artificial Sequence: Synthetic peptide"
GPAKN

SEQ ID NO: 84 - protein, 5 aa, synthetic construct
  REGION 1..5: note = source = /note="Description of Artificial Sequence: Synthetic peptide"
VPAKN

SEQ ID NO: 85 - protein, 5 aa, synthetic construct
  REGION 1..5: note = source = /note="Description of Artificial Sequence: Synthetic peptide"
IPAKN

SEQ ID NO: 86 - protein, 5 aa, synthetic construct
  REGION 1..5: note = source = /note="Description of Artificial Sequence: Synthetic peptide"
MPAKN

SEQ ID NO: 87 - protein, 5 aa, synthetic construct
  REGION 1..5: note = source = /note="Description of Artificial Sequence: Synthetic peptide"
APVRN

SEQ ID NO: 88 - protein, 5 aa, synthetic construct
  REGION 1..5: note = source = /note="Description of Artificial Sequence: Synthetic peptide"
GPVRN

SEQ ID NO: 89 - protein, 5 aa, synthetic construct
  REGION 1..5: note = source = /note="Description of Artificial Sequence: Synthetic peptide"
VPVRN

SEQ ID NO: 90 - protein, 5 aa, synthetic construct
  REGION 1..5: note = source = /note="Description of Artificial Sequence: Synthetic peptide"
IPVRN

SEQ ID NO: 91 - protein, 5 aa, synthetic construct
  REGION 1..5: note = source = /note="Description of Artificial Sequence: Synthetic peptide"
MPVRN

SEQ ID NO: 92 - protein, 5 aa, synthetic construct
  REGION 1..5: note = source = /note="Description of Artificial Sequence: Synthetic peptide"
APIRN

SEQ ID NO: 93 - protein, 5 aa, synthetic construct
  REGION 1..5: note = source = /note="Description of Artificial Sequence: Synthetic peptide"
GPIRN

SEQ ID NO: 94 - protein, 5 aa, synthetic construct
  REGION 1..5: note = source = /note="Description of Artificial Sequence: Synthetic peptide"
VPIRN

SEQ ID NO: 95 - protein, 5 aa, synthetic construct
  REGION 1..5: note = source = /note="Description of Artificial Sequence: Synthetic peptide"
IPIRN

SEQ ID NO: 96 - protein, 5 aa, synthetic construct
  REGION 1..5: note = source = /note="Description of Artificial Sequence: Synthetic peptide"
MPIRN

SEQ ID NO: 97 - protein, 5 aa, synthetic construct
  REGION 1..5: note = source = /note="Description of Artificial Sequence: Synthetic peptide"
APARN

SEQ ID NO: 98 - protein, 5 aa, synthetic construct
  REGION 1..5: note = source = /note="Description of Artificial Sequence: Synthetic peptide"
GPARN

SEQ ID NO: 99 - protein, 5 aa, synthetic construct
  REGION 1..5: note = source = /note="Description of Artificial Sequence: Synthetic peptide"
VPARN

SEQ ID NO: 100 - protein, 5 aa, synthetic construct
  REGION 1..5: note = source = /note="Description of Artificial Sequence: Synthetic peptide"
IPARN

SEQ ID NO: 101 - protein, 5 aa, synthetic construct
  REGION 1..5: note = source = /note="Description of Artificial Sequence: Synthetic peptide"
MPARN

SEQ ID NO: 102 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 103 - DNA, 267 bp, Homo sapiens
  source 1..267: mol_type = other DNA
atgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatagatccaaagcttaagtggatccaggagtatctggaaaaggctctcaataaa

SEQ ID NO: 104 - protein, 94 aa, Homo sapiens
MGSELETAMETLINVFHAHSGKEGDKYKLSKKELKELLQTELSGFLDAQKDVDAVDKVMKELDENGDGEVDFQEYVVLVAALTVACNNFFWENS

SEQ ID NO: 105 - DNA, 282 bp, Homo sapiens
  source 1..282: mol_type = other DNA
atgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattca

SEQ ID NO: 106 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 107 - DNA, 573 bp, Homo sapiens
  source 1..573: mol_type = other DNA
atgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaaga

SEQ ID NO: 108 - DNA, 6034 bp, synthetic construct
  misc_feature 1..6034: note = source = /note="Description of Artificial Sequence: Synthetic polynucleotide"
  source 1..6034: mol_type = other DNA
taactataacggtcctaaggtagcgacgtacgaaccgttgggcgcgcctggggatagcgatcgctgctggcgcggtccgctatgaggtctctgatagaccacagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccctgcaggaaattgagcccgcagcctcccgcttcgctctctgctcctcctgttcgacagtcagccgcatcttcttttgcgtcgccagccgagccacatcgctcagacaccgctagcatgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcacgtgcaaagcgtgcaccggtgaaacagggaagcggagctactaacttcagcctgctgaagcaggctggagacgtggaggagaaccctggacctatgaatgccaaggtcgttgtggtgcttgtacttgtgctgactgctctgtgtctgagcgacggaaaaccagtctccctcagctacaggtgcccatgccgattcttcgaatctcatgtggcccgggccaatgtgaagcacttgaaaatcctgaatacacccaactgcgcgttgcagatcgtggcccgcctgaaaaataataataggcaggtatgtatagatccaaagcttaagtggatccaggagtatctggaaaaggctctcaataaacgtgcaaagcgtgcaccggtgaaacagggaagcggagctactaacttcagcctgctgaagcaggctggagacgtggaggagaaccctggacctatgaattttctgctctcttgggtgcactggtcactggcactgctgctgtatctgcaccatgcaaaatggtcccaagcagctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaagataaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaaggctcgagctggcggaataggttccaatctgtcgcggccgcattaccctgttatccctaatctcgtttaactatgactctcttaaggtagccaaattccggaactataaattgcgttgcgctcactgcccgctttccagtcgggaaacctgtcgtgccagctgcataaatgaatcggccaacgcgcggggagaggcggtttgcgtattgggcgcgcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacatgcgcatctgacgctcagtggaacgaaaactcacgttaagggattttggtcatgcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcattcatttatcagggttattgtctcatgagcggatacatatttgaatgtatttaggctgagcatctatgtcgggtgcggagaaagaggtaatgaaatggcaggcgcctttttcgttagatatgtagtaagtatcttaatatacagctttatctgttttttaagatacttactacttttcttagtggaaactattagtggctgttaattaagctagtactacccaagatttgacagaatgcatcgtttgcattcgaa

SEQ ID NO: 109 - protein, 30 aa, synthetic construct
  REGION 1..30: note = MISC_FEATURE - /note="This sequence may encompass 0-15 "Gly Ser" repeating units wherein some positions may be absent"
  REGION 1..30: note = source = /note="Description of Artificial Sequence: Synthetic polypeptide"
GSGSGSGSGSGSGSGSGSGSGSGSGSGSGS

SEQ ID NO: 110 - protein, 30 aa, synthetic construct
  REGION 1..30: note = MISC_FEATURE - /note="This sequence may encompass 0-15 "Ser Gly" repeating units wherein some positions may be absent"
  REGION 1..30: note = source = /note="Description of Artificial Sequence: Synthetic polypeptide"
SGSGSGSGSGSGSGSGSGSGSGSGSGSGSG

SEQ ID NO: 111 - protein, 45 aa, synthetic construct
  REGION 1..45: note = MISC_FEATURE - /note="This sequence may encompass 0-15 "Gly Ser Gly" repeating units wherein some positions may be absent"
  REGION 1..45: note = source = /note="Description of Artificial Sequence: Synthetic polypeptide"
GSGGSGGSGGSGGSGGSGGSGGSGGSGGSGGSGGSGGSGGSGGSG

SEQ ID NO: 112 - protein, 45 aa, synthetic construct
  REGION 1..45: note = MISC_FEATURE - /note="This sequence may encompass 0-15 "Ser Gly Ser" repeating units wherein some positions may be absent"
  REGION 1..45: note = source = /note="Description of Artificial Sequence: Synthetic polypeptide"
SGSSGSSGSSGSSGSSGSSGSSGSSGSSGSSGSSGSSGSSGSSGS

SEQ ID NO: 113 - protein, 30 aa, synthetic construct; identical to SEQ ID NO: 109
  REGION 1..30: note = MISC_FEATURE - /note="This sequence may encompass 1-15 "Gly Ser" repeating units wherein some positions may be absent"
  REGION 1..30: note = source = /note="Description of Artificial Sequence: Synthetic polypeptide"

SEQ ID NO: 114 - protein, 30 aa, synthetic construct; identical to SEQ ID NO: 110
  REGION 1..30: note = MISC_FEATURE - /note="This sequence may encompass 1-15 "Ser Gly" repeating units wherein some positions may be absent"
  REGION 1..30: note = source = /note="Description of Artificial Sequence: Synthetic polypeptide"

SEQ ID NO: 115 - protein, 45 aa, synthetic construct; identical to SEQ ID NO: 111
  REGION 1..45: note = MISC_FEATURE - /note="This sequence may encompass 1-15 "Gly Ser Gly" repeating units wherein some positions may be absent"
  REGION 1..45: note = source = /note="Description of Artificial Sequence: Synthetic polypeptide"

SEQ ID NO: 116 - protein, 45 aa, synthetic construct; identical to SEQ ID NO: 112
  REGION 1..45: note = MISC_FEATURE - /note="This sequence may encompass 1-15 "Ser Gly Ser" repeating units wherein some positions may be absent"
  REGION 1..45: note = source = /note="Description of Artificial Sequence: Synthetic polypeptide"

SEQ ID NO: 117 - protein, 8 aa, synthetic construct
  VARIANT 2: note = /replace="Ile"
  SITE 2: note = MISC_FEATURE - /note="Variant residue given in the sequence has no preference with respect to that in the annotations for variant position"
  MOD_RES 4: note = Any amino acid
  REGION 1..8: note = source = /note="Description of Artificial Sequence: Synthetic peptide"
DVEXNPGP

SEQ ID NO: 118 - protein, 4 aa, synthetic construct
  REGION 1..4: note = source = /note="Description of Artificial Sequence: Synthetic peptide"
SGSG

SEQ ID NO: 119 - protein, 8 aa, synthetic construct
  REGION 1..8: note = source = /note="Description of Artificial Sequence: Synthetic peptide"
APVKQGSG

SEQ ID NO: 120 - protein, 5 aa, synthetic construct
  REGION 1..5: note = source = /note="Description of Artificial Sequence: Synthetic peptide"
GGGGS

SEQ ID NO: 121 - protein, 10 aa, synthetic construct
  MOD_RES 1: note = Any amino acid
  MOD_RES 3: note = Any amino acid
  MOD_RES 5: note = Any amino acid
  MOD_RES 7: note = Any amino acid
  MOD_RES 9: note = Any amino acid
  REGION 1..10: note = MISC_FEATURE - /note="This sequence may encompass 2-5 "Xaa Pro" repeating units wherein some positions may be absent"
  REGION 1..10: note = source = /note="Description of Artificial Sequence: Synthetic peptide"
XPXPXPXPXP

SEQ ID NO: 122 - protein, 27 aa, synthetic construct
  REGION 2..26: note = MISC_FEATURE - /note="This region may encompass 2-5 "Glu Ala Ala Ala Lys" repeating units wherein some positions may be absent"
  REGION 1..27: note = source = /note="Description of Artificial Sequence: Synthetic peptide"
AEAAAKEAAAKEAAAKEAAAKEAAAKA

SEQ ID NO: 123 - protein, 30 aa, synthetic construct
  REGION 1..30: note = source = /note="Description of Artificial Sequence: 2A tail"
RAKRAPVKQGSGATNFSLLKQAGDVEENPG

SEQ ID NO: 124 - protein, 124 aa, synthetic construct
  REGION 1..124: note = source = /note="Description of Artificial Sequence: S100A1 with 2A tail"
MGSELETAMETLINVFHAHSGKEGDKYKLSKKELKELLQTELSGFLDAQKDVDAVDKVMKELDENGDGEVDFQEYVVLVAALTVACNNFFWENSRAKRAPVKQGSGATNFSLLKQAGDVEENPG

SEQ ID NO: 125 - DNA, 372 bp, synthetic construct
  misc_feature 1..372: note = source = /note="Description of Artificial Sequence: Synthetic polynucleotide"
  source 1..372: mol_type = other DNA
atgggcagcgaactggaaaccgccatggagactttgataaatgttttccacgcgcatagcggcaaagaaggggacaagtacaagctgtcaaaaaaggagctgaaagaactgctgcagaccgaattgagcggcttcctggacgctcagaaagatgtcgatgccgtcgacaaagtgatgaaagagcttgacgagaacggtgacggtgaagtcgattttcaggaatatgtggtgctggtggccgcccttactgtagcatgcaacaatttcttttgggaaaattcacgtgcaaagcgtgcaccggtgaaacagggaagcggagctactaacttcagcctgctgaagcaggctggagacgtggaggagaaccctgga

SEQ ID NO: 126 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 127 - DNA, 495 bp, Homo sapiens
  source 1..495: mol_type = other DNA
gctcccatggcagagggaggtggacagaatcatcatgaggttgtcaaatttatggatgtctaccagcggagctactgccacccaattgagacgttggtagacatttttcaggaatatccagacgagattgagtacattttcaagcctagctgtgtgcccttgatgcgatgcggtggctgttgcaatgatgagggactcgagtgtgtccccaccgaggaaagcaatataaccatgcaaatcatgcgaatcaaaccccaccagggccagcatatcggcgagatgtctttcttgcaacataacaaatgcgagtgtcggccaaagaaggacagggctcgccaggaaaatccctgtggtccttgttcagagcgcaggaagcatcttttcgtccaggatccgcagacttgtaaatgttcatgcaagaataccgattctaggtgtaaggcgaggcaactcgagcttaacgagagaacctgtaggtgtgacaaacctagaaga

(19) United States
(12) Patent Application Publication
(10) Pub. No.: US 2024/0148908 A1
(43) Pub. Date: May 9, 2024

(54) Compositions and methods for expression of multiple biologically active polypeptides from a single vector for treatment of cardiac conditions and other pathologies

(71) Applicant: PRECIGEN, INC., Germantown, MD (US)

(72) Inventors: Dimki S. PATEL, Germantown, MD (US); Amit N. PATEL, Germantown, MD (US)

(73) Assignee: PRECIGEN, INC., Germantown, MD (US)

(21) Appl. No.: 18/485,838

(22) Filed: Oct. 12, 2023

Related U.S. Application Data
(62) Division of application No. 17/481,938, filed on Sep. 22, 2021, now abandoned.
Application No. 17/481,938 is a division of application No. 15/978,036, filed on May 11, 2018, now Pat. No. 11,154,622.
Application No. 15/978,036 is a continuation of application No. PCT/US2016/061668, filed on Nov. 11, 2016.
(60) Provisional application No. 62/254,139, filed on Nov. 11, 2015.

(51) Int. Cl. A61K 48/00; A61K 38/17; A61K 38/18; A61K 38/19; A61K 41/00; A61P 9/00; C07K 14/47; C07K 14/475; C07K 14/52; C12N 15/62

(52) U.S. Cl. CPC A61K 48/0058; A61K 38/1738; A61K 38/1866; A61K 38/195; A61K 41/0028; A61K 48/00; A61P 9/00; C07K 14/4728; C07K 14/475; C07K 14/52; C07K 14/521; C12N 15/62; C07K 2319/00; C07K 2319/50

(57) ABSTRACT

The present invention provides compositions and methods useful for treating disorders amenable to therapy via introduction of multigenic expression vectors. More particularly, the invention provides vectors and polynucleotides encoding polypeptides for treatment of cardiac disorders wherein said polypeptides may comprise a cytokine, a chemokine, and/or an angiogenic polypeptide, or functional derivatives thereof. Also provided, as compositions of the invention, are linkers useful for connecting and expressing functional (biologically active) polypeptides from single, multigenic-expression constructs.

### CROSS-REFERENCE TO RELATED APPLICATION

The present application is a divisional application of U.S. patent application Ser. No. 15/978,036, filed May 11, 2018, which is a continuation of PCT International Application No. PCT/US2016/061668, filed on Nov. 11, 2016, which claims the benefit of U.S. provisional Patent Application No. 62/254,139, filed Nov. 11, 2015, each of which is hereby incorporated by reference in its entirety for all purposes.

### REFERENCE TO SEQUENCE LISTING

The present application contains a Sequence Listing which has been filed electronically in ASCII format and is hereby incorporated by reference in its entirety. Said ASCII copy, created on Sep. 21, 2021, is named 50471_701_401_SL.txt and is 271,884 bytes in size.

### FIELD OF THE INVENTION

The present invention provides novel nucleic acids and vectors, and polypeptides encoded by same, for multigenic therapeutic treatment of diseases, disorders and pathologic conditions. More particularly, the present invention provides novel nucleic acids, vectors, polypeptides and methods for multigenic treatment and prevention of cardiac diseases and disorders. Moreover, the present invention provides novel nucleic acids and polypeptide linkers, which provide advantageous protein expression from nucleic acids and vectors, useful for multigenic therapeutic treatment of diseases, disorders and pathologic conditions.

### BACKGROUND OF THE INVENTION

Cardiac disease represents a significant unmet medical need; with some estimates indicating at least 25 million patients worldwide. Moreover, according to the United States of America (U.S.) government's Centers for Disease Control and Prevention (CDC) “Heart Failure Fact Sheet” as of 2013, over 5 million people in the U.S. have heart failure conditions.

Heart failure has been estimated to cause 1 in 9 deaths with as many as 825,000 new cases each year. The average survival rate 5 years after diagnosis is at about 40% and represents the highest hospital readmission rate among any diagnosis-related group. The cost in the U.S. has been estimated to be as high as $32 billion per year. Heart failure treatment options include medications, invasive devices, and heart transplant.

Congestive heart failure (CHF) describes the inability of the heart to provide sufficient cardiac output to supply the metabolic demand of the body. There are more than 22 million people worldwide currently diagnosed with CHF and over 5 million patients in the US. Because the incidence and severity of heart disease increases with age, the overall incidence is expected to rise in the future due to the aging population. The prognosis for patients with CHF remains poor, with a five year mortality rate of 50%. According to the American Heart Association (AHA), cardiovascular disease claimed 810,000 lives in the United States in 2013, which accounts for ˜1 in every 3 reported deaths. Pharmacological management of end stage heart failure focus on three goals as follows: 1) improvement of morbidity and mortality (ACE inhibitors, angiotensin II type I receptor antagonists, selected β-blockers, and aldosterone antagonists); 2) control of symptoms (diuretics (eventually thiazide plus loop diuretic), digitalis (low dose); temporary inotropes, and selected anti-arrhythmics); and 3) palliation (opioids, antidepressants, anxiolytics, oxygen and continuous inotropes). However, as disease progresses, therapeutic options become limited to cardiac resynchronization therapy (CRT); considering implantable cardioverter-defibrillator (ICD); heart transplantation and ventricular assist devices (VAD), which are used both as a bridge to transplantation and increasingly as destination therapy due to the lack of donor hearts. Although the overall 5-year survival is 70-80% in heart transplantation patients receiving triple immunosuppressive therapy, heart transplantation as a treatment option is limited by the continuing shortage of donor hearts, the increasing number of transplant candidates and the very high yearly cost over $100,000 per year. Data collected by the Interagency Registry for Mechanically Assisted Circulatory Support (INTERMACS), showed that between Jun. 23, 2006 and Jun. 30 2013, 12,335 patients received an FDA approved durable mechanical circulatory support (MCS) device, with a rate of accrual that has continued at a pace of 2,000 patients per year.

There is an increasing study of cell and gene therapies for the treatment of CHF, but with limited results due to issues with biologic effect, cell retention, timing of delivery, and lack of mechanism or limited single gene effect. Even so, many clinical gene therapy trials have demonstrated modest effects at one year. Although gene therapy has a defined mechanism of action, single genes used to improve angiogenesis, stem cell homing, or inotropy have not been sufficient to treat CHF. Because CHF is multifactorial in terms of scarring, decreased contractile function and cell loss, a multigenic approach may better address these individual factors while keeping these extremely sick patients safe.

Traditionally, vectors for gene therapy are single gene. An increasing demand for more complex multigene vectors has arisen in recent years. In particular, this demand is stimulated by the need of combination therapies for cancer and antiviral treatment. Combination gene therapy is medicine's best attempt to prevent mutation and resistance in cancer. By combining two or three agents a more complete and effective response may be obtained.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a disclosure herein and a disclosure in an incorporated reference, the disclosure herein controls.

### SUMMARY OF THE DISCLOSURE

An increasing number of patients with congestive heart failure (CHF) continue to have limited therapeutic options. For example, a common CHF treatment results in placement of a destination left ventricular assist device (LVAD)). Accordingly, a different approach involving biologic options to promote recovery from CHF are needed.

A single gene-based approach to treating cardiac disorders is reasonable in patients with unique genetic mutations, i.e. Troponin I or Heavy chain myosin. However, in most patients with cardiac pathology, the end-stage disease is due to multiple factors. Therefore, in such cases a single gene approach is unlikely to work. Cardiac patients usually have scar tissue requiring positive remodeling via cell recruitment and/or angiogenesis. They usually also require an increase in inotropic function via calcium or other pathways. Hence, a combination of genes which effect calcium handling, cell recruitment and angiogenesis together provides a more appropriate path to treat and recover these end-stage CHF patients. Moreover, when considering a gene based approach to treating cardiac disorders (such as, but not limited to, CHF), the use of a viral vectors may antagonize the human immune system, or patients may already have pre-formed anti-viral antibodies (e.g., anti-AAV1/AAV2 antibodies), which could cause safety and efficacy issues. Thus, the use of plasmid DNA, which has low to no adverse immune response in humans and can be re-dosed as needed, provides an advantageous mode of therapeutic gene delivery.

As such, one embodiment of the present invention provides a triple effector non-viral plasmid-based DNA for therapeutic treatment of cardiac diseases and disorders (for example, but not limited to, CHF). In one embodiment, the present invention provides a triple effector non-viral plasmid-based DNA vector (construct). In a particular embodiment of the present invention, an expression plasmid referred to as pXoX encodes and expresses biologically active SDF1α, S100A1, and VEGF, wherein these three effector genes of interest (GOI) are separated by selectively designed linker sequences described herein.

A multigenic plasmid DNA approach to treat cardiac diseases and disorders (for example, but not limited to, CHF) is based on positive in vitro, pre-clinical and clinical data from other studies in which single genes have been utilized. Each gene of interest described below has a specific function for inotropy (e.g., S100A1), cell homing (e.g., SDF-1 α), and angiogenesis (e.g., VEGF165). The rationale for a single plasmid construct comprising all three genes, instead of each individual gene on separately delivered plasmids, is that if single genes (on individual, separate vectors) are delivered to cells, there is no means by which the amount of transfection of each gene to a cell can be controlled. This variability in gene delivery would make the therapy unsafe and non-reproducible even though it would provide a simpler approach than single, multigenic constructs of the present invention. Hence, in one embodiment, vectors such as pXoX constitutively express S100A1 (an intracellular protein), SDF-1α (a secreted protein), and VEGF165 (a secreted protein). With pXoX, the pDNA construct is transcribed as a single mRNA which is translated as three individual functional proteins via cleavage by the combined activity of furin and P2A (fp2a). The 2A self-cleaving peptide encodes a sequence (P2A) that mediates a translational effect known as “ribosome skipping”, “stop-go” and “stop-carry on” translation that results in co-expression of multiple proteins from a single transcript mRNA under the control of a single promoter. Constitutive expression is driven by promoters, such as, but not limited to, a CAG promoter (a hybrid promoter containing the chicken beta-actin (CBA) promoter with a CMV enhancer and hybrid CBA exon 1/rabbit beta-globin intron B). Further description of three effectors and their rationale for inclusion in constructs of the invention are described below.

### S100A1

S100A1 is a member of the Ca2+-binding EF-hand protein superfamily (Rohde et al, J Cardiovasc Transl Res, vol. 3, no. 5, pp. 525-537, 2010). S100A1 is a 10.4 kDa protein that functions as a homodimer primarily in cardiomyocytes to regulate Ca2+-controlled networks and fluxes to control contractile function, excitability, metabolism, maintenance, and survival. Animal and human studies have demonstrated decreased expression of S100A1 in heart failure (Pleger et al., Circulation, vol. 115, no. 19, pp. 2506-2515, 2007; and, Remppis et al., Biochim Biophys Acta, vol. 1313, no. 3, pp. 253-257, 1996). Viral delivery of S100A1 has been successfully used in large animal models of heart failure. Specifically, AAV9-S100A1 gene therapy evaluated in a pig model demonstrated protection from hemodynamic deterioration, improvement of cardiac function, and heart rate normalization (Rohde et al, J Mol Cell Cardiol, vol. 50, no. 5, pp. 777-784, 2011). Another study using AAV6-S100A1 in a pig model of heart failure demonstrated the safety of over-expression of S100A1 demonstrating no increase in arrhythmias or right ventricular dysfunction; however, there was a significant increase in left ventricular ejection fraction and cardiac remodeling (Weber et al, Gene Ther, vol. 21, pp. 131-138, 2014). Studies such as these support the potential for S100A1 therapy in humans with end-stage heart failure.

### SDF-1α

Stromal cell-derived factor-1α (SDF-1α) is an 8 kDa chemokine that plays an important role in recruitment of cardiac stem cells, inhibition of cardiac myocyte death, and improvement in cardiac function by binding to receptor CXCR4 on mesenchymal stem cells (Penn et al, Gene Ther, vol. 19, no. 6, pp. 583-587, 2012). A non-viral DNA plasmid encoding human SDF-1α was tested in several clinical trials (Penn et al., Circ Res, vol. 112, no. 5, pp. 816-825, 2013; and, Chung et al, Eur Heart J, vol. 36, pp. 2228-2238, 2015). Findings from these trials demonstrated that over-expression of SDF-1α is relatively safe with no incidence of serious unanticipated related adverse events including arrhythmias or progression to further heart failure episodes. Such study also demonstrated that SDF-1α over-expression could improve function in patients with CHF injected intramyocardially at doses of 15 mg or 30 mg. Moreover, plasmid DNA delivered retrograde via the coronary sinus at a dose of 45 mg was found to be safe.

### VEGF

The vascular endothelial growth factor (VEGF) family of proteins is involved in new vessel formation, endothelial cell migration and activation, stem cell recruitment, and tissue regeneration (Taimeh et al, Nat Rev Cardiol, vol. 10, no. 9, pp. 519-530, 2013). Dysfunctional vascular regulation is an important component of the pathophysiology of heart failure, and reduced levels of VEGF have been observed in models of advanced heart failure. There are many isoforms of VEGF. However, VEGF165 has been used in both preclinical and clinical models. The utility of targeting VEGF as a treatment option for heart failure has been demonstrated using gene transfer with vectors such as naked plasmid DNA and adenovirus in animal models showing improved collateral perfusion and overall cardiac function. Clinical use of VEGF165 has been studied both as a directly injected pDNA and via a viral vector with positive but limited results. In certain embodiments, VEGF 165 has a sequence comprising amino acids 27-191 of a VEGF191 sequence disclosed herein. In certain embodiments, VEGF 165 has a sequence comprising amino acids 27, 28, 29, 30, 31, 32, 33, or 34 to 180, 181, 182, 183, 184, 185, 186, 187, 188, 189, 190, or 191 of a VEGF191 sequence disclosed herein. In certain embodiments, VEGF 165 has a sequence comprising amino acids 30-185 of a VEGF191 sequence disclosed herein.

The CAG promoter utilized in conjunction with each individual gene (i.e., S100A1, SDF1α, and VEGF165) has demonstrated the individual effects on scar tissue, cardiac function and remodeling. The effect of each gene is unique on cardiac tissue. The potential enhanced effect of all three genes has been tested only in vitro. However, local administration of the combination of VEGF with SDF-1α has been demonstrated to be enhanced in a murine model of hind limb ischemia by promoting endothelial progenitor cell-induced neovascularization (Yu et al, J Vasc Surg, vol. 50, no. 3, pp. 608-616, 2009).

Accordingly, provided herein are polynucleotide constructs (such as, but not limited to, non-viral plasmid vectors) encoding a polypeptide comprising a first functional (i.e., biologically active) polypeptide, a second functional polypeptide and, optionally, a third functional polypeptide.

Provided herein are polynucleotide constructs encoding a polypeptide comprising an S100 polypeptide, a second functional polypeptide and, optionally, a third functional polypeptide, wherein the second and, optional, third functional polypeptides comprise any one of a cytokine, a chemokine, or an angiogenic polypeptide.

Provided herein are polynucleotide constructs comprising polynucleotide sequences encoding polypeptide linker sequences for separation of the first and second and, optionally, separation of the second and third functional polypeptides.

Provided are polypeptides encoded by a polynucleotide described herein. Also provided are cells comprising at least one polynucleotide or vector described herein.

Provided herein are methods of treating a cardiac condition comprising contacting a cell with a therapeutically effective amount of a polynucleotide described herein. In some cases, the cell is a myocardial cell.

Provided herein are methods of treating cardiac diseases and disorders, for example, but not limited to, congestive heart failure (CHF), in a subject comprising providing to a subject a therapeutically effective amount of a composition comprising a polynucleotide or vector described herein. In some cases, the subject is a mammal or human subject. In some cases, the subject is administered at least one additional therapy.

Provided herein are pharmaceutical compositions comprising a polynucleotide described herein, or a polypeptide encoded by a polynucleotide described herein and a pharmaceutically acceptable excipient.

Provided are compositions comprising: at least one plasmid microbubble complex, wherein the microbubble comprises a lipid, a gas and a plasmid comprising a polynucleotide described herein. In some embodiments, the lipid forms a shell enclosing said gas and plasmid. In some cases, the gas is a perfluorocarbon gas, such as perfluoropropane. In some embodiments, the microbubble complex comprises at least one of 1,2-dipalmitoyl-sn-glycero-3-phosphatidylcholine and 1,2-dipalmitoyl-sn-glycero-3-phosphatidylethanolamine glycerol.

Provided are methods comprising administering to a subject a plasmid microbubble complex composition described herein; and contacting the subject with an ultrasonic energy sufficient to result in ultrasound disruption of the at least one microbubble at a predetermined tissue or organ. In some cases, disruption of the at least one microbubble at said predetermined tissue or organ delivers said plasmid into said tissue or organ. In some instances the tissue or organ is an organ, which can be the heart, liver, or kidney.

Provided is a method of treating a cardiac disease or disorder (for example, but not limited to, congestive heart failure, cardiomyopathy, arrhythmia, pericardial disease, aorta disease, marfan syndrome and coronary artery disease.) in a subject comprising: administering to the subject an amount of plasmid microbubble complex composition described herein; contacting the subject with an ultrasonic energy sufficient to result in ultrasound disruption of the at least one microbubble and delivering the plasmid to the heart of the subject.

### BRIEF DESCRIPTION OF THE DRAWINGS

Unique and preferred features of the invention are set forth with particularity in the appended claims. Understanding of the features and advantages of the present invention may also be obtained by reference to the following detailed description that sets forth illustrative embodiments, in which the principles of the invention are utilized, and the accompanying drawings of which:

FIG. 1 displays ways in which non-viral delivery of cardiac effector genes is accomplished via alternate configurations of novel vector constructs comprising linker sequences described herein.

FIG. 2 shows persistent expression of Luciferase in cardiomyocytes from constructs comprising linkers described herein, in primary cells (in vitro) via introduction of vectors comprising att-site-luciferase with SPBc2 (recombinase) transfection. Cells lacking either att-site or recombinase rapidly returned to baseline expression levels. Day 2 (D2), Day 5 (D5), Day 9 (D9), Day 14 (D14).

FIG. 3 shows data for luciferase expression achieved in a rodent model from a plasmid vector backbone from which cardiac effector genes (pXoX encoding biologically active SDF1α, S100A1, and VEGF) may be expressed, via separation by linkers described herein.

FIG. 4 shows various stages of progress through heart failure conditions and the corresponding standard-of-care modes and methods for intervention and treatment.

FIG. 5A and FIG. 5B show a HUVEC proliferation assay. A. HUVEC proliferation following recombinant VEGF treatment B. shows HUVEC proliferation following treatment with VEGF-transfected induced pluripotent stem cells (iPSC) cell supernatant. iPSC cells were transfected and incubated for 96 hours. Supernatant was collected and used to culture HUVECS. HUVEC proliferation was measured by CellTiter-Glo.

FIG. 6 shows HUVEC proliferation following treatment with transfected Cardiomyocyte-iPSC supernatants. Cardiomyocyte-iPSC were obtained from patients with dilated cardiomyopathy (DCM), (hypertrophy cardiomyopathy (HCM) or healthy controls, demonstrating successful expression and activity of functional polypeptides encoded by polynucleotides described herein, comprising linkers disclosed herein.

FIG. 7 shows iPSC-CMs transfected with singe gene, double gene, and triple-gene SDF1 polypeptide constructs showing elevated migration of peripheral blood lymphocytes (PBLs), demonstrating successful expression and activity of functional polypeptides encoded by polynucleotides described herein, comprising linkers disclosed herein.

FIG. 8 shows a migration assay where AMD3100 (CXCR4 antagonist) specifically inhibited SDF1-CXCR4 dependent migration. Data shows that SDF1 is functional in double gene and triple-gene constructs, as migration is reduced in presence of AMD3100.

FIG. 9 shows a porcine study in which bioluminescence images illustrate retrograde perfusion through the coronary sinus of a reporter plasmid, also incorporating a linker described herein, results in delivery to the heart and expression of the delivered reporter gene after 24 hours. The radiance intensity and distribution 24-hours post-retrograde infusion of luciferase-expressing plasmids through the porcine heart coronary sinus. Three plasmid doses (40, 80, and 120 mg) were tested in triplicate.

FIG. 10 shows a migration assay in which cardiomyocytes were transfected with double gene and triple-gene constructs expressing SDF1. Jurkat cell migration using supernatant from the iPSC CMs shows that the transfected and SDF1 protein is functional and produces significant migration, demonstrating successful expression and activity of functional polypeptides encoded by polynucleotides described herein, comprising linkers disclosed herein.

FIG. 11 shows a migration assay in which cardiomyocytes were transfected with double gene and triple-gene constructs expressing SDF1. Jurkat cell migration using supernatant from the iPSC CMs shows that the transfection and SDF1 protein is functional and produces significant migration. Data shows a dose dependent migration of Jurkat cells, demonstrating successful expression and activity of functional polypeptides encoded by polynucleotides described herein, comprising linkers disclosed herein.

FIG. 12 shows cardiomyocytes from a dilated cardiomyopathy subject were transfected with double gene and triple-gene constructs encoding for SDF1. Data show SDF1 dependent Jurkat cell migration. SDF1 expressed by double gene and triple-gene vector constructs in Dilated Cardiomyopathy iPSC-CMs is functional.

FIG. 13 shows cardiomyocytes from a hypertrophic cardiomyopathy subject were transfected with double gene and triple-gene constructs encoding for SDF1. Data show SDF1 dependent Jurkat cell migration. SDF1 expressed by double gene and triple-gene constructs in hypertrophic cardiomyopathy iPSC-CMs is functional.

FIG. 14A and FIG. 14B shows a proliferation assay performed on HUVECS. FIG. 14A. shows that transfected cardiomyocytes from a DCM subject with double gene and triple-gene constructs produce functional VEGF protein able to induce HUVEC proliferation. (Note: Sample 8 was not tested in Run1, Run2.) FIG. 14B shows that transfected cardiomyocytes from a healthy subject with double gene and triple-gene constructs produce functional VEGF protein which is able to induce HUVEC proliferation.

FIG. 15A and FIG. 15B depict SDF1 protein expression in 293 T cells transfected with a double gene vector encoding for S100A1-SDF1. FIG. 15A. ELISA assay standard curve showing absorbance of SDF1. FIG. 15B. SDF1 concentration in the 293T supernatant. The data demonstrates successful expression of functional (i.e., biologically active) polypeptides encoded by polynucleotides described herein, comprising linkers disclosed herein.

FIG. 16A and FIG. 16B depict VEGF protein expression in 293 T cells transfected with a double gene vector encoding for S100A1-VEGF191. FIG. 16A. ELISA assay standard curve showing absorbance of VEGF. FIG. 16B. VEGF concentration in the 293T supernatant. This data demonstrates successful expression of functional (i.e., biologically active) polypeptides encoded by polynucleotides described herein, comprising linkers disclosed herein.

FIG. 17 A and FIG. 17B depict S100A1 protein expression in 293 T cells transfected with a double gene vector encoding for S100A1-SDF1. FIG. 17A. ELISA assay standard curve showing absorbance of S100A1. FIG. 17B. S100A1 concentration in the 293T lysate. This data demonstrates successful expression of functional (i.e., biologically active) polypeptides encoded by polynucleotides described herein, comprising linkers disclosed herein.

FIG. 18 shows a bar graph of S100A1 concentrations as produced by cardiomyocytes transfected with single-gene, double-gene and triple-gene constructs.

FIG. 19 shows a bar graph of SDF-1α concentrations as produced by cardiomyocytes transfected with single-gene, double-gene and triple-gene constructs.

FIG. 20 shows a bar graph of VEGF concentrations as produced by cardiomyocytes transfected with single-gene, double-gene and triple-gene constructs.

FIG. 21 shows Western blot (immunoblot) detection of S100A1 polypeptide cleavage as produced by cardiomyocytes transfected with single-gene, double-gene and triple-gene constructs.

FIG. 22 shows Western blot (immunoblot) detection of SDF-1α polypeptide cleavage as produced by cardiomyocytes transfected with single-gene, double-gene and triple-gene constructs.

FIG. 23 shows Western blot (immunoblot) detection of VEGF polypeptide cleavage as produced by cardiomyocytes transfected with single-gene, double-gene and triple-gene constructs.

FIG. 24 shows formation of dimeric S100A1 expressed from triple-gene constructs, using linkers of the invention, as detected via non-denaturing PAGE and immunoblotting.

FIG. 25A and FIG. 25B show a comparison of iPSC cells from a dilated cardiomyopathy patients vs. a healthy control in terms of FIG. 25A. beat rate of iPSC cells transfected with pStuffer, a vector encoding for pS100A1, or a vector encoding for S100A1 comprising a 2A tail. FIG. 25B. contractile duration of beating rate of iPSC cells transfected with pStuffer, a vector encoding for pS100A1, or a vector encoding for S100A1 comprising a 2A tail. The data indicates that the presence of a 2A tail at the 3′ end of a vector encoding for S100A1 does not affect the function of S100A1. (pStuffer is a plasmid with the same backbone configuration as pXoX, with the open reading frame (ORF) replaced with a non-expressing, similar-sized stuffer sequence.)

FIG. 26A, FIG. 26B, and FIG. 26C show a comparison of iPSC cells from a dilated cardiomyopathy patients vs. a healthy control in terms of FIG. 26A. beat rate of iPSC cells transfected with DNA1 (CAGStufferNXB-V2), DNA2 (CAG-S100A1 (no kozak), DNA3 (CAG-S100A1-fp2a-SDF1), DNA4 (CAG-S100A1-fp2a-SDF1-fp2a-VEGF), DNA 5(CAG-S100A1-CMV-SDF1-fp2a-VEGF191), or an untransfected control. FIG. 26B. contractile rate of iPSC cells transfected with DNA1, DNA2, DNA3, DNA4, DNA5, or an untransfected control. FIG. 26C. contractile duration of DNA1, DNA2, DNA3, DNA4, DNA5, or an untransfected control. Data shows the restoration of DCM contractile properties to healthy control levels.

FIG. 27A and FIG. 27 B show a triple-gene vector encoding for S100A1, SDF-1alpha, and VEGF165. FIG. 27A shows a schematic depiction of the pXoX triple gene vector encoding biologically active SDF1α, S100A1, and VEGF. FIG. 27B identifies individual gene elements in the triple-gene vector.

FIG. 28 shows data for expression of VEGF in a recombinase titration experiments. Data indicates integration of VEGF into genomic DNA.

FIG. 29 shows data for expression of SDF-1α in a recombinase titration experiments. Data indicates integration of SDF-1α into genomic DNA.

FIG. 30 shows data for expression of S100A1 in a recombinase titration experiments. Data indicates integration of S100A1 into genomic DNA.

FIGS. 31A-31C show in vivo therapeutic results via echocardiographic measurement of cardiac structure and as function of fractional shortening. FIG. 31A shows results as a function of percent fractional shortening. FIG. 31B shows left ventricle posterior wall thickness (cm) of the pStuffer construct as compared to the pXoX construct. FIG. 31C shows IVSd (cm) of the pStuffer construct as compared to pXoX construct. Values are presented as mean±SEM. **P<0.001 vs pStuffer.

FIG. 32 shows results of nuclear localization of PHH3 (phospho-histone H3 (Ser10)) Mitotic marker signal in cardiac cells demonstrating cardiomyocyte regeneration. Representative images of 4 out of 8 rats per plasmid. Scale bar is 25 μm.

FIG. 33 shows imaging results demonstrating negative results of ISL-1, a marker of early cardiac progenitor cells confirming progenitor cells are not involved in regeneration. Observed cardiomyocyte regeneration is, therefore, a result of UTMD-pXoX gene therapy. Representative images of 4 out of 8 rats per plasmid. Scale bar is 25 μm.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

The S100 family of proteins includes, for instance, S100A1, S100A2, S100A3, S100A4, S100A5, S100A6, S100A7, S100A8, S100A9, S100A10, S100A11, S100A12, S100A13, S100A14, S100A15, and S100A16. In some cases are proteins similar to a S100A protein for instance S100A7L (S100 calcium binding protein A7-like). Other S100 proteins include S100B, S100G, S100P and S100Z and variants and fragments thereof which may be included in embodiments described herein. Also included in embodiments described herein are 5100 proteins which are the result of pseudogene products that may be expressed by vectors described herein.[61]

The SDF family of proteins include for instance, a protein encoded by an antimicrobial gene that can encode a stromal cell-derived alpha chemokine member of an intercrine family.[62] Embodiments described herein can comprise encoded proteins which may function as ligands for a G-protein coupled receptor, chemokine (C-X-C motif) receptor 4, and may play a role in many diverse cellular functions, including embryogenesis, immune surveillance, inflammation response, tissue homeostasis, and tumor growth and metastasis. Also included are transcript variants encoding different isoforms, and said isoforms and transcript variants may be expressed by vectors described herein. In some embodiments described herein, are SDF family proteins wherein the first two cysteine residues are separated by one amino acid (C-X-C chemokine). The following protein isoforms have been identified in humans and may be expressed by vectors described herein and included in methods and compositions described herein: SDF-1 Alpha, SDF-1 Beta, SDF-1 Gamma, SDF-1 Delta, SDF-1 Epsilon and SDF-1 Theta, and fragments and variants thereof.

The VEGF family of proteins described herein includes members of the PDGF/VEGF growth factor family. For instance included in methods and compositions described herein, can be splice variants and isoforms and fragments and derivatives of VEGF121, VEGF121b, VEGF145, VEGF165, VEGF165b, VEGF189, VEGF191, VEGF206. [63].

In some cases, the S100 polypeptide can be a S100A1, and any functional derivative thereof. In some embodiments, the second functional polypeptide can be an angiogenic polypeptide. An angiogenic polypeptide can be a vascular endothelial growth factor (VEGF) polypeptide. In a few embodiments, a VEGF polypeptide is selected from a group consisting of VEGF121, VEGF121b, VEGF145, VEGF165, VEGF165b, VEGF189, VEGF191, VEGF206, fragments and variants thereof. In certain embodiments, VEGF 165 has a sequence comprising amino acids 27-191 of a VEGF191 sequence disclosed herein. In certain embodiments, VEGF 165 has a sequence comprising amino acids 27, 28, 29, 30, 31, 32, 33, or 34 to 180, 181, 182, 183, 184, 185, 186, 187, 188, 189, 190, or 191 of a VEGF191 sequence disclosed herein. In certain embodiments, VEGF 165 has a sequence comprising amino acids 30-185 of a VEGF191 sequence disclosed herein. In some cases, a VEGF polypeptide described herein can comprise a sequence with at least 45%, 50%, 55%, 60%, 65%, 70%, 75%, 80%, 85%, 90%, 95%, or 99% identity to the sequence of VEGF 191 or VEGF165 disclosed herein. In some cases, a VEGF polypeptide can comprise a sequence with at least 45%, 50%, 55%, 60%, 65%, 70%, 75%, 80%, 85%, 90%, 95%, or 99% identity to the sequence of VEGF191 polypeptide disclosed herein.

In other embodiments, the third functional polypeptide can be a chemokine. The chemokine can be a stromal cell derived factor 1 (SDF) polypeptide. An SDF polypeptide can be selected from the group consisting of SDF1, SDF-1α, SDF-1p, fragments and variants thereof.[62]. An SDF polypeptide can comprise a sequence with at least 45%, 50%, 55%, 60%, 65%, 70%, 75%, 80%, 85%, 90%, 95%, or 99% identity to the sequence of SDF1 polypeptide disclosed herein.

In some embodiments described herein, an S100 polypeptide can be connected to the second polypeptide by a first polypeptide linker. In some cases, the polypeptide linker can be a cleavable linker or an uncleavable linker.

Provided herein are embodiments wherein a polypeptide construct encoded by a polynucleotide described herein can comprise a calcium-binding protein (S100) polypeptide, a second functional polypeptide, and further comprise a third functional polypeptide. In some cases, the third functional polypeptide can be any one of a cytokine, a chemokine, an angiogenic polypeptide, and any functional derivative thereof.

In certain embodiments, the third functional polypeptide can be an angiogenic polypeptide. In some embodiments, the angiogenic polypeptide can be a VEGF polypeptide. A VEGF polypeptide can be selected from a group consisting of VEGF121, VEGF121b, VEGF145, VEGF165, VEGF165b, VEGF189, VEGF191, VEGF206, fragments and variants thereof. A VEGF polypeptide can comprise a sequence with at least 45%, 50%, 55%, 60%, 65%, 70%, 75%, 80%, 85%, 90%, 95%, or 99% identity to the sequence of the VEGF165 polypeptide. In some cases, a VEGF polypeptide can comprise a sequence with at least 45%, 50%, 55%, 60%, 65%, 70%, 75%, 80%, 85%, 90%, 95%, or 99% identity to the sequence of VEGF191 polypeptide.

In some embodiments, the third functional polypeptide can be a chemokine. In certain embodiments, the chemokine can be a SDF polypeptide. In some embodiments, the SDF polypeptide is selected from a group consisting of SDF1, SDF-1α, SDF-1P, fragments and variants thereof. In some cases, the SDF polypeptide can comprise a sequence with at least 45%, 50%, 55%, 60%, 65%, 70%, 75%, 80%, 85%, 90%, 95%, or 99% identity to the sequence of SDF1 polypeptide described herein.

In some embodiments, the third functional polypeptide can be connected to at least one of said S100 polypeptide and said second functional polypeptide by a second polypeptide linker which can be optionally cleavable. Polypeptide linkers can be independently selected from the linkers described herein.

Provided herein are polynucleotides encoding at least one polypeptide construct comprising at least a first polypeptide and a second polypeptide, wherein a first polypeptide can be an angiogenic polypeptide and a second polypeptide can be a chemokine or variant or fragment thereof.

In some cases, an angiogenic polypeptide can be a VEGF polypeptide selected from a group consisting of VEGF121, VEGF121b, VEGF145, VEGF165, VEGF165b, VEGF189, VEGF191, VEGF206, fragments and variants thereof. A polypeptide can comprise a sequence with at least 45%, 50%, 55%, 60%, 65%, 70%, 75%, 80%, 85%, 90%, 95%, or 99% identity to the sequence of a VEGF polypeptide.

Provided are VEGF polypeptides comprising a sequence with at least 45%, 50%, 55%, 60%, 65%, 70%, 75%, 80%, 85%, 90%, 95%, or 99% identity to the sequence of VEGF191. In some cases, a second polypeptide can be an SDF polypeptide. An SDF polypeptide can be selected from the group consisting of SDF1, SDF-1α, SDF-1p, fragments and variants thereof. In some cases, an SDF polypeptide can comprise a sequence with at least 45%, 50%, 55%, 60%, 65%, 70%, 75%, 80%, 85%, 90%, 95%, or 99% identity to the sequence of SDF1.

Provided herein are methods of improving vasculogenesis in a subject comprising providing to said subject a therapeutically effective amount of a composition comprising a polynucleotide described herein or a polypeptide encoded by a polynucleotide described herein.

Provided herein are expression vectors, comprising at least one promoter operably linked to at least two polypeptides selected from a SDF1 polypeptide, a S100A1 polypeptide, a VEGF polypeptide and fragments and variants thereof. In some cases, at least two polypeptides are connected by a linker. In some cases, at least one promoter can be selected from the group consisting of CAG promoter, CMV promoter, SV40 promoter, adenovirus promoter, Beta actin promoter, metallothionin promoter, EF1a promoter, myosin light chain promoter, myosin heavy chain promoter, NCX1 promoter and other suitable cardiac promoters. An expression vector can be a cardiac expression vector.

Provided herein are polypeptide constructs comprising at least a first polypeptide, and a second polypeptide, wherein the first polypeptide can be a calcium binding protein, or variant or fragment thereof and the second polypeptide can be an angiogenic polypeptide, a chemokine, or variant or fragment thereof. Also disclosed are polypeptide constructs comprising at least a first polypeptide, a second polypeptide, and a third polypeptide wherein a first polypeptide can be an angiogenic polypeptide or variant or fragment thereof, a second polypeptide can be a chemokine, or variant or fragment thereof; and a third polypeptide can be a calcium binding protein, or variant or fragment thereof. In some cases, a polypeptide construct described herein can comprise S100A1, a variant or a fragment thereof. In some cases, a polypeptide construct can comprise SDF1, a variant or a fragment thereof. In further cases, a polypeptide construct described herein can comprise VEGF, a variant or a fragment thereof.

Provided are pharmaceutical compositions comprising a polynucleotide described herein, or a polypeptide construct provided herein, or a polypeptide construct encoded by a polynucleotide described herein, and a pharmaceutically acceptable excipient. Provided herein are methods of treating a cardiac diseases and disorders in a subject comprising providing to the subject a therapeutically effective amount of a composition comprising a polynucleotide described herein, or a polypeptide construct provided herein, or a polypeptide construct encoded by a polynucleotide described herein.

In some embodiments, a polynucleotide construct encoding a polypeptide construct described herein further comprises a third functional polypeptide connected by a second linker polypeptide to the second functional polypeptide. In some cases, the third functional polypeptide can be selected from a list consisting of VEGF, SDF1, S100A1, variants and derivatives thereof. In some cases, the second linker can be the same or different from the first linker described herein. In some cases, the linkers and functional polypeptides can be expressed in-frame.

Provided herein are cells comprising a polynucleotide described herein.

Provided herein is a method comprising: contacting at least one cardiac cell with a polynucleotide encoding a polypeptide construct comprising at least a first polypeptide and a second polypeptide, wherein a first polypeptide is an angiogenic polypeptide variant or fragment thereof; and a second polypeptide is at least one of a chemokine, a calcium binding protein, or variant or fragment thereof. In some embodiments, an angiogenic polypeptide variant or fragment thereof can be a VEGF polypeptide selected from a group consisting of VEGF121, VEGF121b, VEGF145, VEGF165, VEGF165b, VEGF189, VEGF191, VEGF206, fragments and variants thereof. A VEGF polypeptide can comprise a sequence with at least 45%, 50%, 55%, 60%, 65%, 70%, 75%, 80%, 85%, 90%, 95%, or 99% identity to the sequence of VEGF165. In certain embodiments, VEGF 165 has a sequence comprising amino acids 27-191 of a VEGF191 sequence disclosed herein. In certain embodiments, VEGF 165 has a sequence comprising amino acids 27, 28, 29, 30, 31, 32, 33, or 34 to 180, 181, 182, 183, 184, 185, 186, 187, 188, 189, 190, or 191 of a VEGF191 sequence disclosed herein. In certain embodiments, VEGF 165 has a sequence comprising amino acids 30-185 of a VEGF191 sequence disclosed herein. In some cases, a VEGF polypeptide described herein can comprise a sequence with at least 45%, 50%, 55%, 60%, 65%, 70%, 75%, 80%, 85%, 90%, 95%, or 99% identity to the sequence of VEGF 191 or VEGF165 disclosed herein. A VEGF polypeptide can comprise a sequence with at least 45%, 50%, 55%, 60%, 65%, 70%, 75%, 80%, 85%, 90%, 95%, or 99% identity to the sequence of VEGF191. In some cases, a second polypeptide can be a chemokine. A chemokine can be a stromal cell-derived factor 1 (SDF) polypeptide. In some cases, an SDF polypeptide useful in compositions and methods provided herein can be selected from the group consisting of SDF1, SDF-1α, SDF-1p, fragments and variants thereof. An SDF polypeptide can comprise a sequence with at least 45%, 50%, 55%, 60%, 65%, 70%, 75%, 80%, 85%, 90%, 95%, or 99% identity to SDF1-α.

In some cases, a polynucleotide encoding a polypeptide construct can be introduced ex vivo. In certain embodiments, a polynucleotide encoding a polypeptide construct can be introduced in vivo. An introduction in vivo can be selected from a group consisting of percutaneous coronary artery catheterization, coronary venous blockade, cardiac recirculation, antegrade coronary artery infusion, retrograde perfusion, direct injection, and any combination thereof.

Provided herein are methods of treating a cardiac disease or disorder in a subject (for example, but not limited to, by improving vasculogenesis, cardiac function or cardiac remodeling) in a subject comprising administering to said subject an amount of a polynucleotide encoding a construct comprising a VEGF polypeptide, and at least one of a SDF polypeptide and a S100 polypeptide. A VEGF polypeptide in an embodiment described herein can be selected from a group consisting of VEGF121, VEGF121b, VEGF145, VEGF165, VEGF165b, VEGF189, VEGF191, VEGF206, fragments and variants thereof. A VEGF polypeptide can comprise a sequence with at least 45%, 50%, 55%, 60%, 65%, 70%, 75%, 80%, 85%, 90%, 95%, or 99% identity to the sequence of VEGF165. In certain embodiments, VEGF 165 has a sequence comprising amino acids 27-191 of a VEGF191 sequence disclosed herein. A VEGF polypeptide can comprise a sequence with at least 45%, 50%, 55%, 60%, 65%, 70%, 75%, 80%, 85%, 90%, 95%, or 99% identity to the sequence of VEGF191. An SDF polypeptide can be selected from the group consisting of SDF1, SDF-1α, SDF-13, fragments and variants thereof. An S100 polypeptide can be S100A1, fragment or variant thereof.

In certain embodiments, a subject treated with methods and compositions of the invention can have a congestive heart failure (CHF). A subject can have a left ventricular assist device (LVAD) in place. A subject can have a cardiomyopathy. A cardiomyopathy can be selected from the group consisting of dilated cardiomyopathy (DCM), hypertrophic cardiomyopathy (HCM), restrictive cardiomyopathy (RCM), arrhythmogenic right ventricular cardiomyopathy (ARVC), and any combination thereof.

In further embodiments, a subject treated with methods and compositions of the invention can have any one or more of: aneurysm, atherosclerosis, congenital heart defect, pericardial disorder, acute decompensated heart failure, angina, arteriosclerotic heart disease, athletic heart syndrome, atrioventricular fistula, autoimmune heart disease, brown atrophy of the heart, cardiac amyloidosis, cardiac arrhythmia, cardiac asthma, cardiac contractility modulation, cardiac syndrome x, cardiogenic shock, cardiomegaly, cardiomyopathy, cardiophobia, cardiorenal syndrome, cardiotoxicity, cardiovascular disease, carditis, chronic rheumatic heart diseases, coeur en sabot, coronary artery aneurysm, coronary artery anomaly, coronary artery disease, coronary artery dissection, coronary artery ectasia, coronary occlusion, coronary steal, coronary thrombosis, coronary vasospasm, coxsackievirus-induced cardiomyopathy, diastolic heart failure, dressler syndrome, duroziez's disease, eisenmenger's syndrome, embryocardia, embryonic recall, endocardial fibroelastosis, heart failure with preserved ejection fraction, heart neoplasia, high-output heart failure, hyperdynamic precordium, hypertensive heart disease, idiopathic giant-cell myocarditis, inflammatory heart disease, interventricular dyssynchrony, intraventricular dyssynchrony, ischemic heart disease, isolated atrial amyloidosis, keshan disease, kounis syndrome, mydicar, myocardial bridge, myocardial disarray, myocardial rupture, myocardial scarring, myocardial stunning, myocarditis, nonbacterial thrombotic endocarditis, ostial disease, peripheral arterial disease, phosphorus and non-atherosclerotic heart disease, postpericardiotomy syndrome, pressure-controlled intermittent coronary sinus occlusion (picso), recovery from cardiopulmonary resuscitation, recovery from traumatic cardiac arrest, right axis deviation, rheumatic heart disease, roemheld syndrome, saturated fat and cardiovascular disease controversy, scar-fc, shone's syndrome, subacute bacterial endocarditis, valvular heart disease, ventricular aneurysm, and viral cardiomyopathy.

Provided herein are methods of treating or preventing a cardiovascular condition in a subject comprising: administering to a subject an amount of a construct comprising a S100A polypeptide, and a second functional polypeptide, wherein said second functional polypeptide is at least one of a cytokine, a chemokine and an angiogenic polypeptide. In some cases, an administration to a subject can be performed preventively. In some cases, an administration to a subject can be performed therapeutically. In some cases, a subject can be administered at least one additional treatment to said subject. In some cases, a second functional polypeptide can be VEGF, SDF, or a combination thereof.

Provided herein are methods comprising administering to a subject at least one non-viral vector comprising a polynucleotide encoding a polypeptide sequence described herein comprising at least two functional proteins or portions thereof; at least one promotor; and at least one engineered recombination site; wherein said at least one promoter drives expression of said at least two functional proteins. In some cases, at least one promotor can be constitutive. In some cases, at least one promoter can be tissue-specific. In some cases, at least one promoter can be inducible. In some cases, at least one tissue-specific promotor is a myosin light chain (MLC) promoter. In some cases, an inducible promoter is a small molecule ligand-inducible two polypeptide ecdysone receptor-based gene switch.

An inducible promoter utilizes a ligand for dose-regulated control of expression of said at least two genes. In some cases, a ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof. A linker can be a cleavable linker.

In some embodiments, a first functional polypeptide and a second functional polypeptide connected by a first linker polypeptide, wherein the first linker polypeptide comprises a sequence with at least 40%, 50%, 60%, 80% or 100% identity to the sequence APVKQ (SEQ ID NO: 42). In some cases, the linker polypeptide comprises a sequence selected from the group consisting of APVKQ (SEQ ID NO: 42), GPVKQ (SEQ ID NO: 43), VPVKQ (SEQ ID NO: 44), IPVKQ (SEQ ID NO: 45), MPVKQ (SEQ ID NO: 46), APIKQ (SEQ ID NO: 47), GPIKQ (SEQ ID NO: 48), VPIKQ (SEQ ID NO: 49), IPIKQ (SEQ ID NO: 50), MPIKQ (SEQ ID NO: 51), APAKQ (SEQ ID NO: 52), GPAKQ (SEQ ID NO: 53), VPAKQ (SEQ ID NO: 54), IPAKQ (SEQ ID NO: 55), MPAKQ (SEQ ID NO: 56), APVRQ (SEQ ID NO: 57), GPVRQ (SEQ ID NO: 58), VPVRQ (SEQ ID NO: 59), IPVRQ (SEQ ID NO: 60), MPVRQ (SEQ ID NO: 61), APIRQ (SEQ ID NO: 62), GPIRQ (SEQ ID NO: 63), VPIRQ (SEQ ID NO: 64), IPIRQ (SEQ ID NO: 65), MPIRQ (SEQ ID NO: 66), APARQ (SEQ ID NO: 67), GPARQ (SEQ ID NO: 68), VPARQ (SEQ ID NO: 69), IPARQ (SEQ ID NO: 70), MPARQ (SEQ ID NO: 71), APVKN (SEQ ID NO: 72), GPVKN (SEQ ID NO: 73), VPVKN (SEQ ID NO: 74), IPVKN (SEQ ID NO: 75), MPVKN (SEQ ID NO: 76), APIKN (SEQ ID NO: 77), GPIKN (SEQ ID NO: 78), VPIKN (SEQ ID NO: 79), IPIKN (SEQ ID NO: 80), MPIKN (SEQ ID NO: 81), APAKN (SEQ ID NO: 82), GPAKN (SEQ ID NO: 83), VPAKN (SEQ ID NO: 84), IPAKN (SEQ ID NO: 85), MPAKN (SEQ ID NO: 86), APVRN (SEQ ID NO: 87), GPVRN (SEQ ID NO: 88), VPVRN (SEQ ID NO: 89), IPVRN (SEQ ID NO: 90), MPVRN (SEQ ID NO: 91), APIRN (SEQ ID NO: 92), GPIRN (SEQ ID NO: 93), VPIRN (SEQ ID NO: 94), IPIRN (SEQ ID NO: 95), MPIRN (SEQ ID NO: 96), APARN (SEQ ID NO: 97), GPARN (SEQ ID NO: 98), VPARN (SEQ ID NO: 99), IPARN (SEQ ID NO: 100) and MPARN (SEQ ID NO: 101). In some cases, a polypeptide linker may also include one or more GS linker sequences, for instance (GS)n (SEQ ID NO: 109), (SG)n (SEQ ID NO: 110), (GSG)n (SEQ ID NO: 111) and (SGS)n (SEQ ID NO: 112) wherein n can be any number from zero to fifteen. In some embodiments, the first functional polypeptide can be different from the second functional polypeptide.

In some embodiments, a method can further comprise administering to a subject at least one secondary vector. At least one secondary vector can be an mRNA. At least one secondary vector can be a plasmid. In some embodiments, at least one secondary vector can comprise at least one recombinase. A recombinase can be selected from the group consisting of a Listeria monocytogenes phage recombinase, a Streptococcus pyogenes phage recombinase, a Bacillus subtilis phage recombinase, a Mycobacterium tuberculosis phage recombinase and a Mycobacterium smegmatis phage recombinase. In some embodiments, at least two genes can be integrated into a genome of a subject by a recombinase. In some embodiments, at least one recombination site can be a phage genomic recombination attachment site (attP) or a bacterial genomic recombination attachment site (attB). In some embodiments, a recombinase used as described in the invention herein comprises a site-specific serine recombinase; such as, but not limited to, SpBC2 recombinase (see, for example, U.S. Pat. No. 9,034,650 issued May 19, 2015 (U.S. Pub. No. 2006/0172377) which is hereby incorporated by reference herein in its entirety).

A vector can be good manufacturing practices (GMP) compatible.

Provided are polypeptides comprising a sequence with at least 40%, 50% 60%, 80%, or 100% identity to a sequence selected from the group consisting of SEQ ID NO: 32, SEQ ID NO: 34, SEQ ID NO: 36, SEQ ID NO: 38, and SEQ ID NO: 40. In some embodiments, the polypeptide comprises a sequence with at least 40%, 50%, 60%, 70%, 75%, 80%, 85%, 90%, 95%, or 99% identity to a sequence selected from the group consisting of SEQ ID NO: 32, SEQ ID NO: 34, SEQ ID NO: 36, SEQ ID NO: 38, and SEQ ID NO: 40.

Provided are polypeptides comprising a sequence with at least 40%, 50%, 60%, 80%, or 100% identity to a sequence selected from the group consisting of SEQ ID NO: 102, SEQ ID NO: 104 and SEQ ID NO: 106. In some embodiments is a polypeptide comprising a sequence with at least 45%, 50%, 55%, 60%, 65%, 70%, 75%, 80%, 85%, 90%, 95%, or 99% identity to a sequence selected from the group consisting of SEQ ID NO: 102, SEQ ID NO: 104 and SEQ ID NO: 106.

Provided is a polypeptide comprising a sequence with at least 45%, 50%, 55%, 60%, 65%, 70%, 75%, 80%, 85%, 90%, 95%, or 99% identity to a polypeptide sequence encoded by the nucleic acid sequence of SEQ ID NO. 108.

Provided herein are polynucleotides encoding a polypeptide construct comprising a first functional polypeptide and a second functional polypeptide connected by a first linker polypeptide, wherein the first linker polypeptide comprises a sequence with at least 40%, 50%, 60%, 80%, or 100% identity to the sequence APVKQ. In some cases, the linker polypeptide comprises a sequence selected from the group consisting of APVKQ (SEQ ID NO: 42). In some cases, the linker polypeptide comprises a sequence selected from the group consisting of APVKQ (SEQ ID NO: 42), GPVKQ (SEQ ID NO: 43), VPVKQ (SEQ ID NO: 44), IPVKQ (SEQ ID NO: 45), MPVKQ (SEQ ID NO: 46), APIKQ (SEQ ID NO: 47), GPIKQ (SEQ ID NO: 48), VPIKQ (SEQ ID NO: 49), IPIKQ (SEQ ID NO: 50), MPIKQ (SEQ ID NO: 51), APAKQ (SEQ ID NO: 52), GPAKQ (SEQ ID NO: 53), VPAKQ (SEQ ID NO: 54), IPAKQ (SEQ ID NO: 55), MPAKQ (SEQ ID NO: 56), APVRQ (SEQ ID NO: 57), GPVRQ (SEQ ID NO: 58), VPVRQ (SEQ ID NO: 59), IPVRQ (SEQ ID NO: 60), MPVRQ (SEQ ID NO: 61), APIRQ (SEQ ID NO: 62), GPIRQ (SEQ ID NO: 63), VPIRQ (SEQ ID NO: 64), IPIRQ (SEQ ID NO: 65), MPIRQ (SEQ ID NO: 66), APARQ (SEQ ID NO: 67), GPARQ (SEQ ID NO: 68), VPARQ (SEQ ID NO: 69), IPARQ (SEQ ID NO: 70), MPARQ (SEQ ID NO: 71), APVKN (SEQ ID NO: 72), GPVKN (SEQ ID NO: 73), VPVKN (SEQ ID NO: 74), IPVKN (SEQ ID NO: 75), MPVKN (SEQ ID NO: 76), APIKN (SEQ ID NO: 77), GPIKN (SEQ ID NO: 78), VPIKN (SEQ ID NO: 79), IPIKN (SEQ ID NO: 80), MPIKN (SEQ ID NO: 81), APAKN (SEQ ID NO: 82), GPAKN (SEQ ID NO: 83), VPAKN (SEQ ID NO: 84), IPAKN (SEQ ID NO: 85), MPAKN (SEQ ID NO: 86), APVRN (SEQ ID NO: 87), GPVRN (SEQ ID NO: 88), VPVRN (SEQ ID NO: 89), IPVRN (SEQ ID NO: 90), MPVRN (SEQ ID NO: 91), APIRN (SEQ ID NO: 92), GPIRN (SEQ ID NO: 93), VPIRN (SEQ ID NO: 94), IPIRN (SEQ ID NO: 95), MPIRN (SEQ ID NO: 96), APARN (SEQ ID NO: 97), GPARN (SEQ ID NO: 98), VPARN (SEQ ID NO: 99), IPARN (SEQ ID NO: 100) and MPARN (SEQ ID NO: 101). In some cases, a polypeptide linker may also include one or more GS linker sequences, for instance (GS)n (SEQ ID NO: 113), (SG)n (SEQ ID NO: 114), (GSG)n (SEQ ID NO: 115) and (SGS)n (SEQ ID NO: 116) wherein n can be any number from one to fifteen. In some embodiments, the first functional polypeptide can be different from the second functional polypeptide. In other cases, at least one of said first and second functional polypeptides can be a protein, hormone, antibody, glycoprotein or derivative or fragment thereof.

Provided herein are polynucleotides encoding a polypeptide construct comprising a furin polypeptide and a 2A polypeptide, wherein the furin polypeptide and the 2A polypeptide are connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), proline (Pro)(P), phenylalanine (Phe)(F), methionine (Met)(M), tryptophan (Trp)(W).

Provided are polypeptides encoded by polynucleotides described herein, and compositions comprising these polypeptides. Also provided are therapeutic, and/or diagnostic methods comprising contacting an individual with a polynucleotide disclosed herein or a polypeptide expressed therefrom or derivative, or conjugate thereof.

Provided are methods of obtaining an improved expression of a polypeptide construct comprising: providing a polynucleotide encoding said polypeptide construct comprising a first functional polypeptide and a second functional polypeptide, wherein said first functional polypeptide and second functional polypeptide are connected by a linker polypeptide comprising a sequence with at least 60% identity to the sequence APVKQ (SEQ ID NO: 42); and expressing said polynucleotide in a host cell, wherein said expressing results in an improved expression of the polypeptide construct as compared to a corresponding polypeptide construct that does not have a linker polypeptide comprising a sequence with at least 60% identity to the sequence APVKQ (SEQ ID NO: 42).

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The term “cardiovascular disease” and “circulatory disorder” are used interchangeably, and are used herein to describe a disease or disorder which is caused by damage to the circulatory system and which damage can be reduced and/or alleviated by providing a therapeutically effective amount of a polynucleotide described herein or a polypeptide construct encoded by a polynucleotide disclosed herein, to damaged areas of the heart and/or circulatory system of the subject. As used herein, the term “circulatory damage” is used to refer to injury to the circulatory system that may be caused be any of a number of diseases or disorders. Exemplary cardiovascular diseases which may be treated using a polynucleotide described herein or a polypeptide construct encoded by a polynucleotide disclosed herein and methods according to the present invention include for example, myocardial infarct, cardiomyopathy, peripheral vascular disease, congenital heart disease, other genetic diseases, and injury or trauma caused by ischemia, accidents, environmental insult. In addition, a therapeutically effective amount polynucleotide described herein or a polypeptide construct encoded by a polynucleotide disclosed herein may be used to reduce and/or eliminate the effects on the central nervous system of a heart attack in a subject, which is otherwise caused by lack of blood flow or ischemia to a site in the brain of said subject or which has occurred from physical injury to the brain and/or spinal cord.

The term “vector” or grammatical equivalents as used herein can refer any polynucleotide construct capable of directing the expression of a polypeptide construct of interest and which is useful in transferring the polypeptide construct of interest into target cells. Thus, the term includes cloning and expression vehicles, as well as integrating vectors.

Based on an increasing number of patients with congestive heart failure (CHF) with limited options (for example, commonly resulting in the requirement for destination left ventricular assist devices (LVAD) therapy), biologic options to promote recovery from CHF are needed. Thus, one embodiment of the present invention provides a triple effector plasmid-based DNA—pXoX (SDF1α, S100A1, and VEGF) to improve cardiac performance (for, example, but not limited to, as assessed by measuring the number and duration of temporary weans from LVAD support in patients who have been implanted with destination LVADS at least thirty days post-surgery). Implantation of LVADs alone can result in improved cardiac function, most of which occurs within the first 30 days or so following implantation. Also, inflammatory processes which are normally associated with surgery recover to a new baseline by 30 days post-surgery, which may result in decreased risk of treatment with pXoX to this high risk patient population.

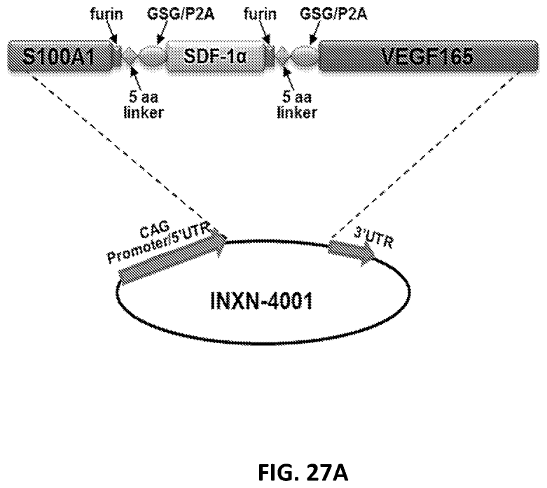

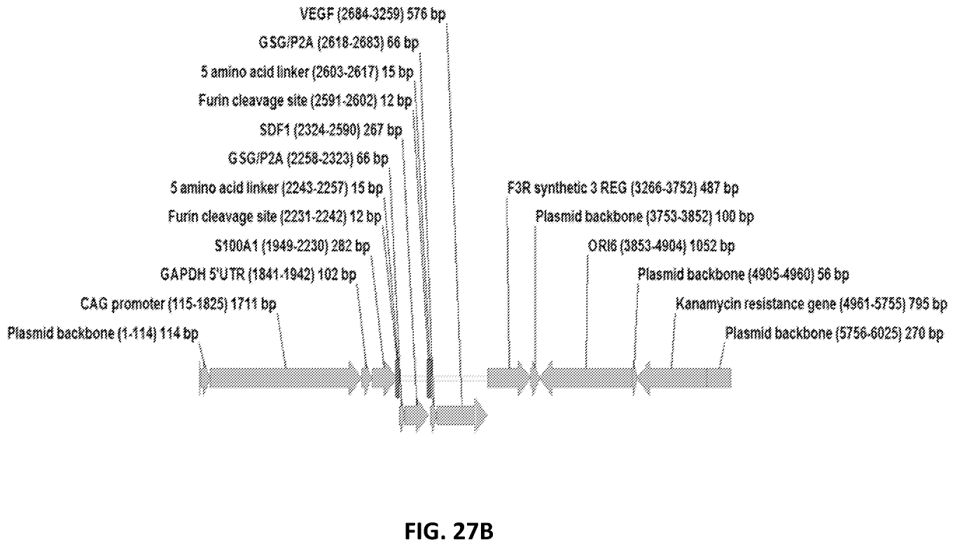

The pXoX plasmid constitutively expresses three proteins (S100A1, SDF-1α, and VEGF165) under the control of a CAG promoter, as a single mRNA that is processed into the individual effector proteins during translation via cleavage by the combined activity of furin and 2A self-cleaving peptides (fp2a). The plasmid backbone contains a kanamycin selection marker and an origin of replication derived from pBR322. An illustration of the pXoX plasmid is provided below in FIG. 27A and FIG. 27B.

The present invention also provides linkers for use, for instance, in compositions and methods useful for treatment of cardiac pathologies, conditions, and disorders (e.g., heart failure, cardiomyopathy, arrhythmia, pericardial disease, aorta disease, marfan syndrome and coronary artery disease.) using novel multigenic vectors, combinations of genetic sequences, proteins (polypeptides), and techniques described and provided for herein. As used herein, the term “linker” can mean any polynucleotide or polypeptide sequence that is used to connect any one polynucleotide or polypeptide sequence with another polynucleotide or polypeptide sequence, respectively (e.g., First GOI-Linker-Second GOI-Linker-Third GOI-Linker-etc. . . . ). For example, in some instances a linker may be a polynucleotide open-reading frame encoding a fusion protein such that a first polypeptide sequence is covalently linked (“fused”) by an intervening amino acid sequence to a second polypeptide sequence (and so on for third, fourth, fifth, etc. . . . subsequent polypeptides in the same open-reading frame). As used herein a “linker” may also be a non-coding, linking polynucleotide sequence such as a promoter (e.g., CAG promoter or CMV promoter) or an IRES (Internal Ribosome Entry Site) which function to couple a first polynucleotide coding region (ORF) to a second polynucleotide coding region (ORF) (and so on for third, fourth, fifth, etc. . . . subsequent polynucleotide open-reading frames).

Gene therapy as used herein refers to the transfer of polynucleotide described herein (e.g., DNA or RNA) of interest into a subject to treat or prevent a genetic or acquired disease or condition. Polynucleotide of interest can encode a product (e.g., a polypeptide construct) whose in vivo production is desired. In some embodiments, the polynucleotide of interest also encodes a suicide gene. In some cases, gene therapy can be used to treat a cardiovascular condition described herein.

### Vectors

Provided herein can be vectors for gene therapy of a disorder comprising using a polynucleotide described herein. A vector can be utilized for introducing exogenous nucleic acids such as DNA, mRNA, small interfering RNA (siRNA), microRNA (miRNA) or antisense oligonucleotides. Given the large size and the negative charge of these macromolecules, their delivery can be mediated by carriers or vectors. Vectors of interest include, in particular, any episomal vector, e.g. viral vectors, plasmid vectors, artificial chromosomes, mini-circles and the like.

Gene delivery systems can be classified into two categories, non-viral systems and recombinant viral systems, each of which can have unique profiles in gene transfer expression. Non-viral vectors can include naked plasmid DNA, liposomal DNA complexes, polymer-carried DNA, and oligonucleotides. Plasmids can be double-stranded circular DNA-containing transgenes encoding proteins of interest, and also have enhancer and promoter sequences. A vector can be a non-viral vector.

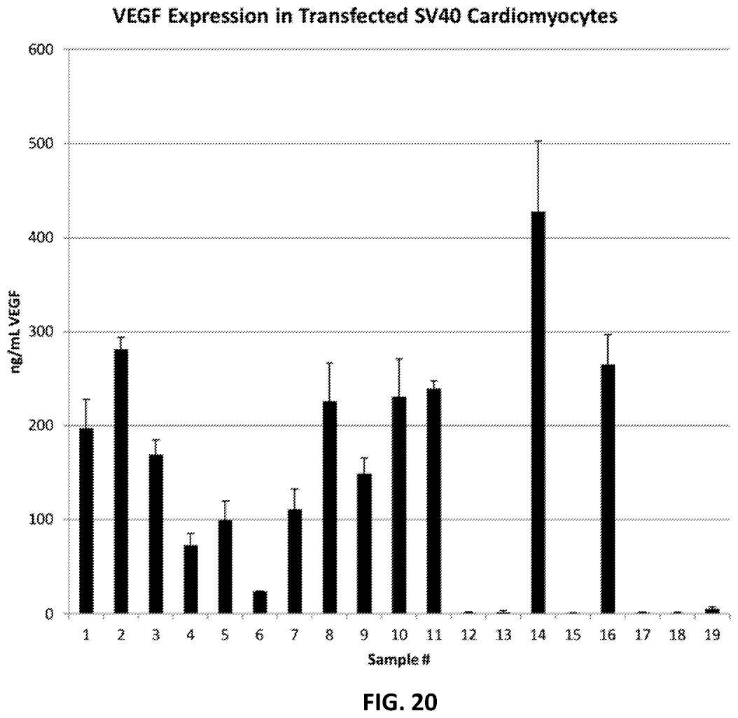

A vector can comprise genes or fragments or variants thereof, wherein at least one gene, fragment or variant thereof is connected to another gene, fragment or variant thereof by a linker disclosed herein. A vector can comprise a single-gene or multiple genes, or fragments or variants thereof. A vector can comprise genes, or fragments or variants thereof, wherein at least one gene, fragment or variant thereof can be connected to another gene, fragment or variant thereof by a linker disclosed herein. For example, a vector can be singe gene, double gene (see, e.g. Table 1), or triple-gene, wherein at least one gene, fragment or variant thereof is connected to another gene, fragment or variant thereof by a linker disclosed herein. A vector can comprise a number of genes from 1 to 10, or fragments or variants thereof. A vector can comprise 1, 2, 3, 4, 5, 6, 7, 8, 9, or 10 genes, fragments or variants thereof. An exemplary triple-gene vector is shown in FIG. 20 A and FIG. 20 B An exemplary vector sequence is shown in Table 5. In some cases, a vector can allow the simultaneous expression of two, three, or more polypeptides separately but from the same RNA transcript. A vector can also comprise a reporter polypeptide. For example, a vector can express one, two, three or more polypeptides and a reporter polypeptide.

In some cases, a vector can be constructed with at least one gene or fragment thereof. In other cases, a vector may contain up to 10 genes or fragments thereof. Genes or fragments thereof that can be expressed may be described by SEQ ID No. 102 to 107. Any homologs, isoforms, precursors, or modified portions thereof of SEQ ID No. 102 to 107 may also be expressed by vectors described herein. Genes or any fragment thereof can be separated by a linker described herein. Various linkers that may be utilized in a vector can comprise sequences shown in for instance SEQ ID No.32 to SEQ ID No. 41. In other cases, relevant linkers that can be utilized in a vector can comprise for instance a sequence shown in any of SEQ ID No. 42 to 101. For example, a portion of a gene can be at least one of a SDF gene (encoded by SEQ ID NO: 103 or 60%, 70%, 80%, 90%, or 95% identity thereto), a VEGF gene (encoded by SEQ ID NO: 107 or 60%, 70%, 80%, 90%, or 95% identity thereto) and a S100A gene (encoded by SEQ ID NO: 105 or 60%, 70%, 80%, 90%, or 95% identity thereto), which can be followed by a first linker sequence, a second gene or portion thereof which is selected from a SDF gene (encoded by SEQ ID NO: 103 or 60%, 70%, 80%, 90%, or 95% identity thereto), a VEGF gene (encoded by SEQ ID NO: 107 or 60%, 70%, 80%, 90%, or 95% identity thereto) and a S100A gene (encoded by SEQ ID NO: 105 or 60%, 70%, 80%, 90%, or 95% identity thereto), optionally followed by a second linker sequence, and subsequently a third gene or portion thereof selected from a SDF gene (encoded by SEQ ID NO: 103 or 60%, 70%, 80%, 90%, or 95% identity thereto), a VEGF gene (encoded by SEQ ID NO: 107 or 60%, 70%, 80%, 90%, or 95% identity thereto) and a S100A gene (encoded by SEQ ID NO: 105 or 60%, 70%, 80%, 90%, or 95% identity thereto). Different combinations of these constructs for instance, as described by SEQ ID No. 1 to SEQ ID No. 31 and can be utilized for therapeutic purposes. A vector can be modular. A modular vector can allow for the replacement of one gene or nucleic acid segment for a different gene or nucleic acid segment, for example, using restriction enzyme digestion.

Multigenic expression of genes and proteins of interest can be mediated by a vector. Any vector system can be used including, but not limited to, plasmid vectors, retroviral vectors, lentiviral vectors, adenovirus vectors, poxvirus vectors, herpesvirus vectors, adeno-associated virus vectors, etc. Plasmid vectors are advantageous in avoiding host anti-viral immune responses. Plasmids can be episomal and non-integrating, which can reduce the risk of insertional mutagenesis compared with viral vectors. In some cases, an enhancer may be used in polynucleotide constructs of the invention. An enhancer can refer to a sequence that can function at no fixed distance from a transcription start site and can be either 5′ or 3′ to the transcription unit. Furthermore, enhancers can be within an intron as well as within the coding sequence itself. Enhancers are usually between 10 and 300 base pairs in length, and they function in cis. An enhancer can be from 10 bp to 50 bp, 50 bp to 100 bp, 100 bp to 150 bp, 150 bp to 200 bp, 200 bp to 250 bp, 250 bp to 300 bp. Enhancers can function to increase transcription from nearby promoters. Enhancers can also contain response elements that mediate the regulation of transcription. While many enhancer sequences are now known from mammalian genes (globin, elastase, albumin, fetoprotein and insulin), one may also use an enhancer from a eukaryotic cell virus for general expression. The choice of enhancer-promoter combination has a great impact on both the level and the duration of transgene expression. An enhancer can be a simian virus 40 (SV40) enhancer, a human immunodeficiency virus I (HIV-I) enhancer, ground squirrel hepatitis virus (GHV) enhancer, adenovirus enhancer, human prothrombin (hTHGB) enhancer, or human C2 complement gene (hC2) enhancer to name a few.

In some embodiments, an enhancer sequence can be used to increase expression of a gene. For example, a CMV enhancer can increase transgene expression under different cell-specific promoters and different cell types making it a broadly applicable tool to increase transgene expression levels.

In some cases, a mini-circle vector can be used. Minicircle vectors can differ from bacterial plasmid vectors in that they may lack an origin of replication, and may lack drug selection markers commonly found in bacterial plasmids, e.g. β-lactamase, tet, and the like. A minicircle may be substantially free of vector sequences other than the recombinase hybrid product sequence, and the sequence of interest, i.e. a transcribed sequence and regulatory sequences required for expression. In some cases, a dog bone vector may be utilized. A dog bone vector may be generated without the use of bacteria and may have reduced bacterial elements when compared to a plasmid. Reduced bacterial elements may reduce toxicity of vector introduction into a cell, into a subject, or a combination of both.

Some embodiments described herein comprise polynucleotides (and polypeptides encoded by said polynucleotides) which may be incorporated into vectors (including non-viral and viral vectors (including DNA and RNA/mRNA vectors, which may be linear or circular vectors)) wherein said polynucleotides comprise a construct configuration selected from the group consisting of:
a) 5′-(promoter)-S100-(linker)-SDF-(linker)-VEGF-3′;
b) 5′-(promoter)-S100-(linker)-VEGF-(linker)-SDF-3′;
c) 5′-(promoter)-S100A1-(linker)-SDF1-(linker)-VEGF-3′;
d) 5′-(promoter)-S100A1-(linker)-VEGF-(linker)-SDF1-3′;
e) 5′-(promoter)-S100A1-(linker)-SDF1-(linker)-VEGF165-3′;
f) 5′-(promoter)-S100A1-(linker)-VEGF165-(linker)-SDF1-3′;
g) 5′-(promoter)-S100A1-(linker)-SDF1α-(linker)-VEGF165-3′;
h) 5′-(promoter)-S100A1-(linker)-VEGF165-(linker)-SDF1α-3′;
i) 5′-(promoter)-S100A1-(linker)-SDF1-(linker)-VEGF191-3′;
j) 5′-(promoter)-S100A1-(linker)-VEGF191-(linker)-SDF1-3′;
k) 5′-(promoter)-S100A1-(linker)-SDF1α-(linker)-VEGF191-3′;
l) 5′-(promoter)-S100A1-(linker)-VEGF191-(linker)-SDF1α-3′;
m) 5′-(promoter)-S100-(fp2a)-SDF-(linker)-VEGF-3′;
n) 5′-(promoter)-S100-(fp2a)-VEGF-(linker)-SDF-3′;
o) 5′-(promoter)-S100A1-(fp2a)-SDF1-(linker)-VEGF-3′;
p) 5′-(promoter)-S100A1-(fp2a)-VEGF-(linker)-SDF1-3′;
q) 5′-(promoter)-S100A1-(fp2a)-SDF1-(linker)-VEGF165-3′;
r) 5′-(promoter)-S100A1-(fp2a)-VEGF165-(linker)-SDF1-3′;
s) 5′-(promoter)-S100A1-(fp2a)-SDF1α-(linker)-VEGF165-3′;
t) 5′-(promoter)-S100A1-(fp2a)-VEGF165-(linker)-SDF1α-3′;
u) 5′-(promoter)-S100A1-(fp2a)-SDF1-(linker)-VEGF191-3′;
v) 5′-(promoter)-S100A1-(fp2a)-VEGF191-(linker)-SDF1-3′;
w) 5′-(promoter)-S100A1-(fp2a)-SDF1α-(linker)-VEGF191-3′;
x) 5′-(promoter)-S100A1-(fp2a)-VEGF191-(linker)-SDF1α-3′;
y) 5′-(promoter)-S100-(fp2a)-SDF-(fp2a)-VEGF-3′;
z) 5′-(promoter)-S100-(fp2a)-VEGF-(fp2a)-SDF-3′;
aa) 5′-(promoter)-S100A1-(fp2a)-SDF1-(fp2a)-VEGF-3′;
ab) 5′-(promoter)-S100A1-(fp2a)-VEGF-(fp2a)-SDF1-3′;
ac) 5′-(promoter)-S100A1-(fp2a)-SDF1-(fp2a)-VEGF165-3′;
ad) 5′-(promoter)-S100A1-(fp2a)-VEGF165-(fp2a)-SDF1-3′;
ae) 5′-(promoter)-S100A1-(fp2a)-SDF1α-(fp2a)-VEGF165-3′;
at) 5′-(promoter)-S100A1-(fp2a)-VEGF165-(fp2a)-SDF1α-3′;
ag) 5′-(promoter)-S100A1-(fp2a)-SDF1-(fp2a)-VEGF191-3′;
ah) 5′-(promoter)-S100A1-(fp2a)-VEGF191-(fp2a)-SDF1-3′;
ai) 5′-(promoter)-S100A1-(fp2a)-SDF1α-(fp2a)-VEGF191-3′;
aj) 5′-(promoter)-S100A1-(fp2a)-VEGF191-(fp2a)-SDF1α-3′;
ak) 5′-(promoter)-S100-(fp2a)-SDF-(fmdv)-VEGF-3′;
al) 5′-(promoter)-S100-(fp2a)-VEGF-(fmdv)-SDF-3′;
am) 5′-(promoter)-S100A1-(fp2a)-SDF1-(fmdv)-VEGF-3′;
an) 5′-(promoter)-S100A1-(fp2a)-VEGF-(fmdv)-SDF1-3′;
ao) 5′-(promoter)-S100A1-(fp2a)-SDF1-(fmdv)-VEGF165-3′;
ap) 5′-(promoter)-S100A1-(fp2a)-VEGF165-(fmdv)-SDF1-3′;
aq) 5′-(promoter)-S100A1-(fp2a)-SDF1α-(fmdv)-VEGF165-3′;
ar) 5′-(promoter)-S100A1-(fp2a)-VEGF165-(fmdv)-SDF1α-3′;
as) 5′-(promoter)-S100A1-(fp2a)-SDF1-(fmdv)-VEGF191-3′;
at) 5′-(promoter)-S100A1-(fp2a)-VEGF191-(fmdv)-SDF1-3′;
au) 5′-(promoter)-S100A1-(fp2a)-SDF1α-(fmdv)-VEGF191-3′;
av) 5′-(promoter)-S100A1-(fp2a)-VEGF191-(fmdv)-SDF1α-3′;
aw) 5′-(promoter)-S100-(fp2a)-SDF-(p2a)-VEGF-3′;
ax) 5′-(promoter)-S100-(fp2a)-VEGF-(p2a)-SDF-3′;
ay) 5′-(promoter)-S100A1-(fp2a)-SDF1-(p2a)-VEGF-3′;
az) 5′-(promoter)-S100A1-(fp2a)-VEGF-(p2a)-SDF1-3′;
ba) 5′-(promoter)-S100A1-(fp2a)-SDF1-(p2a)-VEGF165-3′;
bb) 5′-(promoter)-S100A1-(fp2a)-VEGF165-(p2a)-SDF1-3′;
be) 5′-(promoter)-S100A1-(fp2a)-SDF1α-(p2a)-VEGF165-3′;
db) 5′-(promoter)-S100A1-(fp2a)-VEGF165-(p2a)-SDF1α-3′;
be) 5′-(promoter)-S100A1-(fp2a)-SDF1-(p2a)-VEGF191-3′;
bf) 5′-(promoter)-S100A1-(fp2a)-VEGF191-(p2a)-SDF1-3′;
bg) 5′-(promoter)-S100A1-(fp2a)-SDF1α-(p2a)-VEGF191-3′;
bh) 5′-(promoter)-S100A1-(fp2a)-VEGF191-(p2a)-SDF1α-3′, bi) 5′-(promoter)-S100-(fp2a)-SDF-(GSG-p2a)-VEGF-3′;
bk) 5′-(promoter)-S100-(fp2a)-VEGF-(GSG-p2a)-SDF-3′;
bl) 5′-(promoter)-S100A1-(fp2a)-SDF1-(GSG-p2a)-VEGF-3′;
bm) 5′-(promoter)-S100A1-(fp2a)-VEGF-(GSG-p2a)-SDF1-3′;
bn) 5′-(promoter)-S100A1-(fp2a)-SDF1-(GSG-p2a)-VEGF165-3′;
bo) 5′-(promoter)-S100A1-(fp2a)-VEGF165-(GSG-p2a)-SDF1-3′;
bp) 5′-(promoter)-S100A1-(fp2a)-SDF1α-(GSG-p2a)-VEGF165-3′;
bq) 5′-(promoter)-S100A1-(fp2a)-VEGF165-(GSG-p2a)-SDF1α-3′;
br) 5′-(promoter)-S100A1-(fp2a)-SDF1-(GSG-p2a)-VEGF191-3′;
bs) 5′-(promoter)-S100A1-(fp2a)-VEGF191-(GSG-p2a)-SDF1-3′;
bt) 5′-(promoter)-S100A1-(fp2a)-SDF1α-(GSG-p2a)-VEGF191-3′; and,
bu) 5′-(promoter)-S100A1-(fp2a)-VEGF191-(GSG-p2a)-SDF1α-3′;
wherein “promoter” is any transcriptional promoter sequence;
wherein “S100” encodes any polypeptide, isoform, or fragment thereof in the S100 family of proteins;
wherein “SDF” encodes any polypeptide, isoform, or fragment thereof in the SDF family of proteins; wherein “VEGF” encodes any polypeptide, isoform, or fragment thereof in the VEGF family of proteins;
wherein the parenthetical symbols “(” and “)” indicate any number of additional nucleotides (nts) (from 0 to 1000 nts) whether coding sequences or non-coding sequences;
wherein “linker” indicates any linker useful for expression of adjacent biologically active polypeptides;
wherein “S100A1” is at least 65%, 70%, 75%, 80%, 85%, 90%, 95%, 99% identical or is 100% identical to the nucleotide sequence of SEQ ID NO: 105 or to the amino acid sequence of SEQ ID NO: 104, in the, respective, context of polynucleotide or polypeptide sequences;
wherein “SDF-1α” is at least 65%, 70%, 75%, 80%, 85%, 90%, 95%, 99% identical or is 100% identical to the nucleotide sequence of SEQ ID NO: 103 or to the amino acid sequence of SEQ ID NO: 102, in the, respective, context of polynucleotide or polypeptide sequences;
wherein “VEGF165” is at least 65%, 70%, 75%, 80%, 85%, 90%, 95%, 99% identical or is 100% identical to the nucleotide sequence of SEQ ID NO: 127 or to the amino acid sequence of SEQ ID NO: 126, in the, respective, context of polynucleotide or polypeptide sequences;
wherein “VEGF191” is at least 65%, 70%, 75%, 80%, 85%, 90%, 95%, 99% identical or is 100% identical to the nucleotide sequence of SEQ ID NO: 107 or to the amino acid sequence of SEQ ID NO: 106, in the, respective, context of polynucleotide or polypeptide sequences;
wherein “fp2a” is at least 40%, 45%, 50%, 55%, 65%, 70%, 75%, 80%, 85%, 90%, 95% identical or is 100% identical to the nucleotide sequence of SEQ ID NO: 41 or to the amino acid sequence of SEQ ID NO: 40, in the, respective, context of polynucleotide or polypeptide sequences;
wherein “fmdv” is at least 40%, 45%, 50%, 55%, 65%, 70%, 75%, 80%, 85%, 90%, 95% identical or is 100% identical to the nucleotide sequence of SEQ ID NO: 35 or to the amino acid sequence of SEQ ID NO: 34, in the, respective, context of polynucleotide or polypeptide sequences;
wherein “p2a” is at least 40%, 45%, 50%, 55%, 65%, 70%, 75%, 80%, 85%, 90%, 95% identical or is 100% identical to the nucleotide sequence of SEQ ID NO: 37 or to the amino acid sequence of SEQ ID NO: 36, in the, respective, context of polynucleotide or polypeptide sequences; and,
wherein “GSG-p2a” is at least 40%, 45%, 50%, 55%, 65%, 70%, 75%, 80%, 85%, 90%, 95% identical or is 100% identical to the nucleotide sequence of SEQ ID NO: 39 or to the amino acid sequence of SEQ ID NO: 40, in the, respective, context of polynucleotide or polypeptide sequences. In some embodiments, lead candidate therapeutic polynucleotides of the invention comprise polynucleotides (and polypeptides encoded by said polynucleotides) which may be incorporated into vectors (including non-viral and viral vectors (including DNA and RNA/mRNA vectors, which may be linear or circular vectors)) wherein said polynucleotides comprise a construct configuration selected from the group consisting of:
a) 5′-(promoter)-S100A1-(linker)-SDF1α-(linker)-VEGF191-3′;
b) 5′-(promoter)-S100A1-(linker)-VEGF191-(linker)-SDF1α-3′;
c) 5′-(promoter)-S100A1-(linker)-SDF1α-(linker)-VEGF165-3′;
d) 5′-(promoter)-S100A1-(linker)-VEGF165-(linker)-SDF1α-3′;
e) 5′-(promoter)-S100A1-(fp2a)-SDF1α-(linker)-VEGF191-3′;
f) 5′-(promoter)-S100A1-(fp2a)-VEGF191-(linker)-SDF1α-3′;
g) 5′-(promoter)-S100A1-(fp2a)-SDF1α-(linker)-VEGF165-3′;
h) 5′-(promoter)-S100A1-(fp2a)-VEGF165-(linker)-SDF1α-3′;
i) 5′-(promoter)-S100A1-(fp2a)-SDF1α-(fp2a)-VEGF191-3′;
j) 5′-(promoter)-S100A1-(fp2a)-VEGF191-(fp2a)-SDF1α-3′;
k) 5′-(promoter)-S100A1-(fp2a)-SDF1α-(fp2a)-VEGF165-3′;
l) 5′-(promoter)-S100A1-(fp2a)-VEGF165-(fp2a)-SDF1α-3′;
m) 5′-(promoter)-S100A1-(fp2a)-SDF1α-(fmdv)-VEGF191-3′;
n) 5′-(promoter)-S100A1-(fp2a)-VEGF191-(fmdv)-SDF1α-3′;
o) 5′-(promoter)-S100A1-(fp2a)-SDF1α-(fmdv)-VEGF165-3′;
p) 5′-(promoter)-S100A1-(fp2a)-VEGF165-(fmdv)-SDF1α-3′;
q) 5′-(promoter)-S100A1-(fp2a)-SDF1α-(p2a)-VEGF191-3′;
r) 5′-(promoter)-S100A1-(fp2a)-VEGF191-(p2a)-SDF1α-3′;
s) 5′-(promoter)-S100A1-(fp2a)-SDF1α-(p2a)-VEGF165-3′;
t) 5′-(promoter)-S100A1-(fp2a)-VEGF165-(p2a)-SDF1α-3′;
u) 5′-(promoter)-S100A1-(fp2a)-SDF1α-(GSG-p2a)-VEGF191-3′;
v) 5′-(promoter)-S100A1-(fp2a)-VEGF191-(GSG-p2a)-SDF1α-3′;
w) 5′-(promoter)-S100A1-(fp2a)-SDF1α-(GSG-p2a)-VEGF165-3′; and,
x) 5′-(promoter)-S100A1-(fp2a)-VEGF165-(GSG-p2a)-SDF1α-3′;
wherein “promoter” is any transcriptional promoter sequence;
wherein “S100” encodes any polypeptide, isoform, or fragment thereof in the S100 family or proteins;
wherein “SDF” encodes any polypeptide, isoform, or fragment thereof in the SDF family of proteins;
wherein “VEGF” encodes any polypeptide, isoform, or fragment thereof in the VEGF family of proteins;
wherein the parenthetical symbols “(” and “)” indicate any number of additional nucleotides (nts) (from 0 to 1000 nts) whether coding sequences or non-coding sequences;
wherein “linker” indicates any linker useful for expression of adjacent biologically active polypeptides;
wherein “S100A1” is at least 65%, 70%, 75%, 80%, 85%, 90%, 95%, 99% identical or is 100% identical to the nucleotide sequence of SEQ ID NO: 105 or to the amino acid sequence of SEQ ID NO: 104, in the, respective, context of polynucleotide or polypeptide sequences;
wherein “SDF-1α” is at least 65%, 70%, 75%, 80%, 85%, 90%, 95%, 99% identical or is 100% identical to the nucleotide sequence of SEQ ID NO: 103 or to the amino acid sequence of SEQ ID NO: 102, in the, respective, context of polynucleotide or polypeptide sequences;
wherein “VEGF165” is at least 65%, 70%, 75%, 80%, 85%, 90%, 95%, 99% identical or is 100% identical to the nucleotide sequence of SEQ ID NO: 127 or to the amino acid sequence of SEQ ID NO: 126, in the, respective, context of polynucleotide or polypeptide sequences;
wherein “VEGF191” is at least 65%, 70%, 75%, 80%, 85%, 90%, 95%, 99% identical or is 100% identical to the nucleotide sequence of SEQ ID NO: 107 or to the amino acid sequence of SEQ ID NO: 106, in the, respective, context of polynucleotide or polypeptide sequences;
wherein “fp2a” is at least 40%, 45%, 50%, 55%, 65%, 70%, 75%, 80%, 85%, 90%, 95% identical or is 100% identical to the nucleotide sequence of SEQ ID NO: 41 or to the amino acid sequence of SEQ ID NO: 40, in the, respective, context of polynucleotide or polypeptide sequences;
wherein “fmdv” is at least 40%, 45%, 50%, 55%, 65%, 70%, 75%, 80%, 85%, 90%, 95% identical or is 100% identical to the nucleotide sequence of SEQ ID NO: 35 or to the amino acid sequence of SEQ ID NO: 34, in the, respective, context of polynucleotide or polypeptide sequences;
wherein “p2a” is at least 40%, 45%, 50%, 55%, 65%, 70%, 75%, 80%, 85%, 90%, 95% identical or is 100% identical to the nucleotide sequence of SEQ ID NO: 37 or to the amino acid sequence of SEQ ID NO: 36, in the, respective, context of polynucleotide or polypeptide sequences; and,
wherein “GSG-p2a” is at least 40%, 45%, 50%, 55%, 65%, 70%, 75%, 80%, 85%, 90%, 95% identical or is 100% identical to the nucleotide sequence of SEQ ID NO: 39 or to the amino acid sequence of SEQ ID NO: 40, in the, respective, context of polynucleotide or polypeptide sequences.

In a preferred embodiment, compositions of the present invention may be used to treat congestive heart failure (e.g., compositions of the present invention may be used to treat congestive heart failure in patients with implanted destination left ventricular assist devices (LVAD)).

In a preferred embodiment, compositions of the present invention are administered to patients through retrograde coronary sinus infusion (RCSI).

In one embodiment, compositions of the present invention are administered to animal models (for example, but not limited to, pigs (porcine model)) wherein retrograde coronary sinus infusion (RCSI) is used to study an animal model of myocardial ischemia.

In certain embodiments, the functionality of each effector in pXoX was verified in transfected healthy and diseased patient-induced pluripotent stem cell (iPSC)-derived cardiomyocytes using effector-specific assays. In particular, S100A1 demonstrated an improvement in contractile properties. SDF-1α was shown to induce the CXCR4 dependent migration of Jurkat cells and peripheral blood lymphocytes (PBLs). VEGF165 demonstrated an increase in proliferation of human umbilical endothelial cells (HUVECs).

In another embodiment, transfection of pXoX in vitro safety testing in healthy and diseased patient-induced pluripotent stem cell (iPSC)-derived cardiomyocytes did not demonstrate risks for adverse effects on cardiac conduction, based on assessments of changes in Field Potential Duration (MEA), which correlate closely with effects on QT intervals of the electrocardiogram (ECG).

In another embodiment, transfection of pXoX restored beat rate, contraction duration and contraction rate in dilated cardiomyopathy patient iPSC-CMs to levels comparable to those seen in iPSC-CM from a healthy phenotype. In a preferred embodiment, the best improvements in contractile properties were observed for the pXoX triple effector plasmid, relative to single or dual effector plasmids.

In certain embodiments, vectors and polynucleotide constructs of the invention comprise dual-gene (double-gene) sequences (and polypeptides encoded by same) such as those described in Table 1.

TABLE 1
Vector Features - Dual-Gene (Double-Gene) Constructs

| | | Orientation | |
|---|---|---|---|
| SEQ ID No. | Name* | (N to C terminus) | Linker |
| 1 | CAG-S100A1-fmdv-SDF1-NXB-V2 | S100A1-SDF1 | fmdv |
| 2 | CAG-SDF1-fmdv-S100A1-NXB-V2 | SDF1-S100A1 | fmdv |
| 3 | CAG-SDFl-fmdv-VEGF191-NXB-V2 | SDF-VEGF191 | fmdv |
| 4 | CAG-VEGF191-fmdv-SDF1-NXB-V2 | VEGF191-SDF1 | fmdv |
| 5 | CAG-S100A1-p2a-SDF1-NXB-V2 | S100A1-SDF1 | p2a |
| 6 | CAG-SDF1-p2a-S100A1-NXB-V2 | SDF1-S100A1 | p2a |
| 7 | CAG-SDF1-p2a-VEGF191-NXB-V2 | SDF1-VEGF191 | p2a |
| 8 | CAG-VEGF-191-p2a-SDF1-NXB-V2 | VEGF191-SDF1 | p2a |
| 9 | CAG-S100A1-fp2a-SDF1-NXB-V2 | S100A1-SDF1 | fp2a |
| 10 | CAG-SDF1-fp2a-S100A1-NXB-V2 | SDF1-S100A1 | fp2a |
| 11 | CAG-SDF1-fp2a-VEGF191-NXB-V2 | SDF1-VEGF191 | fp2a |
| 12 | CAG-VEGF191-fp2a-SDF-1NXB-V2 | VEGF191-SDF1 | fp2a |
| 13 | CAG-VEGF191-fp2a-S100A1NXB-V2 | VEGF191-S100A1 | fp2a |
| 14 | CAG-S100A1-fmdv-VEGF191-NXB-V2 | S100A1-VEGF191 | fmdv |
| 15 | CAG-VEGF191-fmdv-S100A1-NXB-V2 | VEGF191-S100A1 | fmdv |
| 16 | CAG-S100A1-p2a-VEGF191-NXB-V2 | S100A1-VEGF191 | p2a |
| 17 | CAG-VEGF191-p2a-S100A1-NXB-V2 | VEGF191-S100A1 | p2a |
| 18 | CAG-S100A1-fp2a-VEGF191-NXB-V2 | S100A1-VEGF191 | fp2a |
| 19 | IGE-641pCAGCAG(1660043)GAPDH(5883) | S100A1-SDF1 | fmdv |
| | Kozak1744359F3R(26319) | | |
| 20 | IGE-642 pCAG CAG(1660043)GAPDH(5883) | SDF1-S100A1 | fmdv |
| | Kozak1744365F3R(26319) | | |
| 21 | IGE-643 pCAG CAG(1660043)GAPDH(5883) | SDF1-VEGF191 | fmdv |
| | Kozak+1744369F3R(26319) | | |
| 22 | IGE-644 pCAG CAG(1660043)GAPDH(5883) | VEGF191-SDF1 | fmdv |
| | Kozak 1744395F3R(26319) | | |
| 23 | IG E-645 pcag | S100A1-SDF1 | p2a |
| | CAG(1660043)_GAPDH(5883)_Kozak+1744405_F3R(26319) | | |
| 24 | IGE- | SDF1-S100A1 | p2a |
| | 646_pCAG_CAG(1660043)_GAPDH(5883)_Kozak+1744407_F3R(26319) | | |
| 25 | IGE- | SDF1-VEGF191 | p2a |
| | 647_pCAG_CAG(1660043)_GAPDH(5883)_Kozak+1744420_F3R(26319) | | |
| 26 | IGE- | VEGF191-SDF1 | p2a |
| | 648_pCAG_CAG(1660043)_GAPDH(5883)_Kozak+1744423_F3R(26319) | | |
| 27 | IGE- | S100A1-SDF1 | fp2a |
| | 649_pCAG_CAG(1660043)_GAPDH(5883)_Kozak+1744427_F3R(26319) | | |
| 28 | IGE-650pCAG CAG(1660043)GAPDH(5883) | SDF1-S100A1 | fp2a |
| | Kozak1744430F3R(26319) | | |
| 29 | IGE-651 pCAG CAG(1660043)GAPDH(5883) | SDF1-VEGF191 | fp2a |
| | Kozak1744431F3R(26319) | | |
| 30 | IGE-652 pCAG CAG(1660043)GAPDH(5883) | VEGF191-SDF1 | fp2a |
| | Kozak1744432F3R(26319) | | |
| 31 | IGE-653 pCAG CAG(1660043)GAPDH(5883) | VEGF-S100A1 | fp2a |
| | Kozak1744433F3R(26319) | | |

*”CAG” and “NXB-V2” represent the CAG promoter plus 5′ and 3′ elements and the new X
vector backbone version-2 as found in constructs of the invention (such as in pXoX as indicated below):
Sequence of “CAG” and “NXB-V2” vector elements

| CAG_NXB-V2 | Nucleotide Positions in SEQ ID NO: 108 |
|---|---|
| NXB-V2 = Plasmid Backbone components: | 1-114 (114 bp); |
| | 3753-3852 (100 bp); |
| | 3853-4904 (1052 bp); |
| | 4905-4960 (56 bp); |
| | 4961-5755 (795 bp); and, |
| | 5756-6025 (270 bp) |
| CAG = CAG Promoter + 5′ and 3′ elements: | 115-1825 (1711 bp); |
| | 1841-1942 (102 bp); and |
| | 3266-3752 (487 bp) |

### Promoters

Promoters are a major cis-acting element within the vector genome design that can dictate the overall strength of expression as well as cell-specificity. A promotor can be a ubiquitous promotor, a cell-specific promotor, or a combination thereof. A ubiquitous or constitutive promoter can be a CMV, CBA (CAG and CBh), EF-1alpha, PKG, UBC, GUSB (hGBp), UCOE, to name a few. A promotor can be a promotor that has activity within a target cell of interest, e.g., cardiac promotor. In some cases a promotor is a CAG. In other cases a promoter is a constitutive promoter such as Ef1a. A promoter can be a human promotor, such as human Ef1a (hEf1a). A bidirectional promoter can also be used. For example, a constitutive bidirectional promoter can be a human cytomegalovirus promoter.

In some embodiments, such as those where a gene product polypeptide is secreted, ubiquitous expression in all or select cell types can desired. Constitutive promoters such as the human elongation factor 1α-subunit (EF1α), immediate-early cytomegalovirus (CMV), chicken 3-actin (CBA) and its derivative CAG, the p glucuronidase (GUSB), or ubiquitin C (UBC) can be used to promote expression in most tissues (Husain et al., 2009; Qin et al., 2010; Norrman et al., 2010). Generally, CBA and CAG promote the larger expression among the constitutive promoters (Xu et al., 2001; Yin et al., 2011); however, their size of ˜1.7 kbs in comparison to CMV (˜0.8 kbs) or EF1α (˜1.2 kbs) can limit its use in vectors with packaging constraints. In some cases, a GUSB or UBC promoter can provide ubiquitous gene expression with a smaller size of 378 bps and 403 bps, respectively, but they can be considerably weaker than a CMV or CBA promoter (Husain et al., 2009; Qin et al., 2010). Thus, modifications to constitutive promoters in order to reduce the size without affecting its expression have been pursued and examples such as the CBh (˜800 bps) and the miniCBA (˜800 bps) can promote expression comparable and even higher in selected tissues (Gray et al., 2011). In some cases “ubiquitous” promoters can be prone to silencing or promote differential expression strength in selected cell types (McCown et al., 1996; Klein et al., 1998; Gray et al., 2011).

Viral enhancers and promoters derived from cytomegalovirus (CMV), respiratory syncytial virus (RSV) and simian virus 40 (SV40) can be used to achieve high levels of expression in a range of mammalian cell and tissue types, such as in cardiomyocytes. Constitutive mammalian promoters, such as the human ubiquitin C (UBC) and the eukaryotic translation elongation factor 1 alpha 1 (EEF1A1) promoters can have more persistent expression. Tissue-specific promoters can be utilized to reduce off-target transgene expression. Numerous cis-acting sequences, including polyadenylation signals, introns and scaffold/matrix attachment regions (S/MARs) 52, can increase the level and persistence of transgene expression in some cases.

Compact DNA vectors that lack a bacterial backbone (e.g., minicircles) can maintain superior levels and duration of gene expression relative to full-length DNA plasmids. A dog bone vector may also be utilized. A dog bone vector can be generated without the use of bacteria. In some cases, a lipid-based DNA vectors can also be used in some cases, a transposon system can be utilized. For example, a transposition system can be or can be based on the recombinases phiC31, PiggyBac57, Sleeping Beauty, or combinations thereof.

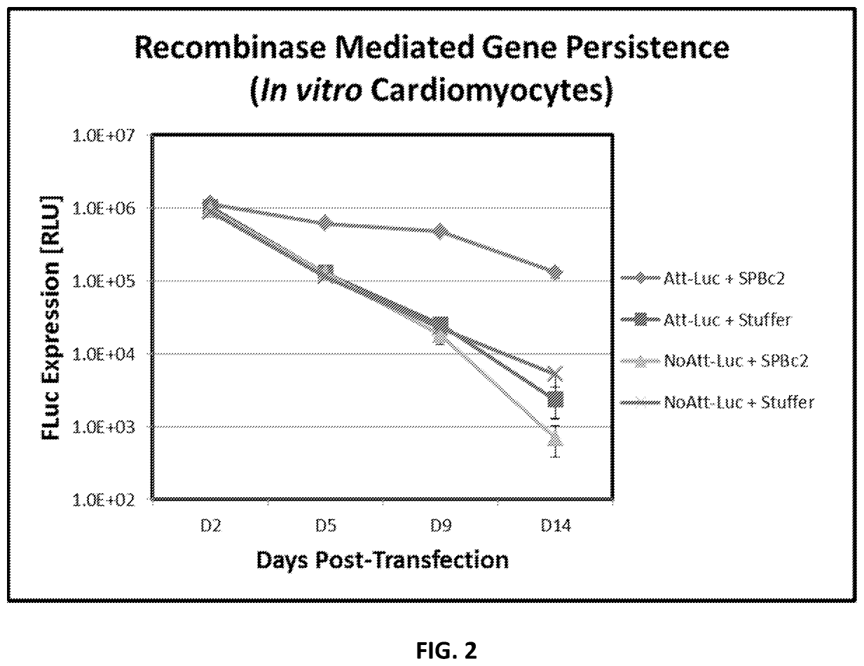

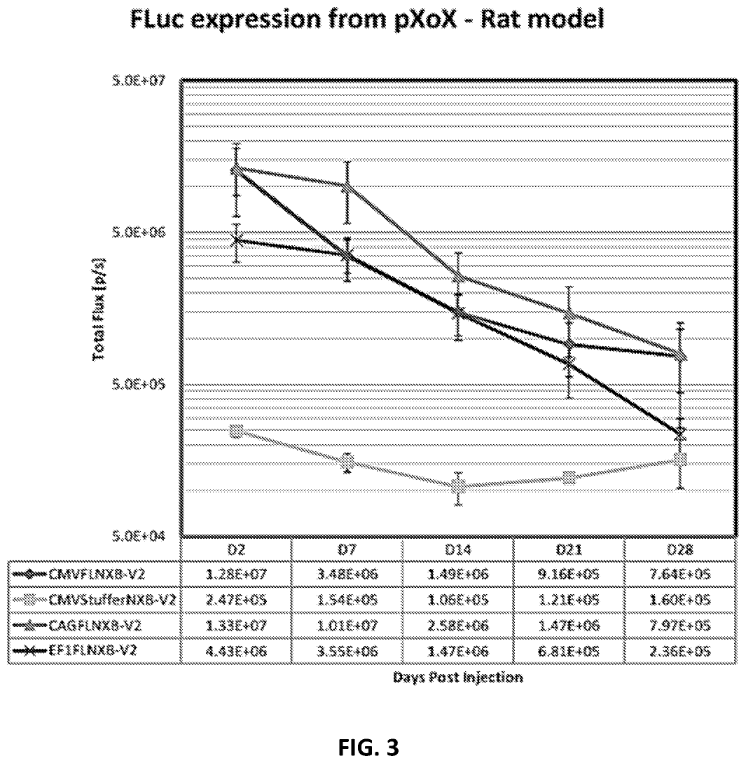

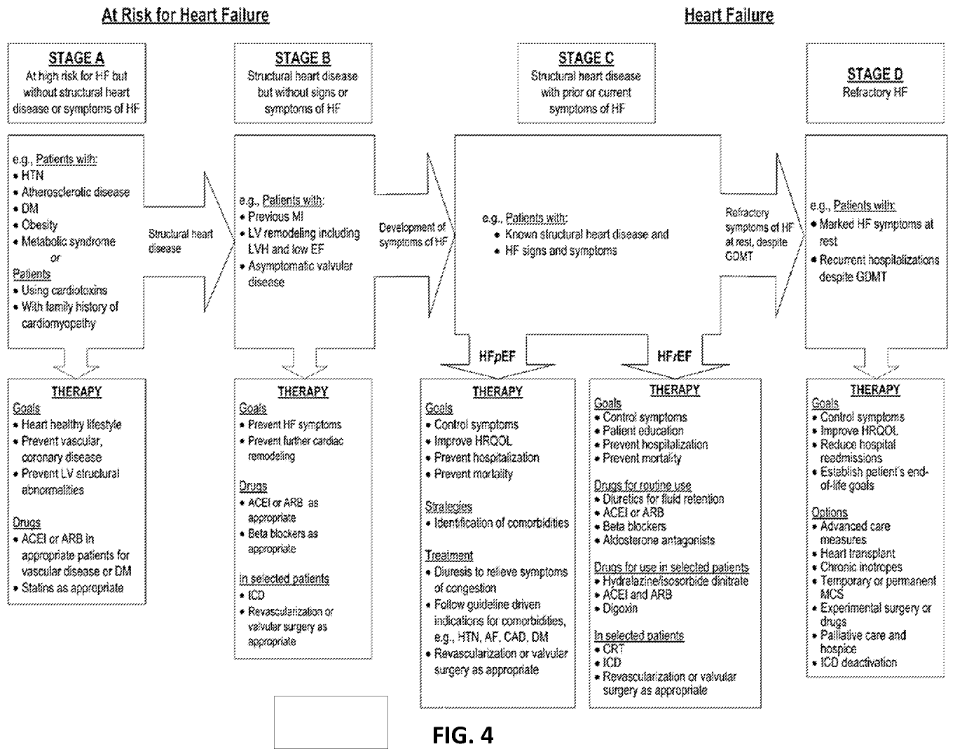

In some cases, cardiac gene effector vectors described herein can be integrated into a cell genome via use of recombinase, FIG. 2. A recombinase can be an att-site recombinase. In some cases, a recombinase can be selected from recombinases as described in: U.S. Pat. No. 9,034,650; US Pub. No. 2015/0275232; and, W02008/073154, each of which is incorporated by reference herein in its entirety.

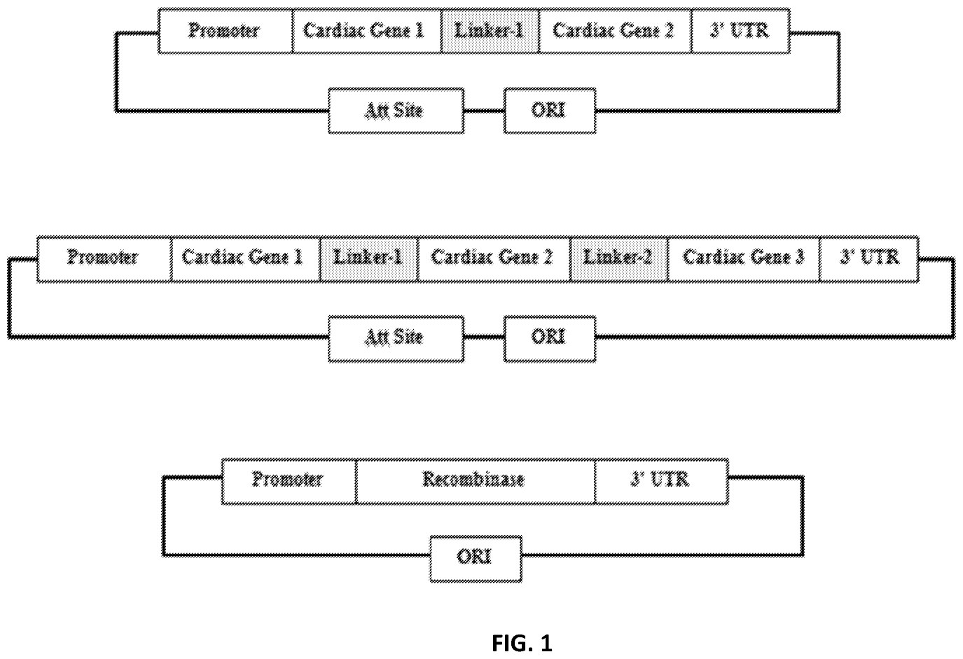

A recombinase can be introduced into a cell via an mRNA encoding a recombinase. In certain embodiments, integration of a recombinase into a cell genome can be prevented by introduction and expression of a recombinase via use of an mRNA encoding a recombinase. In some cases, an mRNA encoding a recombinase can be a synthetic or a cGMP-grade mRNA, FIG. 1.

In some cases, a recombinase can be introduced into a cell via an expression plasmid comprising a “suicide gene” and a gene encoding a recombinase. In certain embodiments a suicide gene and recombinase gene can be separated by an intervening internal ribosome entry site (IRES).

In some cases, a cell in which vectors can be introduced or genomically integrated (e.g., via recombinase) can be a primary cell (e.g., such as, but not limited to, a cardiomyocyte, FIG. 2). In some cases, a gene encoded within a vector can be genomically inserted at a targeted genomic location in a cell.

Internal ribosome entry site (IRES) elements can allow expression of multiple genes from one transcript (Mountford and Smith 1995). The term “IRES” as used herein can be intended to mean internal ribosomal entry site. In a vector comprising an IRES sequence, a first gene can be translated by a cap-dependent, ribosome scanning, mechanism with its own 5′-UTR, whereas translation of a subsequent gene can be accomplished by direct recruitment of a ribosome to an IRES in a cap-independent manner.

An IRES sequence can allow eukaryotic ribosomes to bind and begin translation without binding to a 5′ capped end. In certain cases, an IRES region can be derived from a virus, such as picornavirus, encephalomyocarditis virus, hepatitis C virus IRES sequence. In other cases, can IRES sequence can be derived from an encephalomyocarditis virus. The term “EMCV” or “encephalomyocarditis virus” as used herein refers to any member isolate or strain of the encephalomyocarditis virus species of the genus of the family Picornaviridae. Examples are: EMCV-R (Rueckert) strain virus, Columbia-SK virus. In some cases, a cellular IRES element, such as eukaryotic initiation factor 4G, immunoglobulin heavy chain binding protein, c-myc proto-oncogene, vascular endothelial growth factor, fibroblast growth factor-1 IRES, or any combination or modification thereof can be used. In some cases, a cellular IRES can have increased gene expression when compared to a viral IRES.

The term “CAP” or “cap” as used herein refers to a modified nucleotide, generally a 7-methyl guanosine, linked 3′ to 5′ (7meG-ppp-G), to the 5′ end of a eukaryotic mRNA, that serves as a required element in the normal translation initiation pathway during expression of protein from that mRNA.

An IRES sequence of viral, cellular or a combination thereof can be utilized in a vector. An IRES can be from encephalomyocarditis (EMCV) or poliovirus (PV).

In some cases, an IRES element is selected from a group consisting of Poliovirus (PV), Encephalomyelitis virus (EMCV), Foot-and-mouth disease virus (FMDV), Porcine teschovirus-1 (PTV-1), Aichivirus (AiV), Seneca Valley virus (SVV), Hepatitis C virus (HCV), Classical swine fever virus (CSFV), Human immunodeficiency virus-2 (HIV-2), Human immunodeficiency virus-1 (HIV-1), Moloney murine leukemia virus (MoMLV), Feline immunodeficiency virus (FIV), Mouse mammary tumor virus (MMTV), Human cytomegalovirus latency (pUL138), Epstein-Barr virus (EBNA-1), Herpes virus Marek's disease (MDV RLORF9), SV40 polycistronic 19S (SV40 19S), Rhopalosiphum padi virus (RhPV), Cricket paralysis virus (CrPV), Ectropis obliqua picorna-like virus (EoPV), Plautia stali intestine virus (PSIV), Triatoma virus (TrV), Bee paralysis dicistrovirus (IAPV, KBV), Black currant reversion virus (BRV), Pelargonium flower break virus (PFBV), Hibiscus chlorotic ringspot virus (HCRSV), Crucifer-infecting tobamovirus (CrTMV), Potato leaf roll polerovirus (PLRV), Tobacco etch virus (TEV), Giardiavirus (GLV), Leishmania RNA virus-1 (LRV-1), and combinations or modifications thereof.

In some cases, an IRES is selected from a group consisting of Apaf-1, XIAP, HIAP2/c-IAP1, DAP5, Bcl-2, c-myc, CAT-1, INR, Differentiation LEF-1, PDGF2, HIF-1a, VEGF, FGF2, BiP, BAG-1, CIRP, p53, SHMT1, PITSLREp58, CDK1, Rpr, hid, hsp70, grim, skl, Antennapedia, dFoxO, dInR, Adh-Adhr, HSP101, ADH, URE-2, GPR1, NCE102, YMR181a, MSN1, BOI, FLO8, GIC1, and any combination or modification thereof.

When an IRES element is included between two open reading frames (ORFs), initiation of translation can occur by a canonical 5′-m7GpppN cap-dependent mechanism in a first ORF and a cap-independent mechanism in a second ORF downstream of the IRES element.

In some cases, genes can be linked by an internal ribosomal entry site (IRES). An IRES can allow simultaneous expression of multiple genes. For example, an IRES sequence can permit production of multiple proteins from a single mRNA transcript. A ribosomes can bind to an IRES in a 5′-cap independent manner and initiate translation.

In some cases, an IRES sequence can be or can be about 500 base pairs. An IRES sequence can be from 300 base pairs to 1000 base pairs. For example, an IRES can be 300, 350, 400, 450, 500, 550, 600, 650, 700, 750, 800, 850, 900, 950, or 1000 base pairs long.

In some cases, expression of a downstream gene within a vector comprising an IRES sequence can be reduced. For example, a gene following an IRES sequence can have reduced expression over a gene preceding an IRES sequence. Reduced expression can be from 1% to 99% reduction over a preceding gene.

In some cases, a viral 2A sequence can be used. A 2A sequence can be derived from a picornaviral 2A sequence. A picornaviral 2A sequence can be selected from the group consisting of the Enteroviral 2A sequences, Rhinoviral 2A sequences, Cardioviral 2A sequences, Aphthoviral 2A sequences, Hepatoviral 2A sequences, Erboviral 2A sequences, Kobuviral 2A sequences, Teschoviral 2A sequences, and the Parechoviral 2A sequences.

2A elements can be shorter than IRES, having from 5 to 100 base pairs. In some cases, a 2A sequence can have 5, 10, 15, 20, 25, 30, 35, 40, 45, 50, 55, 60, 65, 70, 75, 80, 85, 90, or 100 base pairs in length. 2A linked genes can be expressed in one single open reading frame and “self-cleavage” can occur co-translationally between the last two amino acids, GP, at the C-terminus of the 2A polypeptide, giving rise to equal amounts of co-expressed proteins. In some cases, a polypeptide comprising a 2A sequence may not give rise to equal amounts of protein post cleavage. In some cases, a first protein may be 10%, 20%, 30%, 40%, 50%, 60%, 70%, 80%, 90%, or up to 100% greater in concentration when compared to a second protein. In some cases, a second protein may be 10%, 20%, 30%, 40%, 50%, 60%, 70%, 80%, 90%, or up to 100% greater in concentration when compared to a second protein.

A viral 2A sequence can be about 20 amino acids. In some cases, a viral 2A sequence can contain a consensus motif Asp-Val/Ile-Glu-X-Asn-Pro-Gly-Pro (SEQ ID NO: 117). A consensus motif sequence can act co-translationally. For example, formation of a normal peptide bond between a glycine and proline residue can be prevented, which can result in ribosomal skipping and, thereby, “cleavage” of a nascent polypeptide. This effect can produce multiple genes at equimolar levels.

A 2A peptide can allow translation of multiple proteins in a single open reading frame into a polypeptide that can be subsequently “cleaved” into individual polypeptide through a ribosome-skipping mechanism (Funston, Kallioinen et al. 2008). In some embodiments, a “2A” sequence can include: p2a, GSG-p2a, T2A, E2A, F2A, and BmCPV2A, BmIFV2A, and any combination thereof.

A vector can also include additional a 2A/furin sequence located between polycistronic genes to permit production of expression products originating from e.g., a second gene by enzymatic cleavage of a polypeptide product. In some cases additional linkers may be utilized to facilitate cleavage between multiple genes in a multigene vector.

In some cases, a vector can comprise an IRES sequence and a 2A sequence. In other cases, expression of multiple genes linked with 2A peptides can be facilitated by a spacer sequence (GSG) ahead of the 2A peptides. In some cases, constructs can combine a spacers, linkers, adaptors, promotors, or combinations thereof. For example, a construct can have a spacer (SGSG (SEQ ID NO: 118) or GSG) and furin linker (RAKR (SEQ ID NO: 32)) cleavage site with different 2A peptides. A spacer can be an I-Ceui (intron encoding endonuclease). In certain embodiments two or more of the cardiac effector genes are separated by an intervening internal ribosome entry site (IRES).

### Polynucleotide Linkers

In embodiments described herein, a polynucleotide linker can be utilized in a polynucleotide described herein. A polynucleotide linker can be a double-stranded segment of DNA containing desired restriction sites that may be added to create end structures that are compatible with a vector comprising a polynucleotide described herein. In some cases, a polynucleotide linker can be useful for modifying vectors comprising polynucleotides described herein. For example, a vector modification comprising a polynucleotide linker can be a change in a multiple cloning site, or the addition of a poly-histidine tail. Polynucleotide linkers can also be used to adapt the ends of blunt insert DNA for cloning into a vector cleaved with a restriction enzyme with cohesive end termini. The use of polynucleotide linkers can be more efficient than a blunt ligation into a vector and can provide a method of releasing an insert from a vector in downstream applications. In some cases an insert can be a polynucleotide sequence encoding polypeptides useful for therapeutic applications (e.g., SDF1 polypeptide, a S100A1 polypeptide, a VEGF polypeptide and fragments and variants thereof).

A polynucleotide linker can be an oligomer. For example, a linker can be from about 5 to 20 nucleotides in length. A polynucleotide linker can be or can be about from 5, 6, 7, 8, 9, 10, 11, 12, 13, 14, 15, 16, 17, 18, 19, or 20 nucleotides in length. A polynucleotide linker can be a DNA double strand, single strand, or a combination thereof. In some cases, a linker can be RNA. A polynucleotide linker can be ligated into a vector comprising a polynucleotide described herein by a T4 ligase in some cases. To facilitate a ligation an excess of polynucleotide linkers can be added to a composition comprising an insert and a vector. In some cases, an insert and vector are pre-treated before a linker is introduced. For example, pre-treatment with a methylase can prevent unwanted cleavage of insert DNA.

### Polypeptide Linkers

In certain embodiments, two or more polypeptides encoded by a polynucleotide described herein can be separated by an intervening sequence encoding a linker polypeptide. In certain cases, the linker is a cleavage-susceptible linker. In some embodiments, polypeptides of interest are expressed as fusion proteins linked by a cleavage-susceptible linker polypeptide. In certain embodiments, cleavage-susceptible linker polypeptide(s) can be any one or two of: Furinlink, fmdv, p2a, GSG-p2a, and/or fp2a described below. In some cases, a linker is APVKQGSG (SEQ ID NO: 119).

In certain cases, a linker polypeptide can comprise an amino acid sequence “RAKR” (SEQ ID NO: 32). In certain cases, a Furin linker polypeptide can be encoded by a polynucleotide sequence polynucleotide sequence comprising “CGTGCAAAGCGT.” (SEQ ID NO: 33).

In certain cases, a linker polypeptide can be a linker comprising a sequence disclosed in the table below:

TABLE 2
Linker amino acid sequences and
polynucleotide sequences

| SEQ | Linker | Sequence(N- to C- terminus |
|---|---|---|
| ID No. | Name | or 5′ to 3′ as applicable) |
| 32 | Furinlink1 | RAKR |
| 33 | Furinlink1 | CGTGCAAAGCGT |
| 34 | fmdv | RAKRAPVKQTLNFDLLKLAGDVESNPGP |
| 35 | fmdv | AGAGCCAAGAGGGCACCGGTGAAACAGAC |
| | | TTTGAATTTTGACCTTCTGAAGTTGGCAG |
| | | GAGACGTTGAGTCCAACCCTGGGCCC |
| 36 | p2a | ATNFSLLKQAGDVEENPGP |
| 37 | p2a | GCTACTAACTTCAGCCTGCTGAAGCAGGC |
| | | TGGAGACGTGGAGGAGAACCCTGGACCT |
| 38 | GSG-p2a | GSGATNFSLLKQAGDVEENPGP |
| 39 | GSG-p2a | GGAAGCGGAGCTACTAACTTCAGCCTGCT |
| | | GAAGCAGGCTGGAGACGTGGAGGAGAACC |
| | | CTGGACCT |
| 40 | fp2a | RAKRAPVKQGSGATNFSLLKQAGDVEENP |
| | | GP |
| 41 | fp2a | CGTGCAAAGCGTGCACCGGTGAAACAGGG |
| | | AAGCGGAGCTACTAACTTCAGCCTGCTGA |
| | | AGCAGGCTGGAGACGTGGAGGAGAACCCT |
| | | GGACCT |

In some embodiments, a linker can be utilized in a polynucleotide described herein. A linker can be a flexible linker, a rigid linker, an in vivo cleavable linker, or any combination thereof. In some cases, a linker may link a functional domains together (as in flexible and rigid linkers) or releasing free functional domain in vivo as in in vivo cleavable linkers.

Linkers may improve biological activity, increase expression yield, and achieving desirable pharmacokinetic profiles. A linker can also comprise hydrazone, peptide, disulfide, or thioesther.

In some cases, a linker sequence described herein can include a flexible linker. Flexible linkers can be applied when a joined domain requires a certain degree of movement or interaction.

Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n (SEQ ID NO: 120). By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers included in linker sequences described herein, can be rich in small or polar amino acids such as Gly and Ser to provide good flexibility and solubility. Flexible linkers can be suitable choices when certain movements or interactions are desired for fusion protein domains. In addition, although flexible linkers may not have rigid structures, they can serve as a passive linker to keep a distance between functional domains. The length of a flexible linkers can be adjusted to allow for proper folding or to achieve optimal biological activity of the fusion proteins.

A linker described herein can further include a rigid linker in some cases. A rigid linker may be utilized to maintain a fixed distance between domains of a polypeptide. Examples of rigid linkers can be: Alpha helix-forming linkers, Pro-rich sequence, (XP)n (SEQ ID NO: 121), X-Pro backbone, A(EAAAK)nA (n=2-5)(SEQ ID NO: 122), to name a few. Rigid linkers can exhibit relatively stiff structures by adopting α-helical structures or by containing multiple Pro residues in some cases.

A linker described herein can be cleavable in some cases. In other cases a linker is not cleavable. Linkers that are not cleavable may covalently join functional domains together to act as one molecule throughout an in vivo processes or an ex vivo process. A linker can also be cleavable in vivo. A cleavable linker can be introduced to release free functional domains in vivo. A cleavable linker can be cleaved by the presence of reducing reagents, proteases, to name a few. For example, a reduction of a disulfide bond may be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein may also be carried out by proteases that can be expressed in vivo under pathological conditions (e.g. cancer or inflammation), in specific cells or tissues, or constrained within certain cellular compartments. In some cases, a cleavable linker may allow for targeted cleavage. For example, the specificity of many proteases can offer slower cleavage of a linker in constrained compartments. A cleavable linker can also comprise hydrazone, peptides, disulfide, or thioesther. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation.

In certain embodiments, an fmdv linker polypeptide comprises a sequence that can be at least about 45%, 50%, 55%, 60%, 65%, 70%, 75%, 80%, 85%, 90%, 95%, 97%, 98%, 99% or 100% identical to SEQ ID NO. 34. In certain embodiments, an fmdv linker polypeptide is one or more of the linkers encoded in a single vector linking two or more fusion proteins. In certain cases, an fmdv linker polypeptide can be encoded by a polynucleotide open-reading frame (ORF) nucleic acid sequence. In some cases, an ORF encoding fmdv comprises or consists of a sequence of SEQ ID NO. 35. In certain embodiments, a polynucleotide encoding fmdv is at least 45%, 50%, 55%, 60%, 65%, 70%, 75%, 80%, 85%, 90%, 95%, 97%, 98%, 99% or 100% identical to SEQ ID NO. 35.

In certain cases, a linker polypeptide can be a “p2a” linker. In certain embodiments, a p2a polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 36. In certain embodiments, the p2a linker polypeptide can be one or more of the linkers encoded in a single vector linking two or more fusion proteins. In some cases, a p2a linker polypeptide can be encoded by a polynucleotide open-reading frame (ORF) nucleic acid sequence. In certain embodiments, an ORF encoding p2a comprises or consists of the sequence of SEQ ID NO.37. In certain cases, a polynucleotide encoding p2a can be or can be about at least 45%, 50%, 55%, 60%, 65%, 70%, 75%, 80%, 85%, 90%, 95%, 97%, 98%, 99% or 100% identical to SEQ ID NO: 37.

In some cases, a linker polypeptide can be a “GSG-p2a” linker. In certain embodiments, a GSG-p2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 38. In certain embodiments, a GSG-p2a linker polypeptide can be one or more of the linkers encoded in a single vector linking two or more fusion proteins. In some cases, a GSG-p2a linker polypeptide can be encoded by a polynucleotide open-reading frame (ORF) nucleic acid sequence. An ORF encoding GSG-p2a can comprises the sequence of SEQ ID NO.39. In some cases, a polynucleotide encoding GSG-p2a can be or can be about at least 45%, 50%, 55%, 60%, 65%, 70%, 75%, 80%, 85%, 90%, 95%, 97%, 98%, 99% or 100% identical to SEQ ID NO: 39.

A linker polypeptide can be an “fp2a” linker as provided herein. In certain embodiments, a fp2a linker polypeptide can comprise a sequence that can be about at least 45%, 50%, 55%, 60%, 65%, 70%, 75%, 80%, 85%, 90%, 95%, 97%, 98%, 99% or 100% identical to SEQ ID NO: 40. In certain cases, an fp2a linker polypeptide can be one or more of the linkers encoded in a single vector linking two or more fusion proteins. In some cases, a fp2a linker polypeptide can be encoded by a polynucleotide open-reading frame (ORF) nucleic acid sequence. In certain embodiments, a polynucleotide encoding an fp2a linker can be or can be about at least 45%, 50%, 55%, 60%, 65%, 70%, 75%, 80%, 85%, 90%, 95%, 97%, 98%, 99% or 100% identical to SEQ ID NO: 41.

In some cases, a linker can be engineered. For example, a linker can be designed to comprise chemical characteristics such as hydrophobicity. In some cases, at least two linker sequences can produce the same protein. A sequence can be or can be about 45%, 50%, 55%, 60%, 65%, 70%, 75%, 80%, 85%, 90%, 95%, 96%, 97%, 98%, 99%, or 100% identical to a sequence of SEQ ID NO: 42 to SEQ ID NO:101. In other cases, multiple linkers can be used in a vector. For example, genes of interest (e.g., S100A1, SDF-1α, and VEGF165) can be separated by at least two linkers, as shown in FIG. 20A and FIG. 20B. In some cases, genes can be separated by 2, 3, 4, 5, 6, 7, 8, 9, or up to 10 linkers.

A linker can be an engineered linker. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

TABLE 3
Linker polypeptide Sequences:

| SEQ ID No. | Sequence |
|---|---|
| 42 | APVKQ |
| 43 | GPVKQ |
| 44 | VPVKQ |
| 45 | IPVKQ |
| 46 | MPVKQ |
| 47 | APIKQ |
| 48 | GPIKQ |
| 49 | VPIKQ |
| 50 | IPIKQ |
| 51 | MPIKQ |
| 52 | APAKQ |
| 53 | GPAKQ |
| 54 | VPAKQ |
| 55 | IPAKQ |
| 56 | MPAKQ |
| 57 | APVRQ |
| 58 | GPVRQ |
| 59 | VPVRQ |
| 60 | IPVRQ |
| 61 | MPVRQ |
| 62 | APIRQ |
| 63 | GPIRQ |
| 64 | VPIRQ |
| 65 | IPIRQ |
| 66 | MPIRQ |
| 67 | APARQ |
| 68 | GPARQ |
| 69 | VPARQ |
| 70 | IPARQ |
| 71 | MPARQ |
| 72 | APVKN |
| 73 | GPVKN |
| 74 | VPVKN |
| 75 | IPVKN |
| 76 | MPVKN |
| 77 | APIKN |
| 78 | GPIKN |
| 79 | VPIKN |
| 80 | IPIKN |
| 81 | MPIKN |
| 82 | APAKN |
| 83 | GPAKN |
| 84 | VPAKN |
| 85 | IPAKN |
| 86 | MPAKN |
| 87 | APVRN |
| 88 | GPVRN |
| 89 | VPVRN |
| 90 | IPVRN |
| 91 | MPVRN |
| 92 | APIRN |
| 93 | GPIRN |
| 94 | VPIRN |
| 95 | IPIRN |
| 96 | MPIRN |
| 97 | APARN |
| 98 | GPARN |
| 99 | VPARN |
| 100 | IPARN |
| 101 | MPARN |

In some embodiments are polynucleotides encoding a polypeptide construct comprising a furin polypeptide and a 2A polypeptide, wherein the furin polypeptide and the 2A polypeptide are connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), proline (Pro)(P), phenylalanine (Phe)(F), methionine (Met)(M), tryptophan (Trp)(W). In some cases, the linker comprises a sequence provided in Table 3 disclosed herein. In some cases, a polypeptide linker may also include one or more GS linker sequences, for instance (GS)n (SEQ ID NO: 109), (SG)n (SEQ ID NO: 110), (GSG)n (SEQ ID NO: 111) and (SGS)n (SEQ ID NO: 112) wherein n can be any number from zero to fifteen.

Provided are methods of obtaining an improved expression of a polypeptide construct comprising: providing a polynucleotide encoding said polypeptide construct comprising a first functional polypeptide and a second functional polypeptide, wherein said first functional polypeptide and second functional polypeptide are connected by a linker polypeptide comprising a sequence with at least 60% identity to the sequence APVKQ (SEQ ID NO: 42); and expressing said polynucleotide in a host cell, wherein said expressing results in an improved expression of the polypeptide construct as compared to a corresponding polypeptide construct that does not have a linker polypeptide comprising a sequence with at least 60% identity to the sequence APVKQ (SEQ ID NO: 42). In some cases, the linker comprises a sequence provided in Table 3 disclosed herein. In some cases, a polypeptide linker may also include one or more GS linker sequences, for instance (GS)n (SEQ ID NO: 109), (SG)n (SEQ ID NO: 110), (GSG)n (SEQ ID NO: 111) and (SGS)n (SEQ ID NO: 112) wherein n can be any number from zero to fifteen. In some cases, the improved expression is at least about 10%, 15%, 20%, 25%, 30%, 35%, 40%, 45%, 50%, 55%, 60%, 65%, 70%, 75%, 80%, 85%, 90%, 95% or 100% higher as compared to a corresponding polypeptide construct that does not have a linker polypeptide comprising a sequence with at least 60% identity to the sequence APVKQ (SEQ ID NO: 42).

In preferred embodiments, a unique aspect of the present invention is provided and enabled by the specific sequential order of polypeptides encoded by constructs of the invention. Thus, in one aspect of the invention, specifically placing S100A1 protein first achieves and allows functional expression of biologically active S100A1 molecules because the remaining 2A tail on S100A1 was, surprisingly, discovered not to interfere with S100A1 biological activity. In contrast, it was discovered that placing S100A1 as the second gene resulted in incomplete cleavage of genes expressed via the construct.

Accordingly, analysis of pXoX expression using mass spectrometry, as indicated below in Table 13 showed that S100A1 was the only effector that did not exhibit furin cleavage resulting in removal of the C-terminal 2A peptide. Hence, there is no 2A C-terminal tail left on SDF1a or VEGF. Only S100A1 has a 2A linker-tail present. The exact ending amino acid sequence is PG. It cleaves between G and P of ending PGP sequence. Hence, the C-terminal linker tail on S100A1 as expressed from pXoX comprises the sequence (from N- to C-terminus) S101A1 polypeptide fused to (N-terminus)-RAKRAPVKQGSGATNFSLLKQAGDVEENPG-(C-terminus) (SEQ ID NO: 123) which may be referred to herein as the “2A tail”. For example, in one embodiment, from the initial methionine of S100A1 to the C-terminal end of the fp2a cleaved linker tail a first GOI of the invention comprises the sequence: MGSELETAMETLINVFHAHSGKEGDKYKLSKKELKELLQTELSGFLDAQKDVDAVDKVMK ELDENGDGEVDFQEYVVLVAALTVACNNFFWENSRAKRAPVKQGSGATNFSLLKQAGDV EENPG (SEQ ID NO: 124), which may be encoded by a polynucleotide sequence: ATGGGCAGCGAACTGGAAACCGCCATGGAGACTTTGATAAATGTTTTCCACGCGCATAG CGGCAAAGAAGGGGACAAGTACAAGCTGTCAAAAAAGGAGCTGAAAGAACTGCTGCA GACCGAATTGAGCGGCTTCCTGGACGCTCAGAAAGATGTCGATGCCGTCGACAAAGTG ATGAAAGAGCTTGACGAGAACGGTGACGGTGAAGTCGATTTTCAGGAATATGTGGTGC TGGTGGCCGCCCTTACTGTAGCATGCAACAATTTCTTTTGGGAAAATTCACGTGCAAAG CGTGCACCGGTGAAACAGGGAAGCGGAGCTACTAACTTCAGCCTGCTGAAGCAGGCTG GAGACGTGGAGGAGAACCCTGGA (SEQ ID NO: 125); or degenerate polynucleotides encoding the same amino acid sequence, or amino acid or nucleic acid sequences at least 45%, 50%, 55%, 60%, 65%, 70%, 75%, 80%, 90%, 95%, 97%, 98% or 99% identical to the above referenced 2A tail.

However, as provided by data presented herein, it has been demonstrated that this S100A1 tail is ‘inert’—i.e. does not affect S100A1 function (biological activity).

### Vector Modifications

A polynucleotide vector useful for the methods and compositions described herein can be a good manufacturing practices (GMP) compatible vector. For example, a GMP vector may be purer than a non-GMP vector. In some cases, purity can be measured by bioburden. For example, bioburden can be the presence or absence of aerobes, anaerobes, sporeformers, fungi, or combinations thereof in a vector composition. In some cases, a pure vector can be endotoxin low or endotoxin free. Purity can also be measured by double-stranded primer-walking sequencing. Plasmid identity can be a source of determining purity of a vector. A GMP vector of the invention can be from 10% to 99% more pure than a non-GMP vector. A GMP vector can be from 10%, 15%, 20%, 25%, 30%, 35%, 40%, 45%, 50%, 55%, 60%, 65%, 70%, 75%, 80%, 85%, 90%, 95%, 96%, 97%, 98%, or 99% more pure than a non-GMP vector as measured by the presence of bioburden, endotoxin, sequencing, or combinations thereof.

In some cases, a terminator sequence at the end of the first gene program is used. A terminator sequence can ensure that a transcript is terminating prior to initiating a second gene program. For example, an expression vectors may contain sequences necessary for the termination of transcription and for stabilizing an mRNA. Such sequences are commonly available from the 5′ and, occasionally 3′, untranslated regions of eukaryotic or viral DNAs or cDNAs. These regions can contain nucleotide segments transcribed as polyadenylated fragments in the untranslated portion of the mRNA. Cells comprising the expression vector are grown under conditions that provide for expression of the desired polypeptide, either in vivo or in vitro.

In some cases, a spacer sequence can be used at the end of a first polypeptide encoded by a polynucleotide in a vector. In other cases, a spacer sequence can be used at the end of a second gene in a vector. A spacer sequence can also be used following a first gene and a second gene in a vector.

These vectors can be used to express a polypeptide encoded by a gene, or portion of a gene of interest. A gene of portion or a gene can be inserted by using any method, viral or non-viral. For example; a method can be a non-viral based technique.

In some cases, vector modifications can be made. For example, a modification can include the addition of an inducible gene switch for controlled gene expression, changes in order and/or selection of cardiac effector genes to be combined, promoter replacement (e.g., strong, left ventricle specific expression), and combinations thereof.

In certain embodiments, translation initiation sequences preceding a cardiac effector polypeptide coding sequence in vectors of the invention comprise a Kozak consensus sequence. This may be indicated in descriptions provided herein as “Kozak”=“Yes”.

In certain embodiments, translation initiation sequences preceding a cardiac effector polypeptide coding sequence in vectors of the invention lack a Kozak consensus sequence. This may be indicated in descriptions provided herein as “Kozak”=“No”.

In certain embodiments, expression of any one, two, or three cardiac effector polypeptides (such as SDF1, S100A1 and VEGF191) may be driven via a constitutive or inducible promoter. In certain cases, expression of at least two genes is equal. In other cases, expression of at least two genes is not equal. In some cases, an upstream gene may be expressed at a higher concentration than a downstream gene. In other cases, a downstream gene may be expressed at a higher concentration than an upstream gene. A linker may be involved in controlling levels of expression. For example, a cleavable linker may be engineered such that cleavage is more efficient or less efficient.

In some cases, an inducible promoter can be a small molecule ligand-inducible two polypeptide ecdysone receptor-based gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/1J52002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. 20140308247) each of which is incorporated by reference in its entirety.

In some cases, a ligand used for dose-regulated control of ecdysone receptor-based inducible gene switch regulation can be selected from any of, but without limitation to, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

In some embodiments, two or three genes of interest (for example, but not limited to, cardiac effector genes) that express polypeptides of interest, can be incorporated into and/or expressed from a single vector under control of a single promoter. In certain embodiments the single promoter is a constitutive promoter. In certain embodiments the single promoter is a constitutive tissue-specific promoter. In certain embodiments the single promoter is a small molecule ligand-inducible ecdysone receptor-based promoter.

In certain embodiments, expression of the gene switch polypeptides is under control of a myosin light chain (MLC) promoter.

Vectors can be delivered in vivo by administration to a subject, typically by systemic administration (e.g., intravenous, intraperitoneal, intramuscular, subdermal, or intracranial infusion) or topical application, as described below. Alternatively, vectors can be delivered to cells ex vivo, such as cells explanted from a subject (e.g., cardiomyocytes, cardiac tissue biopsy), followed by reimplantation of the cells into a subject, usually after selection for cells which have incorporated the vector. Prior to or after selection, the cells can be expanded.

The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

The efficiency of integration of a gene into a cell (e.g., but not limited to, a cardiomyocyte), can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Certain aspects disclosed herein can utilize vectors. For example, vectors that can be used include, but not limited to, Bacterial: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWL-neo, pSv2cat, pOG44, pXT1, pSG (Stratagene) pSVK3, pBPv, pMSG, pSVL (Pharmiacia). Also, any other plasmids and vectors can be used as long as they are replicable and viable in a selected host. Any vector and those commercially available (and variants or derivatives thereof) can be engineered to include one or more recombination sites for use in the methods. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBacl 11, pCDM8, pcDNA1, pZeoSV, pcDNA3 pREP4, pCEP4, and pEBVHis (Invitrogen, Corp.), and variants or derivatives thereof. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSYSPORTI (Invitrogen) and variants or derivatives thereof. Additional vectors of interest can also include pTrxFus, pThioHis, pLEX, pTrcHis, pTrcHis2, pRSET, pBlueBa-cHis2, pcDNA3.1/His, pcDNA3.1(−)/Myc-His, pSecTag, pEBVHis, pPIC9K, pPIC3.5K, pA081S, pPICZ, pPICZA, pPICZB, pPICZC, pGAPZA, pGAPZB, pGAPZC, pBlue-Bac4.5, pBlueBacHis2, pMelBac, pSinRep5, pSinHis, pIND, pIND(SP1), pVgRXR, pcDNA2.1, pYES2, pZEr01.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; X ExCell, X gtl1, pTrc99A, pKK223-3, pGEX-1X T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-lb(+), pT7Blue®, pT7Blue-2, pCITE-4-abc(+), pOCUS-2, pTAg, pET-32L1C, pET-30LIC, pBAC-2 cp LIC, pBACgus-2 cp LIC, pT7Blue-2 LIC, pT7Blue-2, X SCREEN-1, X BlueSTAR, pET-3abcd, pET-7abc, pET9abcd, pET 11 abcd, pETI2abc, pET-14b, pET-15b, pET-16b, pET-17b-pET-17xb, pET-19b, pET-20b(+), pET-21abcd(+), pET-22b(+), pET-23abcd(+), pET-24abcd (+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFPN, pEGFP-C,

pEBFP, pGFPuv, pGFP, p6×His-GFP, pSEAP2-Basic, pSEAP2-Contral, pSEAP2-Promoter, pSEAP2-Enhancer, p I3 gal-Basic, pl3 gal-Control, p I3 gal-Promoter, p I3 gal-Enhancer, pCMV, pTet-Off, pTet-On, pTK-Hyg, pRetro-Off, pRetro-On, pIRESlneo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, 2Xgt10, Xgtl1, pWE15, and X TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phag-escript, pCAL-n-EK, pCAL-n, pCAL-c, pCAL-kc, pET-3abcd, pET-llabcd, pSPUTK, pESP-1, pCMVLacI, pOPRSVI/MCS, pOPI3 CAT, pXT1, pSG5, pPbac, pMbac, pMClneo, pMClneo Poly A, pOG44, p0G45, pFRTI3GAL, pNE0I3GAL, pRS403, pRS404, pRS405, pRS406, pRS413, pRS414, pRS415, and pRS416 from Stratagene, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp, and variants or derivatives thereof.

### Therapy Indications

Provided herein are methods and compositions for improving vasculogenesis in a subject. Also provided are methods and compositions for treatment for cardiovascular disease. In some cases, a cardiovascular disease can be cardiomyopathy. Cardiomyopathy can be defined by a pathologically abnormal myocardium. There can be four major classifications of cardiomyopathy: dilated (DCM), hypertrophic (HCM), restrictive (RCM), and arrhythmogenic RV (ARVC). Vectors comprising polynucleotides described herein can be used to treat cardiomyopathy using gene therapy. In some cases, at least one additional therapy is also administered before, during, after, or any combination thereof of a gene therapy treatment.

In some cases, a polypeptide can be incorporated in a polynucleotide sequence described herein and introduced to a subject with a disorder, or in need of improved vasculogenesis. For example, a subject can have a cardiomyopathy disorder and can be treated by administration of a polynucleotide described herein to treat the disorder. In some cases, a polynucleotide encoding a gene or a polypeptide described herein can be introduced by gene therapy. Genes and polypeptides that can be introduced to a subject with a cardiomyopathy can include for instance: ATP-binding cassette, sub-family C, member 9 (ABCC9), Actin, α, cardiac muscle 1 (ACTC1), Actinin, α2 (ACTN2), Ankyrin repeat domain 1 (cardiac muscle) (ANKRD1), BCL2-associated athanogene 3 (BAG3), Calsequestrin 2 (cardiac muscle) (CASQ2), Caveolin 3 (CAV3), COX15 homolog, cytochrome c oxidase assembly protein (COX15), Crystallin α B Cysteine and glycine-rich protein 3 (CRYAB), Cysteine and glycine-rich protein 3 (CSRP3), Cardiotrophin 1 (CTF1), Desmin (DES), Dystrophin (DMD), DnaJ (Hsp40) homolog, subfamily C, member 19 (DNAJC19), Desmocollin 2 (DSC2), Desmoglein 2 (DSG2), Desmoplakin (DSP), Dystrobrevin, α (DTNA), Emerin (EMD), Eyes absent homolog 4 (EYA4), Four and a half LIM domains 2 (FHL2), Fukutin (FKTN), Forkhead box D4 (FOXD4), Galactosidase, α (GLA), Junction plakoglobin (JUP), Laminin, a4 (LAMA4), Lysosomal-associated membrane protein 2 (LAMP2), LIM domain binding 3 (LDB3), Lamin A/C (LMNA), Myosin binding protein C, cardiac (MYBPC3), Myosin, heavy chain 6, cardiac muscle, α (MYH6), Myosin, heavy chain 7, cardiac muscle, α (MYH7), Myosin, light chain 2, regulatory, cardiac, slow (MYH12), Myosin, light chain 3, alkali; ventricular, skeletal, slow (MYL3), Myosin light chain kinase 2 (MYLK2), Myozenin 2 (MYOZ2), Nexilin (F actin binding protein) (NEXN), Plakophilin 2 (PKP2), Phospholamban (PLN), Protein kinase, AMP-activated, γ2, non-catalytic subunit (PRKAG2), Presenilin 1 (PSEN1), Presenilin 2 (PSEN2), RNA binding motif protein 20 (RBM20), Ryanodine receptor 2 (cardiac) (RYR2), Sodium channel, voltage-gated, type V, α subunit (SCN5A) Succinate dehydrogenase complex, subunit A, flavoprotein (SDHA), Sarcoglycan, δ (SGCD,) Spectrin repeat containing, nuclear envelope 1(SYNE 1), Spectrin repeat containing, nuclear envelope 2 (SYNE2), Tafazzin (TAZ), Titin-cap (telethonin) (TCAP), Transmembrane protein 43 (TMEM43), Thymopoietin (TMPO), Troponin C type 1 (slow) (TNNC1), Troponin I type 3 (cardiac) (TNNi3), Troponin T type 2 (cardiac) (TNNT2), Tropomyosin 1 (a)Titin (TPM1), TransthyreTIN (TTR), Titin (TTN), Vinculin (VCL), and any combination of portion thereof. Polypeptides or gene that can be introduced for treatment of a cardiovascular condition and/or improved vasculogenesis can be VEGF, SDF1, S100A1, or portions thereof or combinations thereof.

Provided herein are polynucleotide constructs comprising functional polypeptides attached by a linker construct disclosed herein. Also provided are methods for treatment, diagnosis and other therapeutic purposes comprising using or administering to a subject a polynucleotide described herein, or a polypeptide encoded thereby, or conjugates or derivatives thereof. The disclosed methods and compositions may be applied for treatment of diseases and conditions. An example of a condition can be cancer. A cancer or malignancy can include, but is not limited to: acute childhood lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's lymphoma, adult lymphocytic leukemia, adult non-Hodgkin's lymphoma, adult primary liver cancer, adult soft tissue sarcoma, aids-related lymphoma, aids-related malignancies, anal cancer, astrocytoma, bile duct cancer, bladder cancer, bone cancer, brain stem glioma, brain tumors, breast cancer, cancer of the renal pelvis and ureter, central nervous system (primary) lymphoma, central nervous system lymphoma, cerebellar astrocytoma, cerebral astrocytoma, cervical cancer, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood cerebral astrocytoma, childhood extracranial germ cell tumors, childhood Hodgkin's disease, childhood Hodgkin's lymphoma, childhood hypothalamic and visual pathway glioma, childhood lymphoblastic leukemia, childhood medulloblastoma, childhood non-Hodgkin's lymphoma, childhood pineal and supratentorial primitive neuroectodermal tumors, childhood primary liver cancer, childhood rhabdomyosarcoma, childhood soft tissue sarcoma, childhood visual pathway and hypothalamic glioma, chronic lymphocytic leukemia, chronic myelogenous leukemia, colon cancer, cutaneous t-cell lymphoma, endocrine pancreas islet cell carcinoma, endometrial cancer, ependymoma, epithelial cancer, esophageal cancer, Ewing's sarcoma and related tumors, exocrine pancreatic cancer, extracranial germ cell tumor, extragonadal germ cell tumor, extrahepatic bile duct cancer, eye cancer, female breast cancer, Gaucher's disease, gallbladder cancer, gastric cancer, gastrointestinal carcinoid tumor, gastrointestinal tumors, germ cell tumors, gestational trophoblastic tumor, hairy cell leukemia, head and neck cancer, hepatocellular cancer, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, kidney cancer, laryngeal cancer, lip and oral cavity cancer, liver cancer, lung cancer, lymphoproliferative disorders, macroglobulinemia, male breast cancer, malignant mesothelioma, malignant thymoma, medulloblastoma, melanoma, mesothelioma, metastatic occult primary squamous neck cancer, metastatic primary squamous neck cancer, metastatic squamous neck cancer, multiple myeloma, multiple myeloma/plasma cell neoplasm, myelodysplastic syndrome, myelogenous leukemia, myeloid leukemia, myeloproliferative disorders, nasal cavity and paranasal sinus cancer, nasopharyngeal cancer, neuroblastoma, non-Hodgkin's lymphoma, nonmelanoma skin cancer, non-small cell lung cancer, occult primary metastatic squamous neck cancer, oropharyngeal cancer, osteo-/malignant fibrous sarcoma, osteosarcoma/malignant fibrous histiocytoma, osteosarcoma/malignant fibrous histiocytoma of bone, ovarian epithelial cancer, ovarian germ cell tumor, ovarian low malignant potential tumor, pancreatic cancer, paraproteinemias, polycythemia vera, parathyroid cancer, penile cancer, pheochromocytoma, pituitary tumor, primary central nervous system lymphoma, primary liver cancer, prostate cancer, rectal cancer, renal cell cancer, renal pelvis and ureter cancer, retinoblastoma, rhabdomyosarcoma, salivary gland cancer, sarcoidosis sarcomas, sezary syndrome, skin cancer, small cell lung cancer, small intestine cancer, soft tissue sarcoma, squamous neck cancer, stomach cancer, supratentorial primitive neuroectodermal and pineal tumors, t-cell lymphoma, testicular cancer, thymoma, thyroid cancer, transitional cell cancer of the renal pelvis and ureter, transitional renal pelvis and ureter cancer, trophoblastic tumors, ureter and renal pelvis cell cancer, urethral cancer, uterine cancer, uterine sarcoma, vaginal cancer, visual pathway and hypothalamic glioma, vulvar cancer, Waldenstrom's macroglobulinemia, Wilms' tumor, and any other hyperproliferative disease, besides neoplasia, located in an organ system listed above.

Functional polypeptides connected by a linker polypeptide disclosed herein can be useful in methods and compositions described herein and may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred. Dysplasia can frequently be a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostatic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia. Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis. In some embodiments, a polypeptide construct encoded by a polynucleotide disclosed herein, comprising at least two functional polypeptides connected by a linker disclosed herein, is used to inhibit growth, progression, and/or metastasis of cancers, in particular those listed above. Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

In some embodiments, a polypeptide construct encoded by a polynucleotide disclosed herein, comprising at least two functional polypeptides connected by a linker disclosed herein, is used to treat diseases including, but are not limited to immune dysregulation disease and related autoimmune diseases, including Class III autoimmune diseases such as immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis obliterans, Sjögren's syndrome, primary biliary cirrhosis, Hashimoto's thyroiditis, thyrotoxicosis, scleroderma, chronic active hepatitis, rheumatoid arthritis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis, and also juvenile diabetes.

In some embodiments, a polypeptide construct encoded by a polynucleotide disclosed herein, comprising at least two functional polypeptides connected by a linker disclosed herein, is used to treat infectious diseases. Infectious diseases can be infection by pathogens such as bacteria, rickettsia, mycoplasma, protozoa, fungi, viruses, parasites, or other microbial agents. Examples include human immunodeficiency virus (HIV) causing AIDS, Mycobacterium of tuberculosis, Streptococcus agalactiae, methicillin-resistant Staphylococcus aureus, Legionella pneumophilia, Streptococcus pyogenes, Escherichia coli, Neisseria gonorrhoeae, Neisseria meningitides, Pneumococcus, Cryptococcus neoformans, Histoplasma capsulatum, Hemophilis influenzae B, Treponema pallidum, Lyme disease spirochetes, West Nile virus, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, sindbis virus, lymphocytic choriomeningitis virus, wart virus, blue tongue virus, Sendai virus, feline leukemia virus, reo virus, polio virus, simian virus 40, mouse mammary tumor virus, dengue virus, rubella virus, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

In some embodiments, a polypeptide construct encoded by a polynucleotide disclosed herein, comprising at least two functional polypeptides connected by a linker disclosed herein, is used in conjugation with a chemotherapeutic agent, or in addition to, or simultaneously with a chemotherapeutic agent. A “chemotherapeutic agent” or “chemotherapeutic compound” and their grammatical equivalents as used herein, can be a chemical compound useful in the treatment of cancer.

Genes and polypeptides encoded by polynucleotides useful for gene therapy can be introduced into a subject sequentially, concurrently, or a combination thereof. Genes and polypeptides to be administered to a subject can be on a single polynucleotide vector or on separate polynucleotide vectors. In some cases, a multigene vector is used to introduce multiple genes using a single vector construct.

### Polypeptide Constructs Encoded by Polynucleotides Described Herein:

Polynucleotide constructs described herein can be utilized to treat cardiovascular disease and/or improve vasculogenesis in a subject. For example, a molecular target for cardiovascular therapy can be VEGF-A, FGF4, Sarcoplasmic reticulum Ca 2+ ATPase, S100A1, beta-adrenergic receptor, Adenylyl-cyclase 6, or combinations thereof. In some cases, genes to be introduced for cardiovascular gene therapy can be SDF1, S100A1, VEGF191, or any combination thereof.

S100 is part of a family of Caβ+-modulated proteins implicated in intracellular regulatory activities. S100A1 can be the most abundant S100 protein isoform in a heart. It can promote cardiac contractile and relaxation function by enhancing activity of both ryanodine receptors (RYRs) and SERCA2a.

S100 can exert profound ionotropic actions through modulation of cardiomyocyte Ca2+ homeostasis and myofilament function independent of beta-adrenergic stimulation. S100A1 can interact in a Ca2+-dependent manner with the RyR and stabilizes the SERCA2a-PLN complex. S100A1 can also diminish the diastolic leak of Ca2+ and influences cardiac titin and mitochondrial F1-ATPase.

In some embodiments, an angiogenic polypeptide or gene can be utilized for cardiovascular therapy or for improved vasculogenesis as described herein. Administration of polynucleotides encoding angiogenic growth factors, such as VEGF-A165, angiopoietins, FGF, 126, HIF-1a26, or combinations thereof. These polypeptides can promote the development of collateral blood vessels in ischemia-related conditions, such as chronic critical limb ischemia, myocardial ischemia, angina, or peripheral arterial occlusive disease. Angiogenic factors can also induce formation of new vascular networks, which can make them suitable therapeutic options for treating acute coronary syndromes and peripheral vascular diseases. Several types of angiogenic factors can exhibit different properties have been explored in therapeutic applications for cardiovascular disease. For example, VEGF and subtypes of VEGF can be used. Subtypes of VEGF can comprise: VEGF-A, VEGF-B, VEGF-C, VEGF-D, placental growth factor (PlGF), and combinations thereof. Some of these factors also yield distinct isoforms. The intracellular signals of these VEGF subtypes can be mediated mainly by three different tyrosine kinase receptors: VEGFR1, VEGFR2, and VEGFR3. Specific interaction between these VEGF subtypes and their cognate cellular receptors can evoke a differential cellular response in endothelial cells and cardiomyocytes. In some cases, an isoform of VEGF-A, VEGF-A165, can have high angiogenic. VEGF-A165 can interact with VEGFR1 and VEGFR2. Interaction of VEGF-A165 with VEGFR1 on endothelial cells can contribute to vascular stability of newly formed vessels. Its interaction with VEGFR2 on endothelial cells can induce angiogenesis, vasculogenesis, and arteriogenesis, vasodilation, cell survival, and increase of cell permeability. Activation of VEGFR2 in newly formed cardiomyocytes can increase expression of anti-apoptotic proteins and reduced expression of pro-apoptotic proteins. In some cases, VEGFR2 activation can induce recruitment of local cardiac stem cells in ischemic areas.

A pro-angiogenic factor or gene can be expressed with a vector of the invention. For example, a pro-angiogenic factor, can be VEGF, basic fibroblast growth factor (bFGF), or transforming growth factor β-1(TGFβ-1), acidic fibroblast growth factor, angiogenin, hepatocyte growth factor, interleukin-8, placental growth factor, platelet-derived growth factor, pleiotropin, proliferin, tumor necrosis factor-alpha, to any combination thereof.

In some cases, cardiac effector genes or polypeptides used and incorporated into cardiac expression vectors of the invention can be selected from any one of as SDF1, S100A1 and VEGF191. In certain cases, a cardiac expression vector of the invention comprises or consists of any two genes selected from SDF1, S100A1 and VEGF191, in any combination of sequential order on the vector. In certain embodiments, a cardiac expression vector comprises a polynucleotide encoding SDF1, S100A1 and; VEGF191, in any combination of sequential order on the vector.

In certain embodiments, an SDF1 coding sequence comprises a polynucleotide encoding an SDF1 polypeptide. In certain embodiments, the SDF1 polypeptide is at least 45%, 50%, 55%, 60%, 65%, 70%, 75%, 80%, 85%, 90%, 95%, 97%, 98%, 99% or 100% identical to SEQ ID NO: 102. In certain cases, an SDF1 coding sequence comprises of an SDF1 codon optimized open reading frame (ORF), SEQ ID NO: 103.

In certain embodiments, a polynucleotide encoding SDF1 is at least 45%, 50%, 55%, 60%, 65%, 70%, 75%, 80%, 85%, 90%, 95%, 97%, 98%, 99% or 100% identical to SEQ ID NO: 103.

In certain cases, a S100A1 coding sequence comprises a polynucleotide encoding an S100A1 polypeptide. In certain embodiments, the S100A1 polypeptide is at least 45%, 50%, 55%, 60%, 65%, 70%, 75%, 80%, 85%, 90%, 95%, 97%, 98%, 99% or 100% identical to SEQ ID NO: 104. In certain embodiments, the S100A1 coding sequence comprises or consists of the S100A1 open reading frame (ORF). In certain cases, a polynucleotide encoding S100A1 is at least 45%, 50%, 55%, 60%, 65%, 70%, 75%, 80%, 85%, 90%, 95%, 97%, 98%, 99% or 100% identical to SEQ ID NO: 105.

In certain embodiments, a VEGF191 coding sequence can comprises a polynucleotide encoding a VEGF191 polypeptide. In certain embodiments, the VEGF191 polypeptide is at least 45%, 50%, 55%, 60%, 65%, 70%, 75%, 80%, 85%, 90%, 95%, 97%, 98%, 99% or 100% identical to SEQ ID NO: 106.

In certain cases, a VEGF191 coding sequence can comprises a VEGF191 open reading frame (ORF). In certain embodiments, the polynucleotide encoding VEGF191 is at least 45%, 50%, 55%, 60%, 65%, 70%, 75%, 80%, 85%, 90%, 95%, 97%, 98%, 99% or 100% identical to SEQ ID NO: 107.

TABLE 4
Polypeptide sequences and polynucleotide sequences

| SEQ ID | Gene Name | SEQUENCE (5′ TO 3′) |
|---|---|---|
| 102 | SDF-1 alpha | MNAKVVVVLVLVLTALCLSDGKPVSLSYRCPCRFFESHVA |
| | | RANVKHLKILNTPNCALQIVARLKNNNRQVCIDPKLKWIQ |
| | | EYLEKALNK |
| 103 | SDF-1 alpha | ATGAATGCCAAGGTCGTTGTGGTGCTTGTACTTGTGCTGA |
| | | CTGCTCTGTGTCTGAGCGACGGAAAACCAGTCTCCCTCAG |
| | | CTACAGGTGCCCATGCCGATTCTTCGAATCTCATGTGGCC |
| | | CGGGCCAATGTGAAGCACTTGAAAATCCTGAATACACCCA |
| | | ACTGCGCGTTGCAGATCGTGGCCCGCCTGAAAAATAATAA |
| | | TAGGCAGGTATGTATAGATCCAAAGCTTAAGTGGATCCAG |
| | | GAGTATCTGGAAAAGGCTCTCAATAAA |
| 104 | S100A1 | MGSELETAMETLINVFHAHSGKEGDKYKLSKKELKELLQT |
| | | ELSGFLDAQKDVDAVDKVMKELDENGDGEVDFQEYVVLVA |
| | | ALTVACNNFFWENS |
| 105 | S100A1 | ATGGGCAGCGAACTGGAAACCGCCATGGAGACTTTGATAA |
| | | ATGTTTTCCACGCGCATAGCGGCAAAGAAGGGGACAAGTA |
| | | CAAGCTGTCAAAAAAGGAGCTGAAAGAACTGCTGCAGACC |
| | | GAATTGAGCGGCTTCCTGGACGCTCAGAAAGATGTCGATG |
| | | CCGTCGACAAAGTGATGAAAGAGCTTGACGAGAACGGTGA |
| | | CGGTGAAGTCGATTTTCAGGAATATGTGGTGCTGGTGGCC |
| | | GCCCTTACTGTAGCATGCAACAATTTCTTTTGGGAAAATT |
| | | CA |
| 106 | VEGF191 | MNFLLSWVHWSLALLLYLHHAKWSQAAPMAEGGGQNHHEV |
| | (amino acids | VKFMDVYQRSYCHPIETLVDIFQEYPDEIEYIFKPSCVPL |
| | 27-191 of | MRCGGCCNDEGLECVPTEESNITMQIMRIKPHQGQHIGEM |
| | this sequence | SFLQHNKCECRPKKDRARQENPCGPCSERRKHLFVQDPQT |
| | form | CKCSCKNTDSRCKARQLELNERTCRCDKPRR |
| | VEGF165) | |
| 107 | VEGF191 | ATGAATTTTCTGCTCTCTTGGGTGCACTGGTCACTGGCAC |
| | | TGCTGCTGTATCTGCACCATGCAAAATGGTCCCAAGCAGC |
| | | TCCCATGGCAGAGGGAGGTGGACAGAATCATCATGAGGTT |
| | | GTCAAATTTATGGATGTCTACCAGCGGAGCTACTGCCACC |
| | | CAATTGAGACGTTGGTAGACATTTTTCAGGAATATCCAGA |
| | | CGAGATTGAGTACATTTTCAAGCCTAGCTGTGTGCCCTTG |
| | | ATGCGATGCGGTGGCTGTTGCAATGATGAGGGACTCGAGT |
| | | GTGTCCCCACCGAGGAAAGCAATATAACCATGCAAATCAT |
| | | GCGAATCAAACCCCACCAGGGCCAGCATATCGGCGAGATG |
| | | TCTTTCTTGCAACATAACAAATGCGAGTGTCGGCCAAAGA |
| | | AGGACAGGGCTCGCCAGGAAAATCCCTGTGGTCCTTGTTC |
| | | AGAGCGCAGGAAGCATCTTTTCGTCCAGGATCCGCAGACT |
| | | TGTAAATGTTCATGCAAGAATACCGATTCTAGGTGTAAGG |
| | | CGAGGCAACTCGAGCTTAACGAGAGAACCTGTAGGTGTGA |
| | | CAAACCTAGAAGA |
| 126 | VEGF 165 | APMAEGGGQNHHEVVKFMDVYQRSYCHPIETLVDIFQEYP |
| | (predicted | DEIEYIFKPSCVPLMRCGGCCNDEGLECVPTEESNITMQI |
| | mature form; | MRIKPHQGQHIGEMSFLQHNKCECRPKKDRARQENPCGPC |
| | i.e., | SERRKHLFVQDPQTCKCSCKNTDSRCKARQLELNERTCRC |
| | VEGF191 | DKPRR |
| | minus signal | |
| | peptide) | |
| 127 | VEGF165 | GCTCCCATGGCAGAGGGAGGTGGACAGAATCATCATGAGG |
| | (encoding | TTGTCAAATTTATGGATGTCTACCAGCGGAGCTACTGCCA |
| | predicted | CCCAATTGAGACGTTGGTAGACATTTTTCAGGAATATCCA |
| | mature form; | GACGAGATTGAGTACATTTTCAAGCCTAGCTGTGTGCCCT |
| | i.e., | TGATGCGATGCGGTGGCTGTTGCAATGATGAGGGACTCGA |
| | VEGF 191 | GTGTGTCCCCACCGAGGAAAGCAATATAACCATGCAAATC |
| | minus signal | ATGCGAATCAAACCCCACCAGGGCCAGCATATCGGCGAGA |
| | peptide) | TGTCTTTCTTGCAACATAACAAATGCGAGTGTCGGCCAAA |
| | | GAAGGACAGGGCTCGCCAGGAAAATCCCTGTGGTCCTTGT |
| | | TCAGAGCGCAGGAAGCATCTTTTCGTCCAGGATCCGCAGA |
| | | CTTGTAAATGTTCATGCAAGAATACCGATTCTAGGTGTAA |
| | | GGCGAGGCAACTCGAGCTTAACGAGAGAACCTGTAGGTGT |
| | | GACAAACCTAGAAGA |

### Pharmaceutical Compositions and Formulations:

The compositions described throughout can be formulation into a pharmaceutical medicament and be used to treat a human or mammal, in need thereof, diagnosed with a disease, e.g., cardiovascular disease. In some embodiments, compositions described herein utilized for cardiovascular therapy or for improved vasculogenesis as described herein. These medicaments can be co-administered with one or more vectors.

Vectors comprising polynucleotides described herein, including viral and non-viral vectors containing nucleic acids encoding engineered CRISPR, TALEN, Argonaut, transposon-based, ZEN, meganuclease, or Mega-TAL molecules, transposon and/or transgenes can also be administered directly to an organism for transfection or transduction of cells in vivo. Alternatively, naked DNA or mRNA comprising polynucleotides described herein can be administered. Administration is by any of the routes normally used for introducing a molecule into ultimate contact with blood or tissue cells including, but not limited to, injection, infusion, topical application and electroporation. More than one route can be used to administer a particular composition. Pharmaceutically acceptable carriers are determined in part by the particular composition being administered, as well as by the particular method used to administer the composition.

In some cases, vectors comprising polynucleotides described herein can be administered to a subject in need thereof in conjunction with, or separately with secondary therapies. A secondary therapy can be an agent for improved vasculogenesis, pulmonary hypertension, aldosterone receptor antagonists, angiotensin converting enzyme inhibitors, angiotensin receptor blockers, angiotensin receptor blockers and neprilysin inhibitors, antiadrenergic agents, centrally acting, antiadrenergic agents, peripherally acting antianginal agents, antiarrhythmic agents, group I antiarrhythmic agents, group II antiarrhythmics, group III antiarrhythmics, group IV antiarrhythmics, group V antiarrhythmics, anticholinergic chronotropic agents, antihypertensive combinations, ACE inhibitors with calcium channel blocking agents, ACE inhibitors with thiazides, angiotensin II inhibitors with calcium channel blockers, angiotensin II inhibitors with thiazides, antiadrenergic agents (central) with thiazides, antiadrenergic agents (peripheral) with thiazides, beta blockers with thiazides, potassium sparing diuretics with thiazides, beta-adrenergic blocking agents, cardio selective beta blockers, non-cardioselective beta blockers, calcium channel blocking agents, catecholamines, diuretics, carbonic anhydrase inhibitors, loop diuretics, potassium-sparing diuretics, thiazide diuretics, inotropic agents, peripheral vasodilators, renin inhibitors, sclerosing agents, vasodilators, vasopressin antagonists, vasopressors

Anti-angiogenic agents can also be used in conjunction with polynucleotides described herein. Suitable anti-angiogenic agents for use in the disclosed methods and compositions include anti-VEGF antibodies, including humanized and chimeric antibodies, anti-VEGF aptamers and antisense oligonucleotides. In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg.

Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

### Methods and Indications

Vectors comprising polynucleotides described herein can be constructed and introduced into a subject as described herein. These vectors can be used for gene therapy. These vectors can be used to treat disease in a recipient (e.g., a human). For example, polynucleotides described herein and/or polypeptides encoded by polynucleotides described herein can be administered to a subject to treat cardiovascular disease or improve vasculogenesis in the subject. A subject treated by a method described herein, or by contact with or administration of a composition described herein can be a mammalian subject who can be a human subject, a non-human primate, a canine mammal, a felid mammal or any other mammal.

Described herein is a method of treating a disease (e.g., cardiovascular disease) in a recipient comprising introducing at least one vector comprising at least one polynucleotides described herein into a subject with cardiovascular disease. In some cases, a vector is introduced to a cell.

In some cases, a cardiovascular disease can be cardiomyopathy. Cardiomyopathy can be defined by a pathologically abnormal myocardium. There can be four major classifications of cardiomyopathy: dilated (DCM), hypertrophic (HCM), restrictive (RCM), and arrhythmogenic RV (ARVC). Cardiomyopathy can be diagnosed through in vivo imaging, with either echocardiography or, increasingly, cardiac MRI. DCM can refer to enlargement of the heart, which often affects all four chambers, especially late in the disease. Most commonly, DCM can be associated with reduced LV function or systolic function, although early in the disease the LV may be dilated, with only minimally reduced function. In contrast, HCM can be characterized by increased LV wall thickness, often targeting the septum that separates the LV from the RV. RCM can be elusive in some cases, in part because the heart may appear morphologically close to normal, with only minor increased wall thickness or modestly decreased LV ejection fraction. The infiltrative process underlying RCM may not be readily detectable in vivo with even the most sensitive imaging technique. RCM can be characterized by impaired filling of the heart, known as diastolic dysfunction, which reduces cardiac output. ARVC can be characterized by reduced function and thinning of the RV with a fibrofatty infiltration that can be seen on MRI. In some cases, a cardiomyopathy patient can be treated with a vector of the invention.

A method provided herein can be used for treating or preventing disease including, but not limited to, cancer, cardiovascular diseases, lung diseases, liver diseases, skin diseases, or neurological diseases. A method provided herein can be used for improved vasculogenesis in a subject.

“Improving” and its grammatical equivalents as used herein can mean any improvement recognized by one of skill in the art. For example, improving cardiovascular disease can mean lessening arrhythmia, which can encompass a decrease, lessening, or diminishing of an undesirable effect or symptom. In another example, improving disease can mean lessening tumor load, which can encompass a decrease, lessening, or diminishing of an undesirable effect or symptom. For example, a subject may experience reduction of a tumor load, extended survival, complete remission, stabilization to name a few improvements.

Another indication of improvement can be the days a subject does not require therapy. For example, after treatment administered provided herein, a recipient can require no therapy for at least or at least about 1, 2, 3, 4, 5, 6, 7, 8, 9, 10 or more days. This can indicate that a treatment was successful. This can also indicate that there is no toxicity associated with the administration of the polynucleic acids described herein.

A subject can require no therapy for at least 1 day. A recipient can also require no therapy for at least 7 days. A recipient can require no therapy for at least 14 days. A recipient can require no therapy for at least 21 days. A recipient can require no therapy for at least 28 days. A recipient can require no therapy for at least 60 days. Furthermore, a recipient can require no therapy for at least 1, 2, 3, 4, 5, 6, 7, 8, 9, 10, or more years.

Another indication of improvement can be the days a recipient requires reduced therapy. For example, after the treatment provided herein, a recipient can require reduced therapy administrations for at least 1, 2, 3, 4, 5, 6, 7, 8, 9, 10 or more days. This can indicate that treatment was successful.

For example, a recipient can require reduced therapy for at least 1 day. A recipient can also require reduced therapy for at least 7 days. A recipient can require reduced therapy for at least 14 days. A recipient can require reduced therapy for at least 21 days. A recipient can require reduced therapy for at least 28 days. A recipient can require reduced therapy for at least 60 days.

Furthermore, a recipient can require reduced therapy for at least 1, 2, 3, 4, 5, 6, 7, 8, 9, 10 or more years.

In treatment of cardiac diseased, percutaneous coronary artery catheterization can be a minimally invasive procedure that can allow for homogeneous gene delivery to each region of a heart. In some cases, gene delivery can be impeded in patients with severe coronary artery disease. For example, during coronary artery infusion, a vector can be injected in a catheter without interruption of coronary flow.

In some cases, enhanced vector residence time in coronary circulation can be achievable with coronary venous blockade. For example, antegrade coronary infusion with a short occlusion of both a coronary artery and a coronary vein enhanced myocardial gene expression can enhance vector residence time.

In other cases, to maximize duration of a vector exposure to an endothelium while minimizing systemic distribution, a cardiac recirculation approach can be used. In this case, an extracorporeal device can drain blood from a coronary sinus using an occlusion catheter and return oxygenated coronary venous blood to a left main coronary artery via a peristaltic pump (V-Focus; Osprey Medical Inc.) Utilizing this method can allow for selective administration of endothelial permeabilizing agents without systemic side effects.

In some cases, transfection efficacy can correlate with coronary flow as well as exposure time and vector concentration. For example, antegrade coronary artery infusion supported by an increased coronary flow, for example using an intra-aortic balloon pump, might further enhance cardiac gene transfer

In some cases, a direct injection of a vector into the heart can be performed. A vector can be injected epicardially or endocardially into a target area. In some cases, a direct injection can bypass an endothelial barrier. This can result in a high local concentration at an injection site. In some cases, direct injection can avoid exposure to circulating blood. In other cases, a direct injection of a vector into a heart can avoid deactivation of a vector by circulating DNases or neutralizing antibodies. A direct vector injection can also reduce exposure of a vector to off-target organs, although local administration cannot completely avoid some systemic vector distribution. Low volumes at high vector concentrations may also increase vector retention in a myocardium.

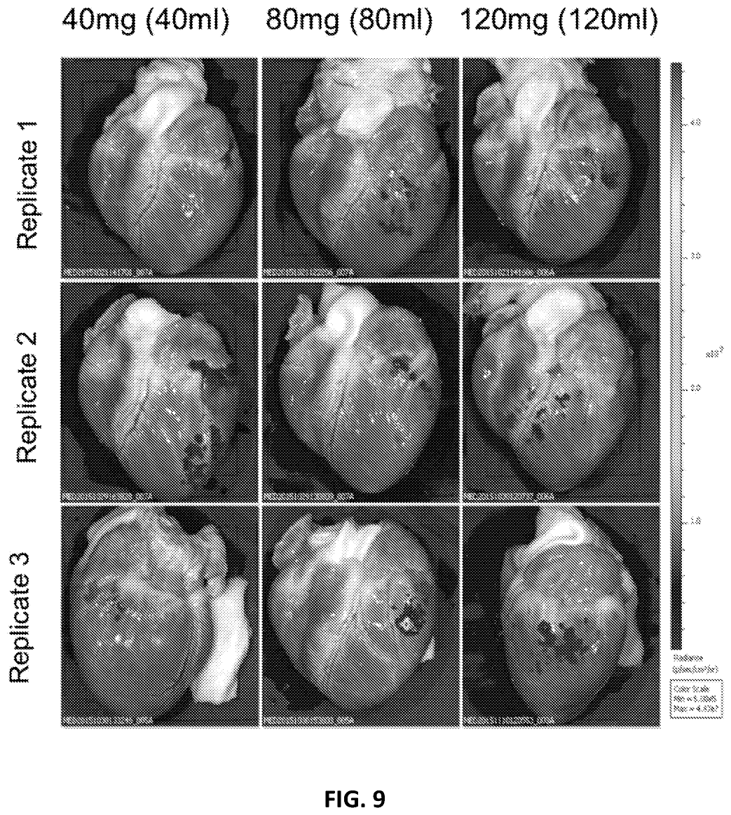

In some cases, a vector can be introduced during a thoracotomy. A vector can also be introduced pericardially. In some cases, a vector can be introduced using retrograde perfusion, for instance as disclosed in FIG. 9.

After gene therapy, the transfected cells can be functional in the recipient. Functionality can in some cases determine whether gene therapy was successful. For example, the transfected cells can be functional for at least or at least about 1, 2, 3, 4, 5, 6, 7, 8, 9, 10 or more days. This can indicate that gene therapy was successful. This can also indicate that there is no rejection of the transplanted cells or vectors.

In certain instances, transfected cells can be functional for at least 1 day. Transfected cells can also functional for at least 7 days. Transfected cells can be functional for at least 14 days. Transfected cells can be functional for at least 21 days. Transfected cells can be functional for at least 28 days. Transfected cells can be functional for at least 60 days.

### Routes of Administration

Previously, delivery techniques of therapeutics into the heart have included intramyocardial administration (transepicardially or transendocardially) or antegrade delivery into the coronary artery. The transepicardial route suffers the drawback of surgical invasiveness. The transendocardial approach requires complex electromagnetic mapping using systems such as the NOGA device which is not applicable for patients with thinned myocardium, as this technique may cause perforation. Other devices such the Biocardia—Helical infusion catheter or the Celyad—C-Cath also have the limitations of wall thickness.

Antegrade administration into the coronary artery is associated with a lower biologic retention [12], as compared to intramyocardial administration. Additionally, administration of biologics into the coronary artery has been shown to increase risk of coronary embolism and ST segment elevations [13] [14] [15] [16] [17]. Also with pDNA administration, the direct contact with a large blood volume may decrease the effect before entering into the target tissue.

### Retrograde Administration

In humans, the coronary sinus drains the venous system of the ventricular cavities, which are responsible for the majority of cardiac contraction. The technique of retrograde delivery into the coronary sinus has been widely used for the administration of cardioplegia solution due in part to superior distribution of the solution throughout the myocardium as compared to antegrade delivery [18]. Additionally, the procedure has been demonstrated clinically safe for administration of oxygenated blood during high risk percutaneous transluminal coronary angioplasty [19] [20] [21]. The process of retrograde administration into the coronary sinus involves temporary occlusion of afferent coronary circulation by means of a balloon catheter followed by administration against the outflowing blood. This results in the solution entering the myocardium via post capillary venules. In contrast to arterioles or capillaries, post-capillary venules have the smallest vessel diameter and conceptually would allow for greatest transfer of material into the interstitium [22]. Physiologically, it is known that post-capillary venules are a major target of immune/inflammatory cell migration across the endothelium, in part due to expression of adhesion molecules such as ELAM-1 [23], ICAM-1 [24], CD18 [25], and CD44 [26], and in part because of biomechanical properties. Patel et al demonstrated in the REVIVE Trial, using the same delivery catheter as proposed in this pre-clinical study (Cook Regentec), that over 3 billion nucleated cells can safely be delivered in a volume of 60 cc into the coronary sinus of patients with end-stage CHF [27].

### Gene Therapy by Retrograde Administration

Several studies have successfully utilized retrograde administration in the area of cardiac regeneration. Boekstegers et al [32], delivered adenovirus expressing beta-gal and Luciferase into the porcine myocardium comparing antegrade delivery into the coronary artery or retrograde via the anterior cardiac vein. Significantly elevated expression of the gene in infarct tissue in a homogenous manner was observed via the retrograde method as compared to antegrade. Similar results were reported by Alino et al [33], who observed interstitial expression of eGFP in porcine hearts that were injected in the retrograde manner with naked DNA. In another study, administration of beta-gal encoding plasmid using the retrograde method in pigs resulted in higher myocardial gene expression in comparison to antegrade and intramuscular administration [34]. This superior level of gene expression in comparison to intramyocardial delivery was reproduced in other studies [35]. The use of retrograde administration has also been performed successfully for delivery of protein therapeutics. Von Degenfeld et al [36], reported a porcine study in which retrograde administration of FGF-2 protein was used to prevent experimentally-induced stenosis. Levels of radiolabelled FGF-2 in the myocardium of pigs treated with retrograde were almost twice the levels achieved using antegrade infusion. Additionally, significant improvements in transmural blood flow and regional myocardial function were reported when FGF-2 was administered via the retrograde method. The safe use retrograde delivery of SDF-1α pDNA in heart failure patients was also reported (RETRO-HF).

Accordingly, a preferred embodiment of the present invention comprises use of retrograde administration via the coronary sinus. Another embodiment of the present invention comprises use of intracoronary or intramyocardial routes of administration.

### Ultrasound Targeted Microbubble Destruction Gene Delivery (UTMD)

Gas-filled microbubbles are useful ultrasound contrast agents. In some embodiments, microbubbles are used for delivering vectors, genes and/or constructs described herein to tissues and/or organs in a subject.[65] In some cases, the delivery can be organ or tissue-specific. When sonified with ultrasound near their resonance frequency, microbubbles oscillate. With higher ultrasound energies, oscillation amplitudes increase, leading to microbubble destruction. This phenomenon is used to deliver a vector or composition described herein into a target organ, for instance, by administering microbubbles loaded with compositions or gene therapy vectors described herein, for instance by i.v. injection, and subsequently exposing to ultrasound energy resulting in microbubble destruction at a target site.

In an embodiment of UTMD, bioactive molecules, such as plasmid vectors described herein, polynucleotides encoding gene constructs described herein, are added to, for instance, the cationic shells of lipid microbubble contrast agents. These vector-carrying microbubbles can be administered to the subject for instance, intravenously or directly to the left ventricle of the heart amongst other administration options. In some subjects, the microbubbles they can be infused through an intracoronary catheter. The subsequent delivery from the circulation to a target organ occurs by acoustic cavitation at a resonant frequency of the microbubbles. In some embodiments, the mechanical energy generated by the microbubble destruction may result in transient pore formation in or between the endothelial cells of the microvasculature of the targeted region. In some embodiments, the transfection efficiency into and across the endothelial cells is enhanced, and transgene-encoding vectors are deposited into the surrounding tissue. As such, in some cases, wherein a vector described herein is delivered by UTMD, any additional plasmid DNA remaining in the circulation can be degraded by nucleases in the blood, thereby resulting in highly specific target-organ transfection to the organs exposed to ultrasound sonication, and reducing the likelihood of delivery to non-sonicated tissues.

### EXAMPLES

### Example 1: Transfection of Cardiomyocytes for Att Site Mediated Recombination

Cardiomyocytes were transfected with luciferase vectors with or without (i.e., plus (+) or minus (−)) att-site, and plus (+) or minus (−) SpBC2 recombinase. Luciferase expression was then monitored over time to assess att-site mediated recombination. To determine site specificity and efficacy of SpBC2 and its proposed site of activity (Att-site), cardiomyocytes were co-transfected with a SpBC2 plasmid and a Firefly luciferase (“FLUC” or “Luc”) plasmid vector containing att-site around the gene of interest. Controls included were Att-Luc plasmid without SpBC2 (to measure random integration), No-Att-Luc plasmid with SpBC2 (to measure non-att-site specific integration), and No-Att-Luc plasmid without SpBC2 (to measure transient expression). Cells were transfected via DNAfectin (Applied Biological Materials Inc., Richmond, BC, Canada) and cultured over 14 days, periodically sub-culturing cells, testing cells by luciferase assay, and harvesting cells for qPCR assays.

Transfection was performed by utilizing complete growth media and transfection media (Prigrow I without FBS/antibiotics). At least 1 hour prior to transfection, media was removed from cardiomyocytes and replaced with fresh growth media. 3 mL/well in 6 well plate or 1.5 mL/well in 12 well plates. The DNAfectin reagent was warmed to room temperature prior to use and vortexed briefly to mix. The various combinations of DNAfectin-plasmid DNA complexes were prepared in transfection media as follows:

| Plasmid Vector | DNA |
|---|---|
| Combination | (μg) |
| Att-Luc | 0.375 |
| SPBc2 | 0.375 |
| Att-Luc | 0.375 |
| Stuffer | 0.375 |
| Luc | 0.375 |
| SPBc2 | 0.375 |
| Luc | 0.375 |
| Stuffer | 0.375 |

“Att-Luc” = vector with att site and luciferase gene; “Luc” = vector with luciferase gene, no att site; “SPBc2” = vector encoding SPBc2 recombinase; and, “Stuffer” = (or pStuffer) is a plasmid with the same backbone configuration as pXoX, with the open reading frame (ORF) replaced with a non-expressing, similar-sized non-effector sequence.

DNA was added to the transfection media. The mixture was incubated at room temperature for 20 minutes. 300 μL per well DNAfectin-DNA complex was added to cells.

Passage and Harvest for qPCR, Luciferase Assay

48 hours post-transfection, media was removed from the cells in the 12 well plates. Cells were trypsinized and harvested into microfuge tubes. Cells were then resuspended in 1 mL fresh growth media. Cells were seeded into a fresh 12 well plate at 5e4 cells/well and luciferase was measured. The remaining cells were harvested for qPCR.

### Results:

Over a two week course, FLUC expression from the Att-Luc+SPBc2 recombinase decreased roughly 10-fold, while FLUC expression from the other transfection conditions (either without att-site or without recombinase) decreased by 100- to 1000-fold, returning nearly to background levels of expression (data not shown). Att-site mediated recombination of the FLUC GOI (Genes of Interest) into the cardiomyocyte genome was also observed (data not shown).

### Example 2: HUVEC Proliferation Assay

A Human Umbilical Vein Endothelial Cell (HUVEC) proliferation assay was used to assess the functionality of VEGF165 expressed by pXoX. Serum-starved HUVECs were treated with conditioned media from patients (dilated, hypertrophic), or healthy iPSC-derived cardiomyocytes transfected with pXoX or a single effector control (pVEGF165 positive control, pSDF-1α negative control). After 96 hours of incubation, HUVEC proliferation was quantitated using a luminescence assay. Assay were repeated three times (e.g., CM-1, CM-2, CM-3).

### Induced Pluripotent Stem Cells

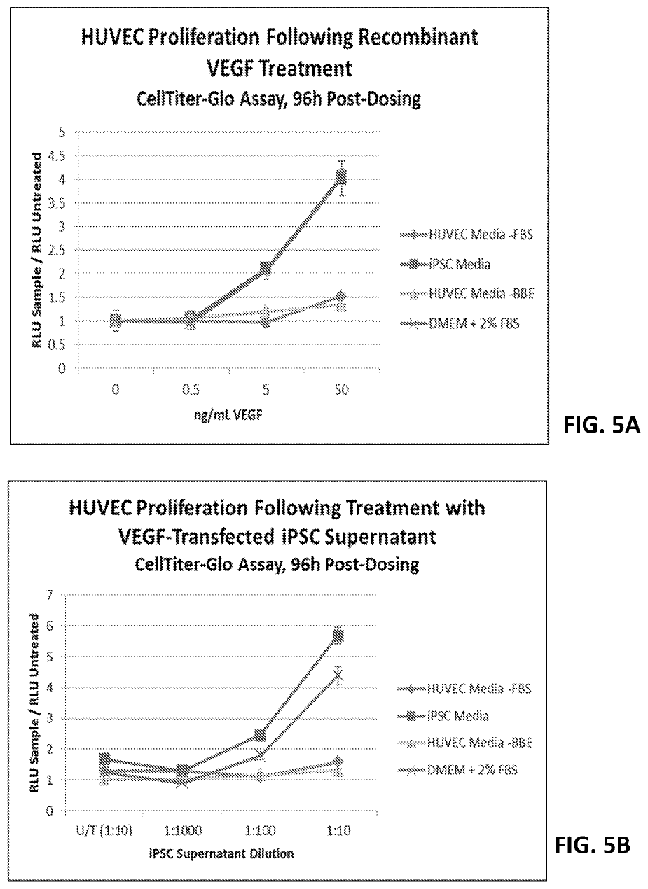

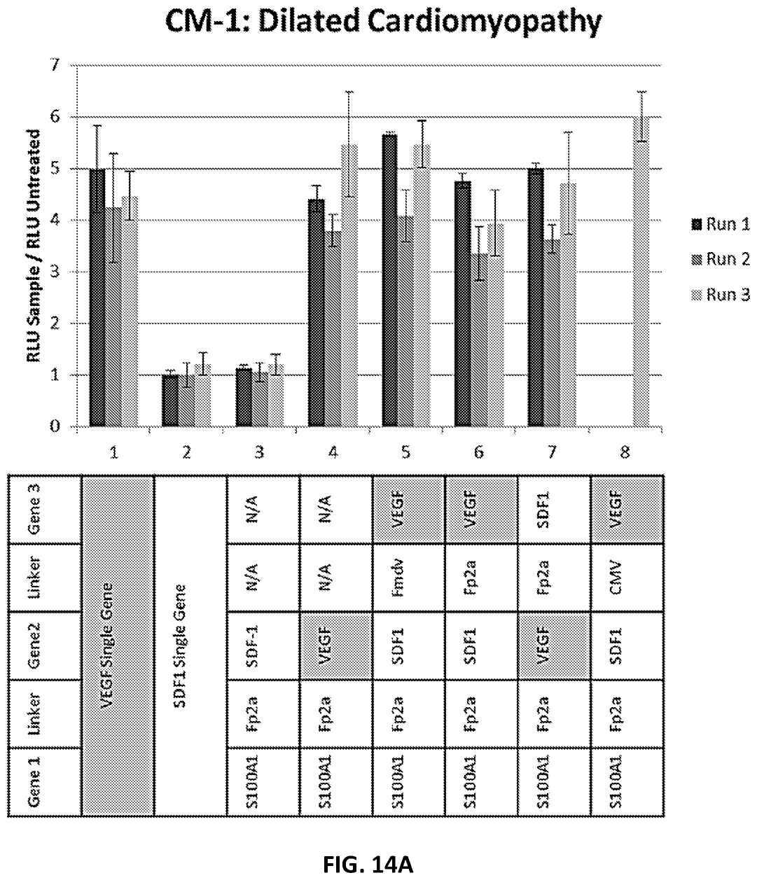

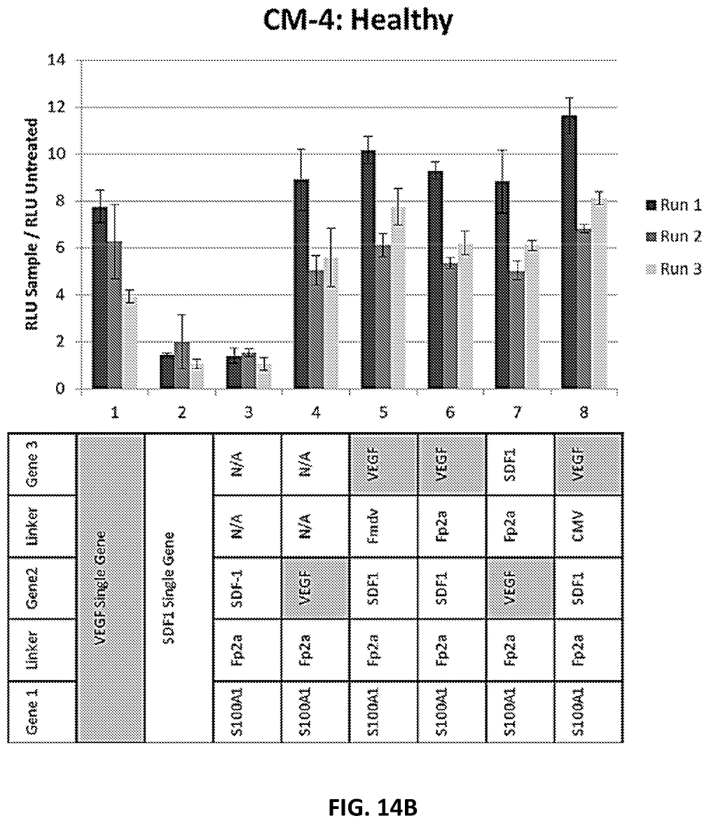

iPSC cardiomyocyte cells were transfected with singe gene, dual-gene, or triple-gene vectors encoding for S100A1 and VEGF; S100A1, SDF1, and VEGF; or VEGF alone. 48 hours post transfection cellular supernatant was collected. HUVECs were seeded on 6-well plates at a density of approximately 1:10, 1:100, and 1:1000 in the supernatant collected from the transfected iPSC cells. 96 hours following introduction of the supernatant, HUVEC proliferation was measured by CellTiter-Glo Assay, FIG. 5A and FIG. 5B show experimental data. FIG. 14A and FIG. 14B show similar results for iPSC cells derived from a dilated cardiomyopathy patient.

Cardiomyocytes from Cardiomyopathy Patients and Healthy Controls

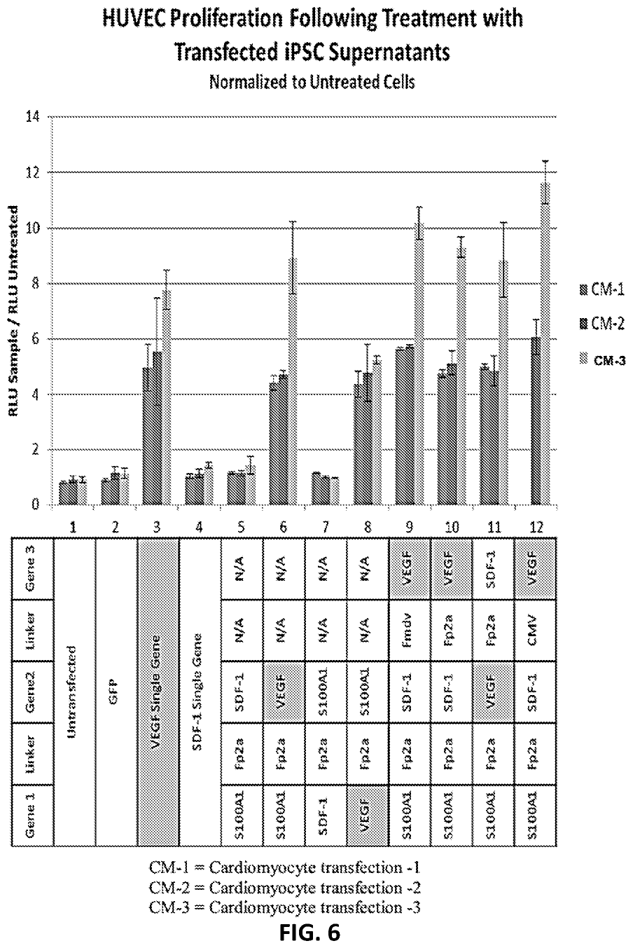

Cardiomyocyte-iPSC cells were obtained from patients with dilated cardiomyopathy (DCM), hypertrophy cardiomyopathy (HCM) or healthy controls. iPSC cells were transfected with singe gene, dual-gene, or triple-gene vectors encoding for S100A1 and VEGF; S100A1, SDF1, and VEGF; or VEGF alone. 48 hours post transfection cellular supernatant was collected. HUVECs were seeded on 6-well plates at a density of approximately 1:10, 1:100, and 1:1000 in the supernatant collected from the transfected iPSC cells. 96 hours following introduction of the supernatant, HUVEC proliferation was measured by CellTiter-Glo Assay, FIG. 6 shows experimental data.

### Results

iPSC-CMs transfected with singe gene, dual-gene, or triple-gene vector constructs showed 4-10-fold increase in HUVEC proliferation in both cases. Thus, results successfully demonstrate that VEGF is functional (i.e., biologically active) when expressed as part of multi-genic linked constructs from dual-gene and triple-gene expression vectors when using linkers provided herein (i.e., induce HUVEC proliferation similar to that observed with a single VEGF165 effector plasmid).

### Example 3: Endothelial Tube Formation Assay

The Endothelial Tube Formation Assay (CBA200, Cell Biolabs Inc., San Diego, CA, USA) will be used in addition to the HUVEC proliferation assay. ECM gel will be thawed at 4° C. and mixed to homogeneity using cooled pipette tips. Cell culture plates (96-well) were bottom-coated with a thin layer of ECM gel (50 μl/well), which will be left to polymerize at 37° C. for 60 min. HUVEC (2-3×10⁴ cells) will be seeded into the cell culture plate in supernatant collected from iPSC cardiomyocyte cells that were transfected with singe gene, dual-gene, or triple-gene vectors encoding for S100A1 and VEGF connected by a linker described herein; S100A1, SDF1, and VEGF connected by a linker described herein; or VEGF alone. 150 μl of the supernatant-HUVEC mixture will be added to each well on the solidified ECM gel. The plates will be incubated at 37° C. for 12-18 hr. and the endothelial tubes will be quantified using a fluorescent microscope after staining with Calcein AM. Three microscope fields will be selected at random and photographed. Tube forming ability will be quantified by counting the total number of cell clusters (knots) and branches under a 4× objective and four different fields per well. The results will be expressed as mean fold change of branching compared with the control groups. Each experiment will performed at least three times.

### Example 4: SDF-1α Function—Migration Assay

Chemotaxis experiments were performed using a 24-well transwell chemotaxis chamber technique (Millipore, Billerica, MA, USA). Briefly, Peripheral blood leukocytes (PBLs) were isolated from samples obtained from healthy volunteers and transfected with double gene or triple-gene vectors encoding for S100A1 and VEGF connected by a linker described herein, or S100A1, SDF1, and VEGF connected by a linker described herein. A total of 1×10⁵ PBLs in 200 μL medium were seeded into the upper chamber (pore size, 8 m). For the inhibition experiment, half of the PBLs were pre-incubated with 10 nmol/L CXCR4 antagonist (AMD3100) for 30 min prior to seeding. Then, PBLs and medium were transferred into the upper chamber. The chamber was then incubated for 12 h at 37° C. in a humidified atmosphere with 5% CO₂. The membrane (Millipore) was removed and its upper surface was wiped away with a cotton swab to remove the immobile PBLs. The membrane was then fixed in neutral formalin for 10 min at room temperature and then stained with 0.10% crystal violet for 5 min. The number of PBLs that have migrated to the lower surface of the membrane was counted in 10 random high-power fields (HPFs) under a light microscope (Nikon Eclipse, Nikon Instruments, Inc., Melville, NY, USA).

A chemotactic index (CI) was calculated to express stimulated migration: CI=stimulated migration (number of CSCs per HPF)/random migration (number of CSCs per HPF). Each assay was performed in triplicate wells.

### Results

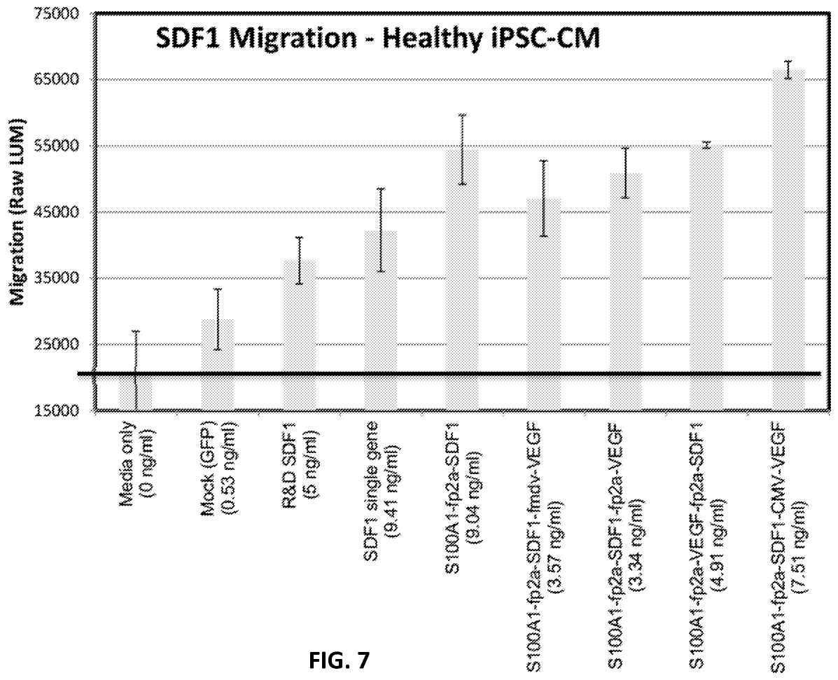

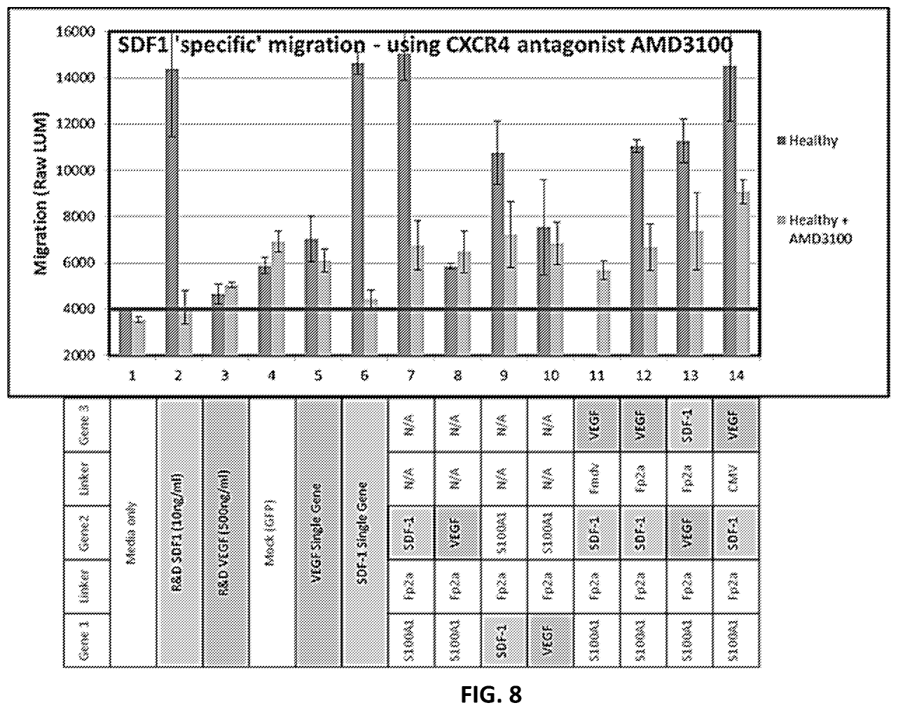

Validation of SDF1 specific migration using AMD3100 (CXCR4 antagonist) was successful. AMD3100 showed complete inhibition of SDF1-CXCR4 dependent migration (sample #2, 6). SDF1 expressed in iPSC-CMs is functional in our double gene and triple-gene constructs, as migration is reduced in presence of AMD3100 (sample #7, 12, 13). FIG. 7 and FIG. 8 show experimental data. A similar experiment performed with VEGF expressing vector constructs shows similar results, FIG. 6. Thus, these results successfully demonstrate that SDF-1α is functional (i.e., biologically active) when expressed as part of multi-genic linked constructs from dual-gene and triple-gene expression vectors when using linkers provided herein.

### Example 5A: SDF-1α Function—Jurkat Migration Assay

Transfected Jurkat Cells Produce Functional SDF1 Protein that Supports Cellular Migration

SDF-1α was examined for functionality by demonstrating CXCR4-mediated migration of two cell types: Jurkat cells and peripheral blood lymphocytes (PBLs). For Jurkat cell migration, cells in serum-free media were seeded onto the top of transwell inserts. The lower chambers were filled with conditioned media from patients (dilated, hypertrophic) or healthy iPSC-derived cardiomyocytes transfected with pXoX or single effector controls. The number of cells that migrated through the trans-well insert into the lower chamber containing the conditioned media after two hours was quantitated using a luminescence assay.

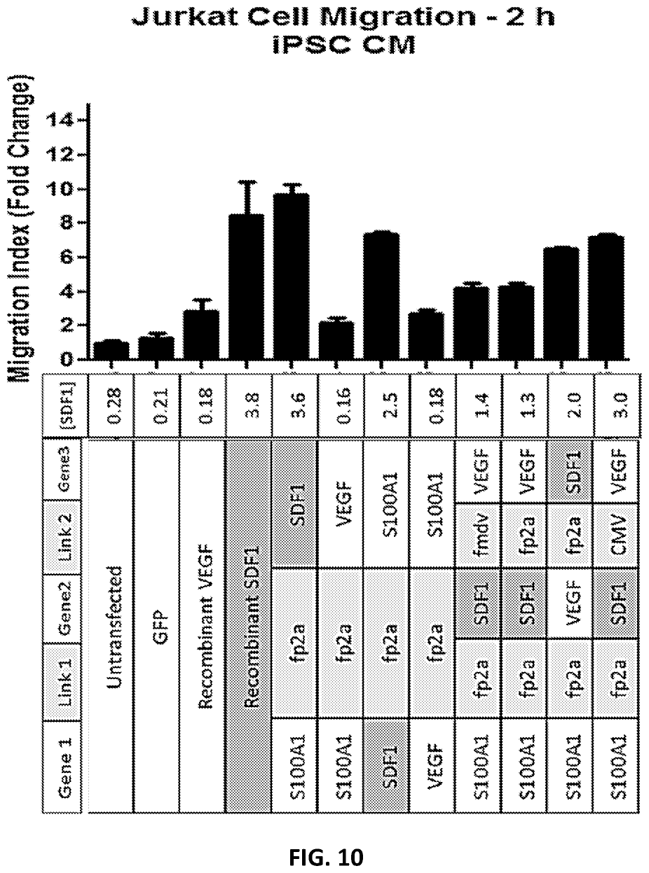

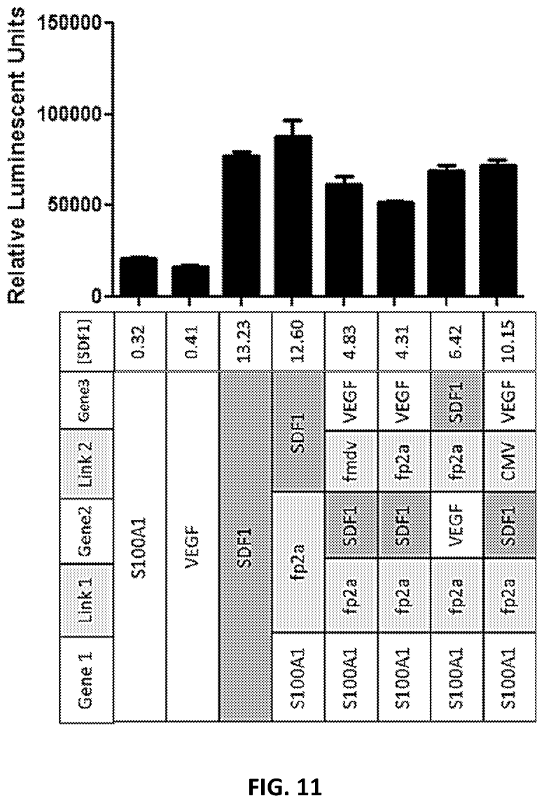

Jurkat cells were transfected with singe gene, double gene, and triple-gene gene constructs encoding for SDF1. Jurkat control cells and Jurkat exposed to AMD3100 (CXCR4 antagonist) were measured for migration efficiency 2 hours after the drug was added. FIG. 8 and FIG. 10 show experimental data. A similar experiment performed on Jurkat cells cultured with supernatant from transfected human iPSC with double gene and triple-gene vectors encoding for SDF1 show similar results, FIG. 11.

### Results

Jurkat migration towards recombinant SDF1 is CXCR4-dependent. Thus, these results successfully demonstrate that SDF-1α is functional (i.e., biologically active) when expressed as part of multi-genic linked constructs from dual-gene and triple-gene expression vectors when using linkers provided herein.

### Example 5B: Transfected Cardiomyocytes from Cardiomyopathy Patients Produce Functional SDF1 Protein that Supports Cellular Migration

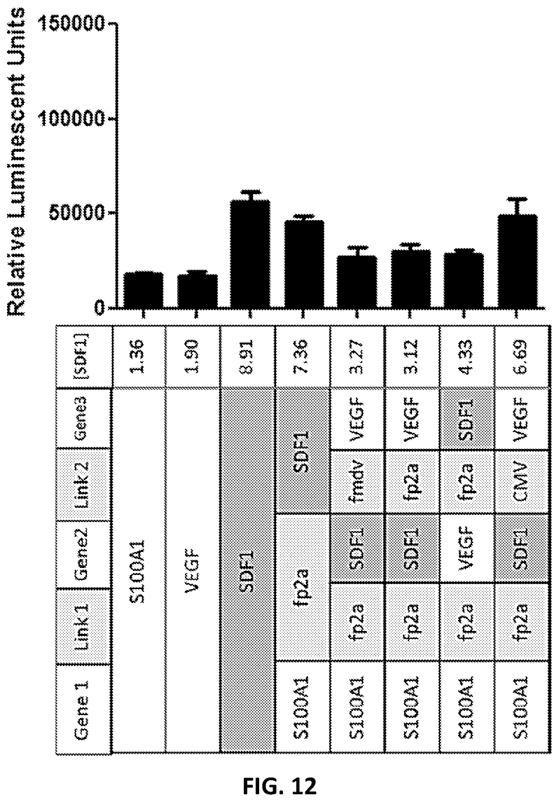

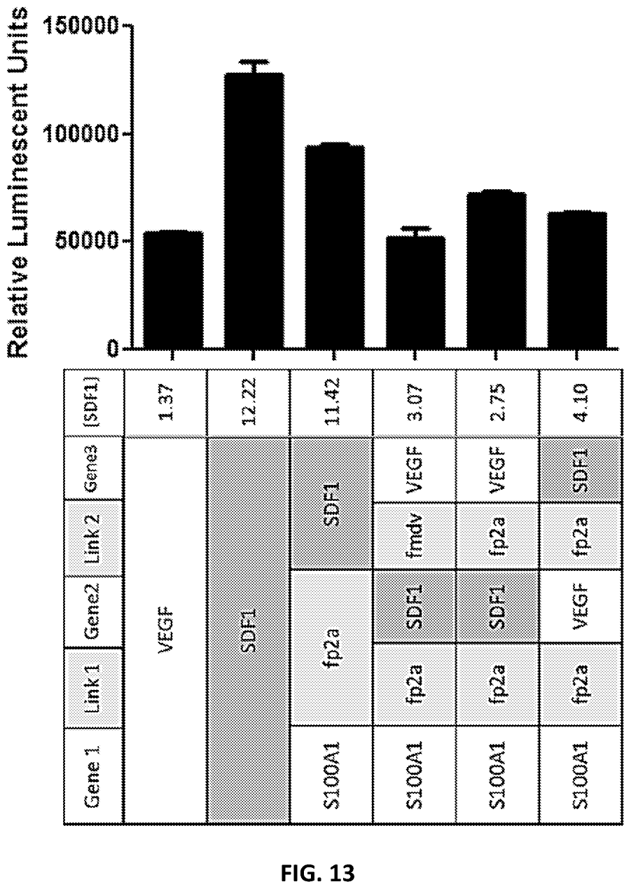

Cardiomyocyte-iPSC cells (iPSC-CM) were obtained from patients with dilated cardiomyopathy (DCM), hypertrophy cardiomyopathy (HCM) or healthy controls. iPSC cells were transfected with singe gene, dual-gene (double-gene), or triple-gene vectors encoding for S100A1 and VEGF connected by a linker described herein; S100A1, SDF1, and VEGF connected by a linker described herein; or VEGF alone. 48 hours post transfection cellular supernatant was collected. Jurkat cells were seeded on 6-well plates at a density of approximately 1:10, 1:100, and 1:1000 in the supernatant collected from the transfected iPSC cells. 96 hours following introduction of the supernatant, Jurkat cell migration was measured, FIG. 12 shows experimental data in a dilated cardiomyopathy model. FIG. 13 shows experimental data in a hypertrophy cardiomyopathy model.

### Results

iPSC-CM transfected with singe gene, dual-gene, or triple-gene vector constructs showed 4-10-fold increase in HUVEC proliferation in both cases.

### Example 6: ELISA

### Transfected 293T Cells Produce Quantifiable Protein Levels

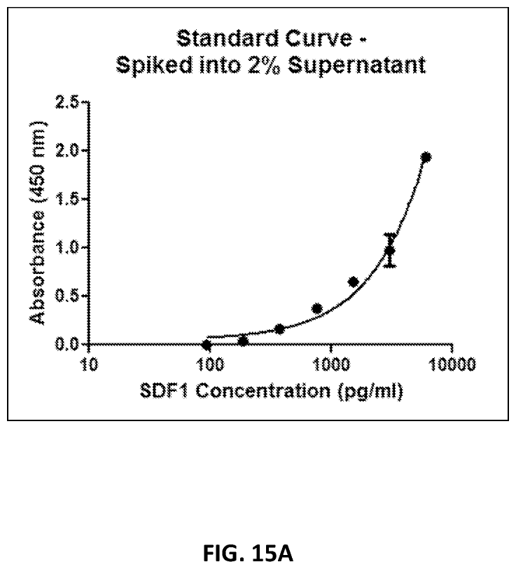

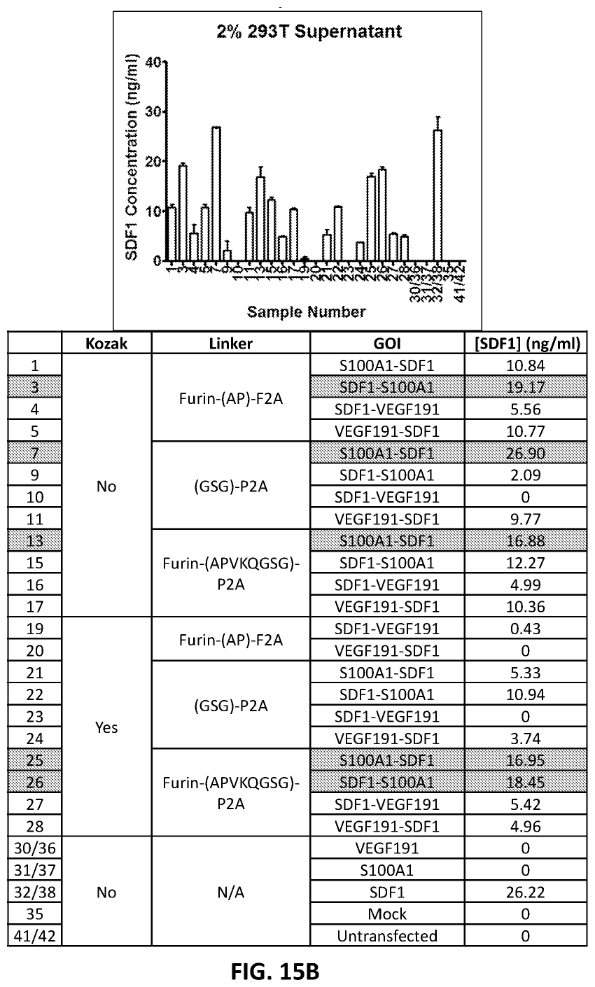

293T cells were transfected via Fugene 6 (Promega) with Xogenex single-gene, dual-gene (double-gene), triple (triple-gene) gene constructs and the appropriate control constructs. Cell supernatant and lysate were harvested at desired time point(s). 293T cells were transfected with vector constructs encoding for S100A, SDF1, VEGF191, or various combinations thereof connected by a linker described herein, utilizing vectors differing in the presence or absence of Kozak sequences, linkers (Furin-AP-F2A, GSG-P2A, Furin-APVKQGSG-P2A) (SEQ ID NO: 119), or none, as summarized in FIGS. 15-17. Concentrations of protein produced were measured by ELISA.

### Results

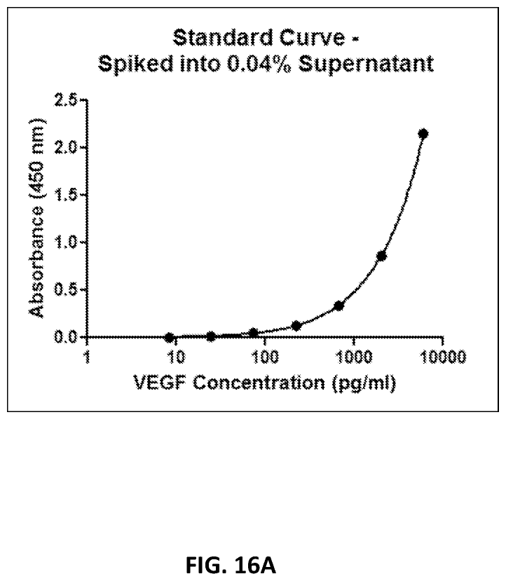

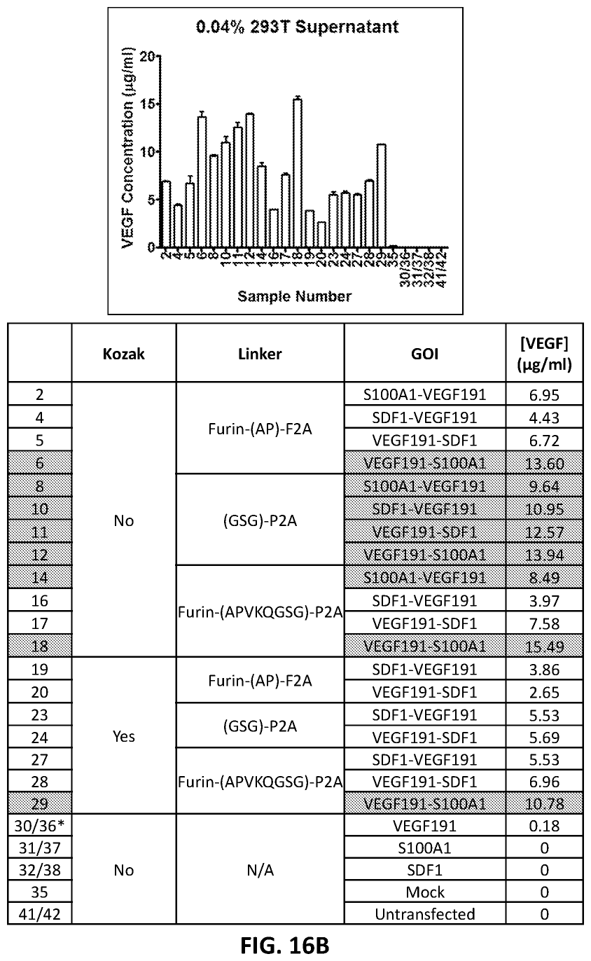

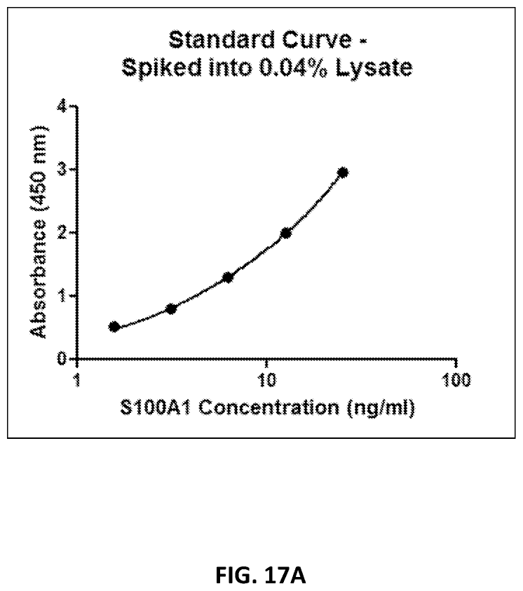

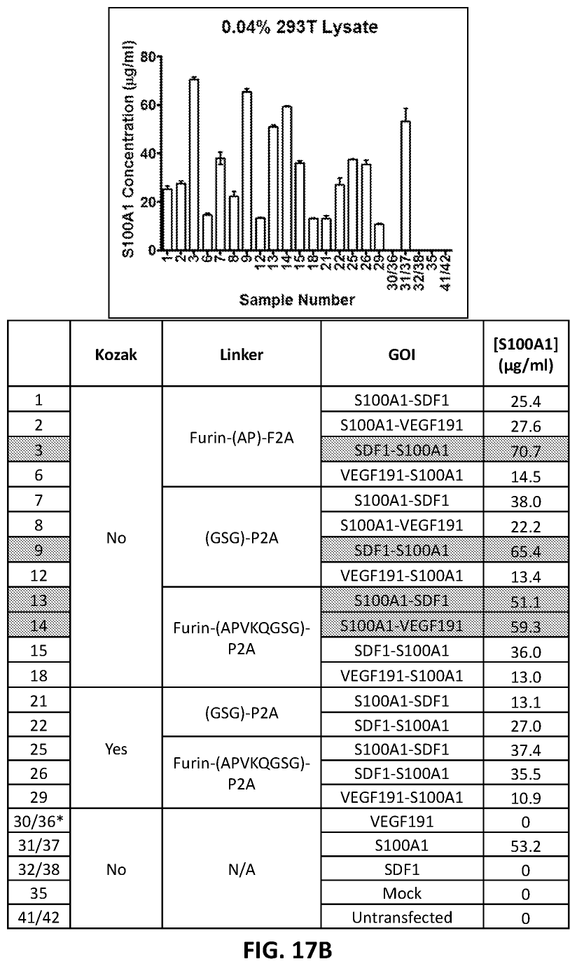

293T cells transfected with double gene vectors encoding for S100A, SDF1, VEGF191, or various combinations thereof produce measurable levels of protein. FIG. 15A and FIG. 15 B show levels of SDF1 protein. FIG. 16A and FIG. 16 B show levels of VEGF protein. FIG. 17A and FIG. 17 B show levels of S100A1 protein.

### Example 7: Triple-Gene Expression Constructs

A matrix (variety) of dual-gene (double-gene), triple-gene, and quadruple-gene (four-gene) constructs were designed as shown below in Table 5.

TABLE 5
Matrix of multi-gene plasmid construct designs

| | | | | | Optional | |
|---|---|---|---|---|---|---|
| | | | | | 2^{nd} Linker, | |
| | | | | | Promoter, or | |
| | 5′ | | | | Intervening | 3′ Optional |
| # | Promoter | Gene-1 | Linker | Gene-2 | Sequence | Gene-3 |
| 1 | CAG | S100A1 | fp2a | SDF1α | fmdv | VEGF191 |
| 2 | CAG | S100A1 | fp2a | SDF1α | p2a | VEGF191 |
| 3 | CAG | S100A1 | fp2a | VEGF191 | p2a | SDF1α |
| 4 | CAG | S100A1 | fp2a | SDF1α | fp2a | VEGF191 |
| 5 | CAG | S100A1 | fp2a | VEGF191 | fp2a | SDF1α |
| 6 | CAG | S100A1 | fp2a | SDF1α | IRES | VEGF191 |
| 7 | CAG | S100A1 | fp2a | VEGF191 | IRES | SDF1α |
| 8 | CAG | S100A1 | fp2a | SDF1α | CMV | VEGF191 |
| 9 | CAG | S100A1 | fp2a | VEGF191 | CMV | SDF1α |
| 10 | CAG | S100A1 | fp2a | SDF1α | CMV | S100A1 |
| 11 | CAG | S100A1 | fp2a | VEGF191 | CMV | S100A1 |

| *Linked to | | |
|---|---|---|
| 3′ end of | Optional 3^{rd} | Optional |
| above: | Linker | Gene-4 |
| #10 | fp2a | VEGF191 |
| #11 | fp2a | SDF1α |

*Optional 3^{rd} Linker and Gene-4 to increase S100A1 valency (i.e., increase production of S100A1 dimers)

Polypeptide expression and cleavage efficiency was tested in model cell types (i.e., 293T cells, SV40-transformed cardiomyocytes, and iPSC-derived cardiomyocytes from each of healthy donors, donors (patients) having dilated cardiomyopathy and hypertrophic cardiomyopathy. A summary of expression results, cleavage efficiency results, and expression considerations for various linker (i.e., linking sequence) combinations is shown below in Table 6.

TABLE 6
Polypeptide Cleavage/Linker/Gene of Interest (GOI) Expression Differentials

| | GOI Polypeptide | GOI Polypeptide/ | | |
|---|---|---|---|---|
| | Expression | Linker Cleavage | Consider | Overall |
| Linker | Levels | (Western Blot/ | Biological | Performance |
| Composition | (ELISA Assay) | Immunoblot Assay) | Aspects of: | Ranking |
| f2pa-fp2a | High | Complete cleavage | Presence of 2A | #1 |
| | | all 3 GOIs | C-terminal tail | |
| f2pa-fmdv | High | Complete cleavage | Presence of 2A | #2 |
| | | all 3 GOIs | C-terminal tail | |
| f2pa-CMV | High | Complete cleavage | Differing stoichiometric | #3 |
| | | all 3 GOIs | expression ratios of GOIs | |
| | | | and promoter silencing | |
| f2pa-CMV-fp2a | High | Complete cleavage | Differing stoichiometric | #4 |
| | | all 3 GOIs | expression ratios of GOIs | |
| | | | and promoter silencing | |
| f2pa-p2a | Very high | Poor cleavage between | n/a | n/a |
| | | 2^{nd} and 3^{rd} GOIs | | |
| f2pa-IRES | Poor (3^{rd} GOI; | n/a | n/a | n/a |
| | 3′ of IRES) | | | |

Lead Construct Configurations:
(5′)-CAG Promoter-S100A1-fp2a (linker)-SDF1α-fp2a (linker)-VEGF191-(3′)
(5′)-CAG Promoter-S100A1-fp2a (linker)-VEGF191-fp2a (linker)-SDF1α-(3′)

ELISA Assay—Expression of S100A1 from Triple-Gene Constructs

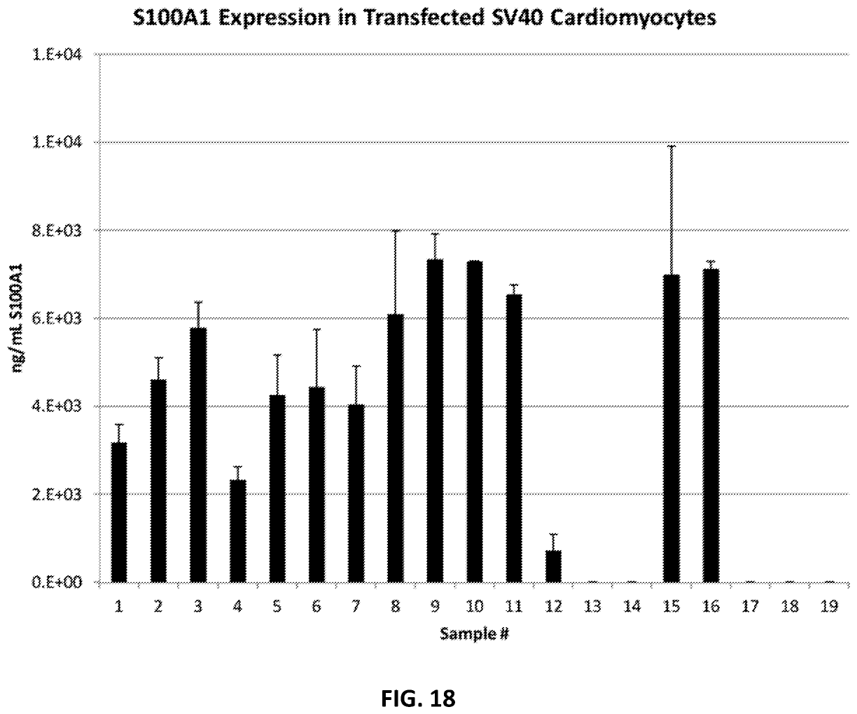

ELISA assays were performed to quantify expression of S100A1 from triple-gene pXoX constructs in comparison to single-gene pXoX controls and previously identified lead candidate, dual-gene constructs. SV40 transformed cardiomyocytes were transfected with each of the test plasmid constructs and cell supernatants and lysates were harvested at desired time point(s). The cardiomyocytes were transfected with vector constructs encoding for S100A, SDF1α, VEGF191, or various combinations thereof connected by linker sequences described herein and indicated below in Table 7 as respective first, second, and third linker sequences (e.g., fp2a, fmdv, p2a, IRES, CMV, or no linking sequence). Concentrations of protein produced were measured by ELISA. See, FIG. 18; sample numbers correspond to sample numbers shown in Table 7 below.

Configuration of constructs and S100A1 expression results are shown in Table 7 (below).

TABLE 7
S100A1 Polypeptide Expression From Triple-
Gene Compared To Other Constructs.

| | | | | S100A1 |
|---|---|---|---|---|
| Sample | Name | Linker | GOI | ng/mL |
| 1 | Triple 1 | fp2a-fmdv | S100A1-SDF1-VEGF | 3176 |
| 2 | Triple 2 | fp2a-p2a | S100A1-SDF1-VEGF | 4616 |
| 3 | Triple 3 | fp2a-p2a | S100A1-VEGF-SDF1 | 5792 |
| 4 | triple 4 | fp2a-fp2a | S100A1-SDF1-VEGF | 2335 |
| 5 | Triple 5 | fp2a-fp2a | S100A1-VEGF-SDF1 | 4258 |
| 6 | Triple 6 | fp2a-IRES | S100A1-SDF1-VEGF | 4440 |
| 7 | Triple 7 | fp2a-IRES | S100A1-VEGF-SDF1 | 4042 |
| 8 | Triple 8 | fp2a-CMV | S100A1-SDF1-VEGF | 6096 |
| 9 | Triple 9 | fp2a-CMV | S100A1-VEGF-SDF1 | 7342 |
| 10 | Triple 10 | fp2a-CMV- | S100A1-SDF1- | 7290 |
| | | fp2a | S100A1-VEGF | |
| 11 | Triple 11 | fp2a-CMV- | S100A1-VEFG- | 6541 |
| | | fp2a | S100A1-SDF1 | |
| 12 | Single Gene | N/A | S100A1 | 724 |
| | Control | | | |
| 13 | Single Gene | N/A | SDF1 | 0 |
| | Control | | | |
| 14 | Single Gene | N/A | VEGF | 0 |
| | Control | | | |
| 15 | Dual Lead | fp2a | S100A1-SDF1 | 6988 |
| | #13 | | | |
| 16 | Dual Lead | fp2a | S100A1-VEGF | 7121 |
| | #14 | | | |
| 17* | Dual Lead | fp2a | SDF1-S100A1 | 0 |
| | #15 | | | |
| 18 | GFP | N/A | GFP | 0 |
| 19 | Untrans- | N/A | N/A | 0 |
| | fected | | | |

ELISA Assay—Expression of SDF1α from Triple-Gene Constructs

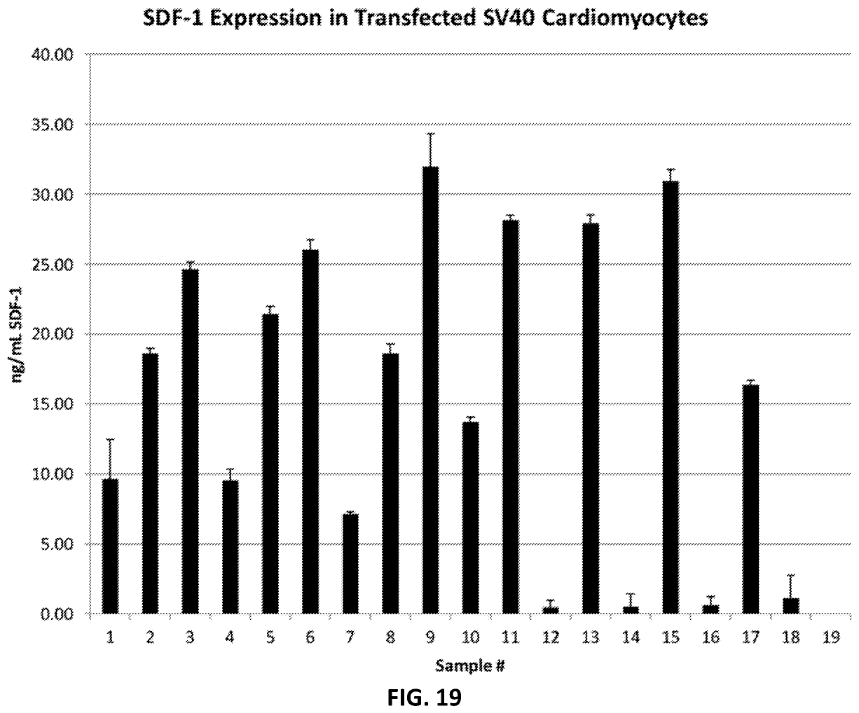

ELISA assays were performed to quantify expression of SDF1α□from triple-gene pXoX constructs in comparison to single-gene pXoX controls and previously identified lead candidate, dual-gene constructs. SV40 transformed cardiomyocytes were transfected with each of the test plasmid constructs and cell supeatants and lysates were harvested at desired time point(s). The cardiomyocytes were transfected with vector constructs encoding for S100A, SDF1α, VEGF191, or various combinations thereof connected by linker sequences described herein and indicated below in Table 8 as respective first, second, and third linker sequences (e.g., fp2a, fmdv, p2a, IRES, CMV, or no linking sequence). Concentrations of protein produced were measured by ELISA. See, FIG. 19; sample numbers correspond to sample numbers shown in Table 8 below.

TABLE 8
SDF1α Polypeptide Expression From
Triple-Gene Compared To Other Constructs.

| | | | | SDF1α |
|---|---|---|---|---|
| Sample | Name | Linker | GOI | ng/mL |
| 1 | Triple 1 | fp2a-fmdv | S100A1-SDF1-VEGF | 9.66 |
| 2 | Triple 2 | fp2a-p2a | S100A1-SDF1-VEGF | 18.63 |
| 3 | Triple 3 | fp2a-p2a | S100A1-VEGF-SDF1 | 24.65 |
| 4 | Triple 4 | fp2a-fp2a | S100A1-SDF1-VEGF | 9.55 |
| 5 | Triple 5 | fp2a-fp2a | S100A1-VEGF-SDF1 | 21.45 |
| 6 | Triple 6 | fp2a-IRES | S100A1-SDF1-VEGF | 26.07 |
| 7 | Triple 7 | fp2a-IRES | S100A1-VEGF-SDF1 | 7.16 |
| 8 | Triple 8 | fp2a-CMV | S100A1-SDF1-VEGF | 18.64 |
| 9 | Triple 9 | fp2a-CMV | S100A1-VEGF-SDF1 | 31.99 |
| 10 | Triple 10 | fp2a-CMV- | S100A1-SDF1- | 13.73 |
| | | fp2a | S100A1-VEGF | |
| 11 | Triple 11 | fp2a-CMV- | S100A1-VEFG- | 28.17 |
| | | fp2a | S100A1-SDF1 | |
| 12 | Single Gene | N/A | S100A1 | 0.46 |
| | Control | | | |
| 13 | Single Gene | N/A | SDF1 | 27.95 |
| | Control | | | |
| 14 | Single Gene | N/A | VEGF | 0.51 |
| | Control | | | |
| 15 | Dual Lead | fp2a | S100A1-SDF1 | 30.94 |
| | #13 | | | |
| 16 | Dual Lead | fp2a | S100A1-VEGF | 0.64 |
| | #14 | | | |
| 17 | Dual Lead | fp2a | SDF1-S100A1 | 16.40 |
| | #15 | | | |
| 18 | GFP Control | N/A | GFP | 1.12 |
| 19 | Untrans- | N/A | N/A | 0 |
| | fected | | | |

ELISA Assay—Expression of VEGF191 from Triple-Gene Constructs

ELISA assays were performed to quantify expression of VEGF191 from triple-gene pXoX constructs in comparison to single-gene pXoX controls and previously identified lead candidate, dual-gene constructs. SV40 transformed cardiomyocytes were transfected with each of the test plasmid constructs and cell supernatants and lysates were harvested at desired time point(s). The cardiomyocytes were transfected with vector constructs encoding for S100A, SDF1α, VEGF191, or various combinations thereof connected by linker sequences described herein and indicated below in Table 9 as respective first, second, and third linker sequences (e.g., fp2a, fmdv, p2a, IRES, CMV, or no linking sequence). Concentrations of protein produced were measured by ELISA. See, FIG. 20; sample numbers correspond to sample numbers shown in Table 9 below.

TABLE 9
VEGF191 Polypeptide Expression From Triple-
Gene Compared To Other Constructs.

| | | | | VEGF191 |
|---|---|---|---|---|
| Sample | Name | Linker | GOI | ng/mL |
| 1 | Triple 1 | fp2a-fmdv | S100A1-SDF1-VEGF | 197.1 |
| 2 | Triple 2 | fp2a-p2a | S100A1-SDF1-VEGF | 281.9 |
| 3 | Triple 3 | fp2a-p2a | S100A1-VEGF-SDF1 | 169.6 |
| 4 | Triple 4 | fp2a-fp2a | S100A1-SDF1-VEGF | 73.1 |
| 5 | Triple 5 | fp2a-fp2a | S100A1-VEGF-SDF1 | 99.9 |
| 6 | Triple 6 | fp2a-IRES | S100A1-SDF1-VEGF | 24.0 |
| 7 | Triple 7 | fp2a-IRES | S100A1-VEGF-SDF1 | 111.2 |
| 8 | Triple 8 | fp2a-CMV | S100A1-SDF1-VEGF | 226.5 |
| 9 | Triple 9 | fp2a-CMV | S100A1-VEGF-SDF1 | 149.4 |
| 10 | Triple 10 | fp2a-CMV- | S100A1-SDF1- | 231.0 |
| | | fp2a | S100A1-VEGF | |
| 11 | Triple 11 | fp2a-CMV- | S100A1-VEFG- | 239.6 |
| | | fp2a | S100A1-SDF1 | |
| 12 | Single Gene | N/A | S100A1 | 1.5 |
| | Control | | | |
| 13 | Single Gene | N/A | SDF1 | 1.7 |
| | Control | | | |
| 14 | Single Gene | N/A | VEGF | 427.5 |
| | Control | | | |
| 15 | Dual Lead | fp2a | S100A1-SDF1 | 1.1 |
| | #13 | | | |
| 16 | Dual Lead | fp2a | S100A1-VEGF | 265.3 |
| | #14 | | | |
| 17 | Dual Lead | fp2a | SDF1-S100A1 | 1.4 |
| | #15 | | | |
| 18 | GFP Control | N/A | GFP | 1.1 |
| 19 | Untrans- | N/A | N/A | 5.5 |
| | fected | | | |

Western Blot (Immunoblot) Assays Verify Efficient Cleavage of S100A1, SDF-1α and VEGF191 from Triple-Gene Constructs

Confirmation of the appropriate size and sequence of individual effector proteins within the relevant cell types was demonstrated by immunoblot and mass spectrometry analyses (as described further herein). Multiple pertinent cell lines were transfected with pXoX and either cell lysates (for S100A1) or conditioned media (for SDF-1α and VEGF165) were analyzed. Controls for immunoblot analyses were lysates/conditioned media from cells transfected with the appropriate single effector (pS100A1, pSDF-1α, pVEGF165) and the corresponding commercially available, purified, recombinant protein.

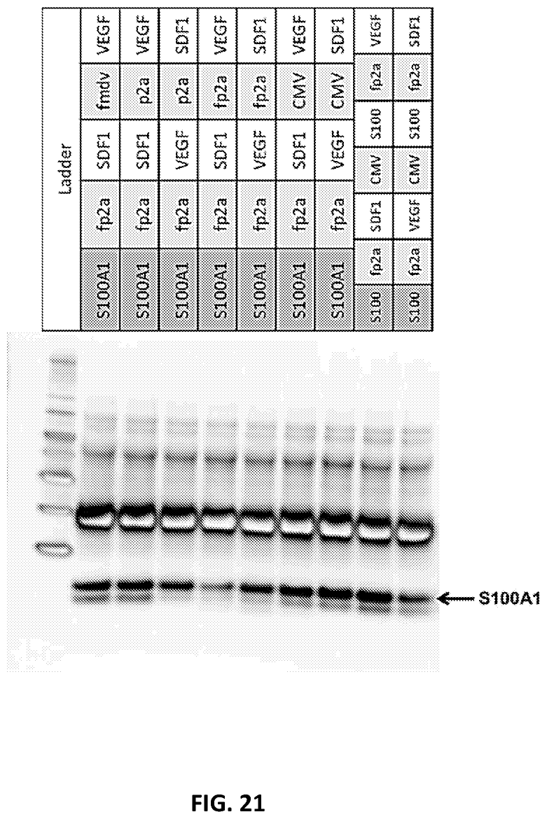

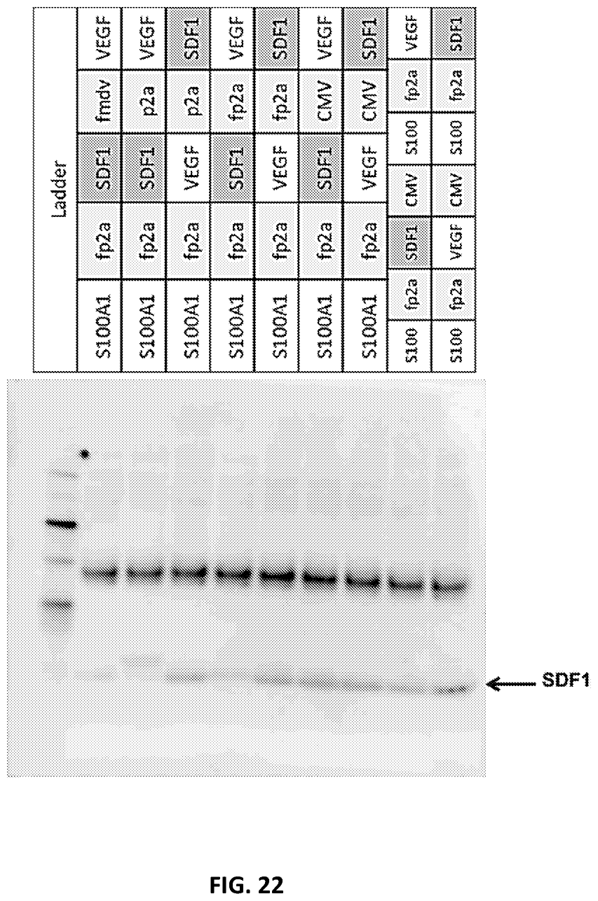

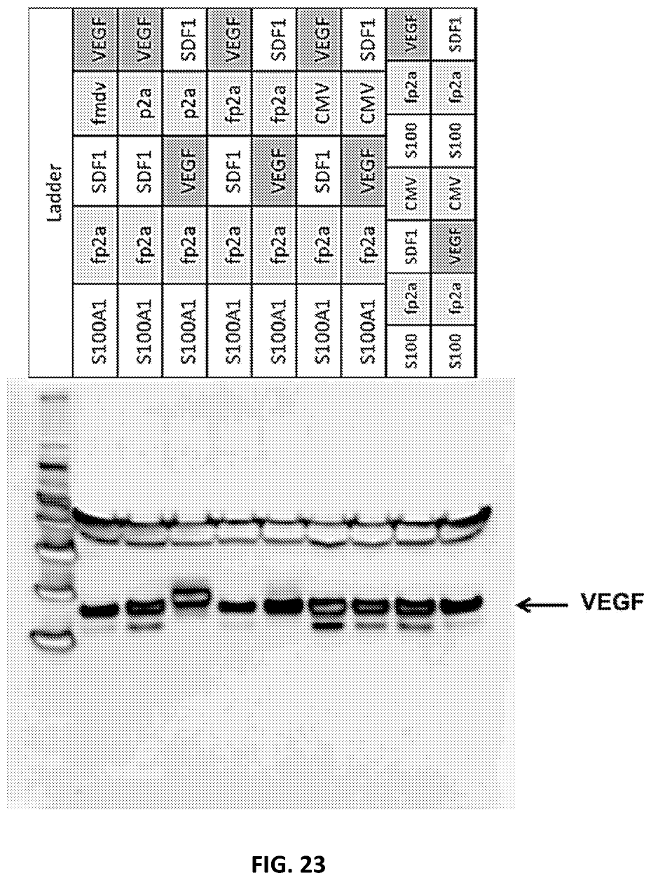

SDS-PAGE/immunoblot results were consistent across all tested cell lines (293T, SV40 immortalized cardiomyocytes, iPSC-derived cardiomyocytes from healthy individual, dilated cardiomyopathy patient, hypertrophy cardiomyopathy patient). Immunoblot data from SV40 immortalized cardiomyocytes is shown below in FIGS. 21-23 with arrows indicating the expected size of each of the indicated effector proteins.

For each GOI, immunoblot analysis was performed using standard procedures according to manufacturer's instructions. Detection reagent conditions were as follows:
S100A1 detection—Anti-S100A1 antibody (R&D Systems, Inc.) diluted 1:200, incubated with blot overnight at 4 degrees C.; anti-sheep HRP (horse radish peroxidase) antibody (KPL Inc., Gaithersburg, MD, USA) diluted 1:20,000, incubated with blot at room temperature (RT) 30 minutes; signal detection via LUMIGLO® Ultra (KPL Inc.);
SDF-1α detection—Anti-SDF-1α antibody (R&D Systems, Inc.) diluted 1:2000, incubated with blot overnight at 4 degrees C.; anti-goat HRP (Jackson ImmunoResearch Laboratories, Inc., West Grov, PA, USA) antibody diluted 1:20,000, incubated with blot at room temperature (RT) 30 minutes; signal detection via LUMIGLO® Ultra (KPL Inc.).
VEGF detection—Anti-VEGF antibody (Abcam, Plc., Cambridge, MA) diluted 1:2000, incubated with blot overnight at 4 degrees C.; anti-rabbit HRP (Jackson ImmunoResearch Laboratories, Inc.) antibody diluted 1:20,000, incubated with blot at room temperature (RT) 30 minutes; signal detection via LUMIGLO® Ultra (KPL Inc.).

Example immunoblots are shown in FIGS. 21, 22 and 23.

TABLE 10
Samples from cells expressing the various constructs are shown in lanes 1-9 (lane “L”
showing a polypeptide molecular weight control ladder (220, 120, 100,
80, 60, 50, 40, 30, 20, 14, 6, and 3 kDa markers)).

| Lane | GOI | Linker | GOI | Linker | GOI | Linker | GOI |
|---|---|---|---|---|---|---|---|
| L | Ladder - Molecular Weight Marker | | | | | | |
| 1 | S100A1 | fp2a | SDF1α | fmdv | VEGF191 | | |
| 2 | S100A1 | fp2a | SDF1α | p2a | VEGF191 | | |
| 3 | S100A1 | fp2a | VEGF191 | p2a | SDF1α | | |
| 4 | S100A1 | fp2a | SDF1α | fp2a | VEGF191 | | |
| 5 | S100A1 | fp2a | VEGF191 | fp2a | SDF1α | | |
| 6 | S100A1 | fp2a | SDF1α | CMV | VEGF191 | | |
| 7 | S100A1 | fp2a | VEGF191 | CMV | SDF1α | | |
| 8 | S100A1 | fp2a | SDF1α | CMV | S100A1 | fp2a | VEGF191 |
| 9 | S100A1 | fp2a | VEGF191 | CMV | S100A1 | fp2a | SDF1α |

Results indicate that each of the three GOIs are cleaved efficiently as expressed from triple-gene constructs.

Mass Spectometry Assay—Multi-Gene Expression Products were Evaluated by Mass Spectrometry.

Cleavage products of lead candidates were verified using mass spectrometry. Trypsin, chymotrypsin and elastase digestion was performed using standard procedures. Each gel digest was further analyzed by nano LC/MS/MS* with a Waters NanoAcquity HPLC system (Waters Corp., Milford, MA, USA) interfaced to a ThermoFisher Q Exactive HF (ThermoFisher Scientific, Waltham, MA, USA). Data was processed using Mascot database. Mascot DAT files were parsed into the Scaffold software for validation, filtering and to create a nonredundant list per sample. Data were filtered using a minimum protein value of 90%, a minimum peptide value of 50% (Prophet scores) and requiring at least two unique peptides per protein. Consistent with the above described Western blot (immunoblot) analysis, the mass spectrometry results further verified proteolytic cleavage of S100A1, SDF-1 and VEGF when expressed from lead dual-gene and triple-gene constructs; see, Table 11. *LC/MS/MS indicates liquid chromatography (LC) with mass spectrometry (MS). MS/MS is a combination of two mass analyzers in one mass spectrometry instrument. The first MS filters for precursor ions followed by a fragmentation of the precursor ion with high energy and, for example, nitrogen gas. A second mass analyzer then filters for product ions generated by the fragmentation. Advantage of MS/MS is increased sensitivity.

TABLE 11
Summary of Polypeptide Cleavage Analyzed by Mass
Spectrometry for Lead Candidate Constructs

| | Construct Configuration | | | |
|---|---|---|---|---|
| | (Optional promoter | | | |
| | type)-GOI#1-linker- | S100A1 | SDF | VEGF |
| | GOI#2-linker-GOI#3 | Cleavage | Cleavage | Cleavage |
| Lead Dual | (CAG)-S100A1-fp2a- | Yes | Yes | n/a |
| #13 | SDF1 | | | |
| Lead Dual | (CAG)-S100A1-fp2a- | Yes | n/a | Yes |
| #14 | VEGF | | | |
| Triple #1 | (CAG)-S100A1-fp2a- | Yes | Yes | Yes |
| | SDF1-fmdv-VEGF | | | |
| Triple #2 | (CAG)-S100A1-fp2a- | Yes | Yes | Yes |
| | SDF1-p2a-VEGF | | | |
| Triple #3 | (CAG)-S100A1-fp2a- | Yes | Yes | Yes |
| | SDF1-CMV-VEGF | | | |
| Triple #4 | (CAG)-S100A1-fp2a- | Yes | Yes | Yes |
| | SDF1-fp2a-VEGF | | | |
| Triple #5 | (CAG)-S100A1-fp2a- | Yes | Yes | Yes |
| | VEGF-fp2a-SDF1 | | | |

Mass spectrometry (MS) was also used to assess the presence or absence of linker amino acid residues at the end (C-terminus) of each GOI. MS was performed as indicated above. Results are shown below in Table 12.

TABLE 12
MS Analysis of C-terminal Linker Amino Acid
Residues for Lead Candidate Constructs

| | Construct Configuration | | | |
|---|---|---|---|---|
| | (Optional promoter | Presence of Linker AA | | |
| | type)-GOI#1-linker- | residues (C-termini)?* | | |
| | GOI#2-linker-GOI#3 | S100A1 | SDF | VEGF |
| Dual Leads | (CAG)-S100A1-fp2a- | Yes | no | n/a |
| | SDF1 | | | |
| | (CAG)-S100A1-fp2a- | Yes | n/a | no |
| | VEGF | | | |
| Primary Triple | (CAG)-S100A1-fp2a- | Yes | no | no |
| Leads | SDF1-fp2a-VEGF | | | |
| | (CAG)-S100A1-fp2a- | Yes | no | Yes |
| | VEGF-fp2a-SDF1 | | | |
| Secondary Triple | (CAG)-S100A1-fp2a- | Yes | no | no |
| Leads | SDF1-fmdv-VEGF | | | |
| | (CAG)-S100A1-fp2a- | Yes | no | no |
| | SDF1-CMV-VEGF | | | |
| Tertiary Triple | (CAG)-S100A1-CMV- | n/a | no | no |
| Leads | SDF1-fp2a-VEGF | | | |
| | (CAG)-S100A1-CMV- | n/a | no | Yes |
| | VEGF-fp2a-SDF1 | | | |

*“Yes” indicates presence of > (greater than) 2 amino acid (AA) residues on C-terminal tail (which may be referred to herein as a ”linker tail”); “No” indicates presence of < or = (less than or equal to) 2A on C-terminal tail.

Western blot (Immunoblot) Analysis of S100A1 Polypeptide Dimerization

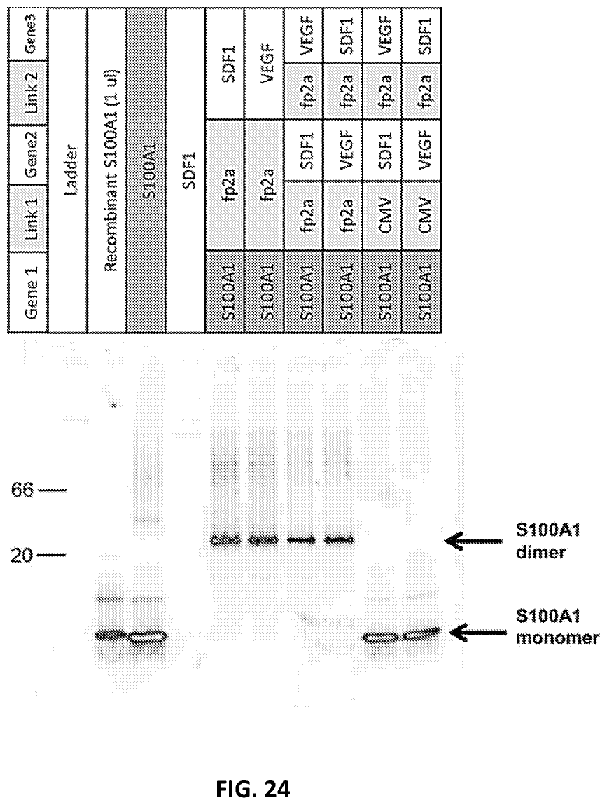

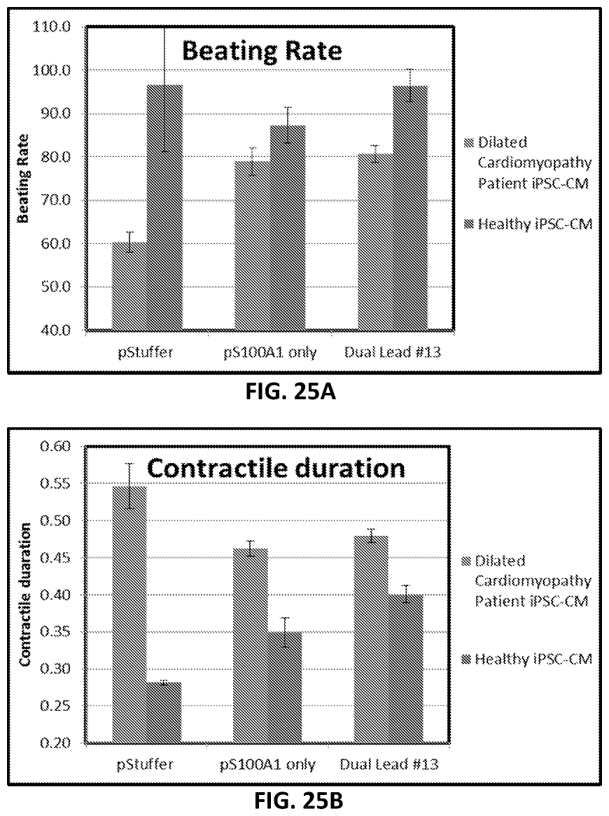

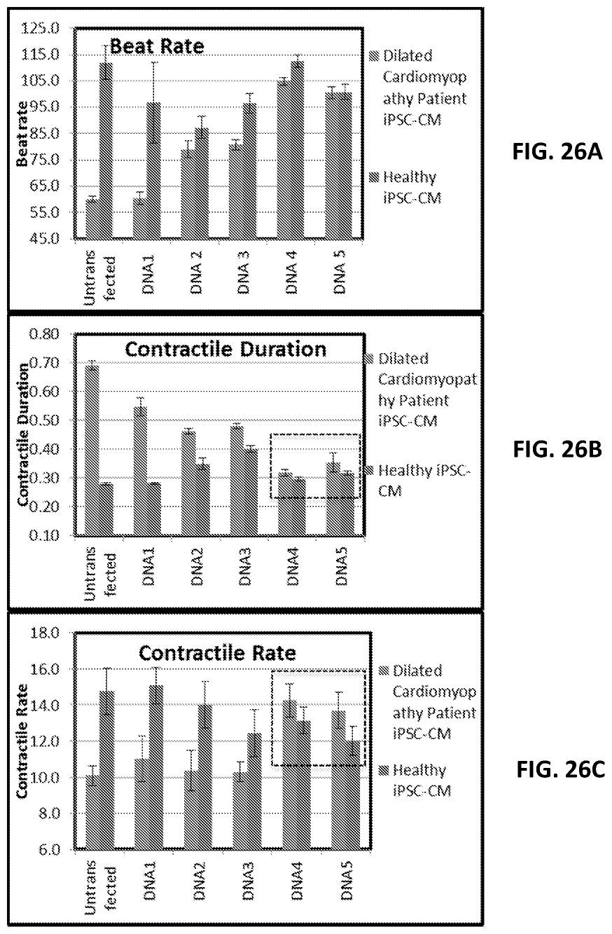

Non-denaturing PolyAcrylamide Gel Electrophoresis (PAGE) was performed to assess dimer formation of S100A1 polypeptides expressed from dual-gene and triple gene constructs. Conditioned media of transfected 293T cells was analyzed by native-PAGE/immunoblot for monomeric and dimeric levels of S100A1. Results demonstrate that the S100A1 cleaved from lead dual and lead triple candidates is predominantly in a dimeric form, whereas S100A1 expressed by the single effector construct pS100A1 and by triple-gene constructs having a CMV linker C-terminal to S100A1 is predominantly monomeric; see, FIG. 24.

Notably, a unique aspect of the present invention is provided and enabled by the specific sequential order of polypeptides encoded by constructs of the invention. Thus, in one aspect of the invention, specifically placing S100A1 protein first achieves and allows functional expression of biologically active S100A1 molecules because the remaining 2A tail on S100A1 was, surprisingly, discovered not to interfere with S100A1 biological activity. In contrast, it was discovered that placing S100A1 as the second gene resulted in incomplete cleavage of genes expressed via the construct.

Accordingly, analysis of pXoX expression using mass spectrometry, as indicated below in Table 13 showed that S100A1 was the only effector that did not exhibit furin cleavage resulting in removal of the C-terminal 2A peptide. Hence, there is no 2A C-terminal tail left on SDF1a or VEGF. Only S100A1 has a 2A linker-tail present. The exact ending amino acid sequence is PG. It cleaves between G and P of ending PGP sequence. Hence, the C-terminal linker tail on S100A1 as expressed from pXoX comprises the sequence (from N- to C-terminus) S101A1 polypeptide fused to—RAKRAPVKQGSGATNFSLLKQAGDVEENPG (SEQ ID NO: 123); which may be referred to herein as the “2A tail”. For example, in one embodiment, from the initial methionine of S100A1 to the C-terminal end of the fp2a cleaved linker tail a first GOI of the invention comprises the sequence:

(SEQ ID NO: 124)
MGSELETAMETLINVFHAHSGKEGDKYKLSKKELKELLQTELSGFLDAQK
DVDAVDKVMKELDENGDGEVDFQEYVVLVAALTVACNNFFWENSRAKRAP
VKQGSGATNFSLLKQAGDVEENPG.

However, as provided via data presented herein, it has been demonstrated that this S100A1 tail is ‘inert’—i.e. does not affect S100A1 function (biological activity).

TABLE 13
Mass Spectrometry Analyses of Proteins Expressed From pXoX

| Effectors in | Cellular | Expected proteolytic | Absence of |
|---|---|---|---|
| pXoX | location | cleavage | 2A Tail |
| S100A1 | Intracellular | Yes | No |
| SDF-1α | Secreted | Yes | Yes |
| VEGF165 | Secreted | Yes | Yes |

### Example 8: Triple-Gene Vector Transfection

293T cells were plated at 750,000 cells per well in a 6 well plate and incubated overnight at plates to 37° C./5% CO₂ incubator overnight. The following day, the cells were transfected using FuGENE 6 (Promega) with single (singe-gene), dual (double-gene), triple (triple-gene), Table 14, gene constructs and the appropriate control constructs using plasmid vector at 100 ng/ml diluted in OptiMEM media (LifeTechnologies). After transfection, the cells were incubated. Cell supernatant and lysate were harvested at desired at 72 hours.

TABLE 14
pXoX Triple-Gene Vector Sequence

| SEQ ID No. | Construct | Sequence |
|---|---|---|
| 108 | pXoX | TAACTATAACGGTCCTAAGGTAGCGACGTACGAACCGTTGGGCGCGCCTGGGGA |
| | | TAGCGATCGCTGCTGGCGCGGTCCGCTATGAGGTCTCTGATAGACCACAGACGC |
| | | GTCGACATTGATTATTGACTAGTTATTAATAGTAATCAATTACGGGGTCATTAG |
| | | TTCATAGCCCATATATGGAGTTCCGCGTTACATAACTTACGGTAAATGGCCCGC |
| | | CTGGCTGACCGCCCAACGACCCCCGCCCATTGACGTCAATAATGACGTATGTTC |
| | | CCATAGTAACGCCAATAGGGACTTTCCATTGACGTCAATGGGTGGAGTATTTAC |
| | | GGTAAACTGCCCACTTGGCAGTACATCAAGTGTATCATATGCCAAGTACGCCCC |
| | | CTATTGACGTCAATGACGGTAAATGGCCCGCCTGGCATTATGCCCAGTACATGA |
| | | CCTTATGGGACTTTCCTACTTGGCAGTACATCTACGTATTAGTCATCGCTATTA |
| | | CCATGGTCGAGGTGAGCCCCACGTTCTGCTTCACTCTCCCCATCTCCCCCCCCT |
| | | CCCCACCCCCAATTTTGTATTTATTTATTTTTTAATTATTTTGTGCAGCGATGG |
| | | GGGCGGGGGGGGGGGGGGGGCGCGCGCCAGGCGGGGCGGGGCGGGGCGAGGGGC |
| | | GGGGCGGGGCGAGGCGGAGAGGTGCGGCGGCAGCCAATCAGAGCGGCGCGCTCC |
| | | GAAAGTTTCCTTTTATGGCGAGGCGGCGGCGGCGGCGGCCCTATAAAAAGCGAA |
| | | GCGCGCGGCGGGCGGGAGTCGCTGCGCGCTGCCTTCGCCCCGTGCCCCGCTCCG |
| | | CCGCCGCCTCGCGCCGCCCGCCCCGGCTCTGACTGACCGCGTTACTCCCACAGG |
| | | TGAGCGGGCGGGACGGCCCTTCTCCTCCGGGCTGTAATTAGCGCTTGGTTTAAT |
| | | GACGGCTTGTTTCTTTTCTGTGGCTGCGTGAAAGCCTTGAGGGGCTCCGGGAGG |
| | | GCCCTTTGTGCGGGGGGAGCGGCTCGGGGGGTGCGTGCGTGTGTGTGTGCGTGG |
| | | GGAGCGCCGCGTGCGGCTCCGCGCTGCCCGGCGGCTGTGAGCGCTGCGGGCGCG |
| | | GCGCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGGTG |
| | | TGTGCGTGGGGGGGTGAGCAGGGGGTGTGGGCGCGTCGGTCGGGCTGCAACCCC |
| | | CCCTGCACCCCCCTCCCCGAGTTGCTGAGCACGGCCCGGCTTCGGGTGCGGGGC |
| | | TCCGTACGGGGCGTGGCGCGGGGCTCGCCGTGCCGGGCGGGGGGTGGCGGCAGG |
| | | TGGGGGTGCCGGGCGGGGCGGGGCCGCCTCGGGCCGGGGAGGGCTCGGGGGAGG |
| | | GGCGCGGCGGCCCCCGGAGCGCCGGCGGCTGTCGAGGCGCGGCGAGCCGCAGCC |
| | | ATTGCCTTTTATGGTAATCGTGCGAGAGGGCGCAGGGACTTCCTTTGTCCCAAA |
| | | TCTGTGCGGAGCCGAAATCTGGGAGGCGCCGCCGCACCCCCTCTAGCGGGCGCG |
| | | GGGCGAAGCGGTGCGGCGCCGGCAGGAAGGAAATGGGCGGGGAGGGCCTTCGTG |
| | | CGTCGCCGCGCCGCCGTCCCCTTCTCCCTCTCCAGCCTCGGGGCTGTCCGCGGG |
| | | GGGACGGCTGCCTTCGGGGGGGACGGGGCAGGGCGGGGTTCGGCTTCTGGCGTG |
| | | TGACCGGCGGCTCTAGAGCCTCTGCTAACCATGTTCATGCCTTCTTCTTTTTCC |
| | | TACAGCTCCTGGGCAACGTGCTGGTTATTGTGCTGTCTCATCATTTTGGCAAAG |
| | | AATTCCCTGCAGGAAATTGAGCCCGCAGCCTCCCGCTTCGCTCTCTGCTCCTCC |
| | | TGTTCGACAGTCAGCCGCATCTTCTTTTGCGTCGCCAGCCGAGCCACATCGCTC |
| | | AGACACCGCTAGCATGGGCAGCGAACTGGAAACCGCCATGGAGACTTTGATAAA |
| | | TGTTTTCCACGCGCATAGCGGCAAAGAAGGGGACAAGTACAAGCTGTCAAAAAA |
| | | GGAGCTGAAAGAACTGCTGCAGACCGAATTGAGCGGCTTCCTGGACGCTCAGAA |
| | | AGATGTCGATGCCGTCGACAAAGTGATGAAAGAGCTTGACGAGAACGGTGACGG |
| | | TGAAGTCGATTTTCAGGAATATGTGGTGCTGGTGGCCGCCCTTACTGTAGCATG |
| | | CAACAATTTCTTTTGGGAAAATTCACGTGCAAAGCGTGCACCGGTGAAACAGGG |
| | | AAGCGGAGCTACTAACTTCAGCCTGCTGAAGCAGGCTGGAGACGTGGAGGAGAA |
| | | CCCTGGACCTATGAATGCCAAGGTCGTTGTGGTGCTTGTACTTGTGCTGACTGC |
| | | TCTGTGTCTGAGCGACGGAAAACCAGTCTCCCTCAGCTACAGGTGCCCATGCCG |
| | | ATTCTTCGAATCTCATGTGGCCCGGGCCAATGTGAAGCACTTGAAAATCCTGAA |
| | | TACACCCAACTGCGCGTTGCAGATCGTGGCCCGCCTGAAAAATAATAATAGGCA |
| | | GGTATGTATAGATCCAAAGCTTAAGTGGATCCAGGAGTATCTGGAAAAGGCTCT |
| | | CAATAAACGTGCAAAGCGTGCACCGGTGAAACAGGGAAGCGGAGCTACTAACTT |
| | | CAGCCTGCTGAAGCAGGCTGGAGACGTGGAGGAGAACCCTGGACCTATGAATTT |
| | | TCTGCTCTCTTGGGTGCACTGGTCACTGGCACTGCTGCTGTATCTGCACCATGC |
| | | AAAATGGTCCCAAGCAGCTCCCATGGCAGAGGGAGGTGGACAGAATCATCATGA |
| | | GGTTGTCAAATTTATGGATGTCTACCAGCGGAGCTACTGCCACCCAATTGAGAC |
| | | GTTGGTAGACATTTTTCAGGAATATCCAGACGAGATTGAGTACATTTTCAAGCC |
| | | TAGCTGTGTGCCCTTGATGCGATGCGGTGGCTGTTGCAATGATGAGGGACTCGA |
| | | GTGTGTCCCCACCGAGGAAAGCAATATAACCATGCAAATCATGCGAATCAAACC |
| | | CCACCAGGGCCAGCATATCGGCGAGATGTCTTTCTTGCAACATAACAAATGCGA |
| | | GTGTCGGCCAAAGAAGGACAGGGCTCGCCAGGAAAATCCCTGTGGTCCTTGTTC |
| | | AGAGCGCAGGAAGCATCTTTTCGTCCAGGATCCGCAGACTTGTAAATGTTCATG |
| | | CAAGAATACCGATTCTAGGTGTAAGGCGAGGCAACTCGAGCTTAACGAGAGAAC |
| | | CTGTAGGTGTGACAAACCTAGAAGATAAATCGATTACGCTCCTCTACTCTTTGA |
| | | GACATCACTGGCCTATAATAAATGGGTTAATTTATGTAACAAAATTGCCTTGGC |
| | | TTGTTAACTTTATTAGACATTCTGATGTTTGCATTGTGTAAATACTGTTGTATT |
| | | GGAAAAGCGTGCCAAGATGGATTATTGTAATTCAGTGTCTTTTTTAGTAGCGTC |
| | | ACGTGCCAAACACTGTTAGTCACAGAGGGCATGAGACAGCCTGTGCTGGAACAG |
| | | CTCAGTTCATAGGGCTATGGAGATGGGGAGAAAGGGGCGCTTCTGTCAGAGACA |
| | | AGCTGTGGTCTGGGAAGGCCTTAGCACTAAAAGCACCACAATGAGAAGCAACCG |
| | | CCAGAAGCAGGGCCCGCAGGCCTTTGTTCCAGCTGCAAAGAGAAAGGAAAAAGT |
| | | GGGGAATAAGAGTTGGGGCTGCGGAGGGGGTGGGGAGCATTGTGCAGGTTCCGT |
| | | ACTTGAACAGAAAGCAGGGACCAACACAAGGAAGGCTCGAGCTGGCGGAATAGG |
| | | TTCCAATCTGTCGCGGCCGCATTACCCTGTTATCCCTAATCTCGTTTAACTATG |
| | | ACTCTCTTAAGGTAGCCAAATTCCGGAACTATAAATTGCGTTGCGCTCACTGCC |
| | | CGCTTTCCAGTCGGGAAACCTGTCGTGCCAGCTGCATAAATGAATCGGCCAACG |
| | | CGCGGGGAGAGGCGGTTTGCGTATTGGGCGCGCTTCCGCTTCCTCGCTCACTGA |
| | | CTCGCTGCGCTCGGTCGTTCGGCTGCGGCGAGCGGTATCAGCTCACTCAAAGGC |
| | | GGTAATACGGTTATCCACAGAATCAGGGGATAACGCAGGAAAGAACATGTGAGC |
| | | AAAAGGCCAGCAAAAGGCCAGGAACCGTAAAAAGGCCGCGTTGCTGGCGTTTTT |
| | | CCATAGGCTCCGCCCCCCTGACGAGCATCACAAAAATCGACGCTCAAGTCAGAG |
| | | GTGGCGAAACCCGACAGGACTATAAAGATACCAGGCGTTTCCCCCTGGAAGCTC |
| | | CCTCGTGCGCTCTCCTGTTCCGACCCTGCCGCTTACCGGATACCTGTCCGCCTT |
| | | TCTCCCTTCGGGAAGCGTGGCGCTTTCTCATAGCTCACGCTGTAGGTATCTCAG |
| | | TTCGGTGTAGGTCGTTCGCTCCAAGCTGGGCTGTGTGCACGAACCCCCCGTTCA |
| | | GCCCGACCGCTGCGCCTTATCCGGTAACTATCGTCTTGAGTCCAACCCGGTAAG |
| | | ACACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTACAC |
| | | TAGAAGAACAGTATTTGGTATCTGCGCTCTGCTGAAGCCAGTTACCTTCGGAAA |
| | | AAGAGTTGGTAGCTCTTGATCCGGCAAACAAACCACCGCTGGTAGCGGTGGTTT |
| | | TTTTGTTTGCAAGCAGCAGATTACGCGCAGAAAAAAAGGATCTCAAGAAGATCC |
| | | TTTGATCTTTTCTACGGGGTCTGACGCTCAGTGGAACGAAAACTCACGTTAAGG |
| | | GATTTTGGTCATGAGATTATCAAAAAGGATCTTCACCTAGATCCTTTTAAATTA |
| | | AAAATGAAGTTTTAAATCAATCTAAAGTATATATGAGTAAACTTGGTCTGACAT |
| | | GCGCATCTGACGCTCAGTGGAACGAAAACTCACGTTAAGGGATTTTGGTCATGC |
| | | CTCAGAAGAACTCGTCAAGAAGGCGATAGAAGGCGATGCGCTGCGAATCGGGAG |
| | | CGGCGATACCGTAAAGCACGAGGAAGCGGTCAGCCCATTCGCCGCCAAGCTCTT |
| | | CAGCAATATCACGGGTAGCCAACGCTATGTCCTGATAGCGGTCCGCCACACCCA |
| | | GCCGGCCACAGTCGATGAATCCAGAAAAGCGGCCATTTTCCACCATGATATTCG |
| | | GCAAGCAGGCATCGCCATGGGTCACGACGAGATCCTCGCCGTCGGGCATGCGCG |
| | | CCTTGAGCCTGGCGAACAGTTCGGCTGGCGCGAGCCCCTGATGCTCTTCGTCCA |
| | | GATCATCCTGATCGACAAGACCGGCTTCCATCCGAGTACGTGCTCGCTCGATGC |
| | | GATGTTTCGCTTGGTGGTCGAATGGGCAGGTAGCCGGATCAAGCGTATGCAGCC |
| | | GCCGCATTGCATCAGCCATGATGGATACTTTCTCGGCAGGAGCAAGGTGAGATG |
| | | ACAGGAGATCCTGCCCCGGCACTTCGCCCAATAGCAGCCAGTCCCTTCCCGCTT |
| | | CAGTGACAACGTCGAGCACAGCTGCGCAAGGAACGCCCGTCGTGGCCAGCCACG |
| | | ATAGCCGCGCTGCCTCGTCCTGCAGTTCATTCAGGGCACCGGACAGGTCGGTCT |
| | | TGACAAAAAGAACCGGGCGCCCCTGCGCTGACAGCCGGAACACGGCGGCATCAG |
| | | AGCAGCCGATTGTCTGTTGTGCCCAGTCATAGCCGAATAGCCTCTCCACCCAAG |
| | | CGGCCGGAGAACCTGCGTGCAATCCATCTTGTTCAATCATGCGAAACGATCCTC |
| | | ATTCATTTATCAGGGTTATTGTCTCATGAGCGGATACATATTTGAATGTATTTA |
| | | GGCTGAGCATCTATGTCGGGTGCGGAGAAAGAGGTAATGAAATGGCAGGCGCCT |
| | | TTTTCGTTAGATATGTAGTAAGTATCTTAATATACAGCTTTATCTGTTTTTTAA |
| | | GATACTTACTACTTTTCTTAGTGGAAACTATTAGTGGCTGTTAATTAAGCTAGT |
| | | ACTACCCAAGATTTGACAGAATGCATCGTTTGCATTCGAA |

### Results

As effectors from pXoX are expressed as single multigene mRNA, the relative expression of each effector is fixed. To determine the relative expression levels of each effector of pXoX, several relevant cell lines were transfected, and protein expression assessed by ELISA. Cells were transfected with plasmids for each individual effector (pS100A1, pSDF-1α or pVEGF165) or all three effectors (pXoX).

Accordingly, as shown in Table 15, a variety of cells transfected with pXoX triple-gene vector encoding S100A1, SDF-1α, and VEGF165, produced measurable levels of protein as detected via ELISA assays. Cell types tested included 293T cells, SV40 transformed Cardiomyocytes, iPSC-CMs (Cardiomyocytes) from healthy individual, iPSC-CMs from dilated cardiomyopathy patient, and iPSC-CMs from hypertrophy cardiomyopathy patient.

At 72 hours post-transfection, media and cell lysates were collected and analyzed separately to evaluate expression of the secreted proteins (VEGF165 and SDF-1α) or the intracellular proteins (S100A1). Table 15 below shows the protein levels of each effector compared to levels achieved by plasmids expressing each effector individually, expressed as ng/mL.

TABLE 16
Detection of Polypeptide Expression from Single-Gene and
Triple-Gene Expression Plasmids in Variety of Cell Types.

| | Plasmid | S100A1 | SDF-1α | VEGF165 |
|---|---|---|---|---|
| | Construct | [ng/mL] | [ng/mL] | [ng/mL] |
| 293T cells | Mock | 0.0 | 0.0 | 0.0 |
| | pS100A1 only | 182.6 | 0.0 | 0.0 |
| | pSDF-1α only | 0.0 | 114.3 | 0.0 |
| | pVEGF165 only | 0.0 | 0.0 | >600.0 |
| | pXoX | 1466.6 | 92.2 | >600.0 |
| SV40 | Mock | 0.0 | 0.6 | 0.4 |
| Cardiomyocytes | pS100A1 only | 57.8 | 0.1 | 0.4 |
| | pSDF-1α only | 0.0 | 20.9 | 0.4 |
| | pVEGF165 only | 0.0 | 0.2 | 386.1 |
| | pXoX | 155.3 | 7.4 | 50.0 |
| iPSC-CMs from | Mock | 0.0 | 0.3 | 0.0 |
| Healthy | pS100A1only | 1182.0 | 0.3 | 0.0 |
| individual | pSDF-1α only | 0.0 | 13.2 | 0.0 |
| | pVEGF165 only | 0.0 | 0.4 | 190.5 |
| | pXoX | 1082.4 | 4.3 | 17.3 |
| iPSC-CMs from | Mock | 0.0 | 0.1 | 0.0 |
| Dilated | pS100A1 only | 518.0 | 0.7 | 0.0 |
| Cardiomyopathy | pSDF-1α only | 0.0 | 6.0 | 0.0 |
| patient | pVEGF165 only | 0.0 | 1.0 | 191.0 |
| | pXoX | 257.0 | 1.9 | 15.6 |
| iPSC-CMs from | Mock | 23.3 | 0.1 | 0.0 |
| Hypertrophy | pS100A1 only | 361.7 | 0.1 | 0.0 |
| Cardiomyopathy | pSDF-1α only | 23.2 | 7.7 | 0.0 |
| patient | pVEGF165 only | 19.4 | 0.7 | 180.5 |
| | pXoX | 118.0 | 1.6 | 13.6 |

### Example 8: Cardiomyocyte Transfection

### SV40 Immortalized Cardiomyocytes

SV40 Immortalized Cardiomyocytes were transfected via DNAfectin Plus (ABM) with Xogenex single-gene, dual-gene (double-gene), triple-gene constructs wherein at least two genes are connected by a linker described herein, and the appropriate control constructs. A 6 well plate was coated with extracellular matrix (ABM) overnight. 24 hours later, cardiomyocytes were seeded on the 6 well plate at 225,000 cells/well into each ECM-coated well. The cells were incubated overnight at 37° C./5% CO₂. 72 hours later, media was collected from the cells into a 2 mL deep well block and stored for ELISA for detected and quantification of protein production.

iPSC Cardiomyocytes

iPSC Cardiomyocytes were transfected via GeneJammer transfection reagent (Agilent) with Xogenex single-gene, dual-gene (double-gene), triple-gene constructs and the appropriate control constructs. A 12 well plate was coated with Fibronectin (ThermoScientific) and incubated at 37° C./5% CO₂ for 1 hour. DNase was reconstituted in UltraPure water to a final concentration of 10 mg/mL Cardiomyocytes were trypsinzed using TrypLE and 1×DNase I and incubated for 5 minutes at 37° C. or until cells detached from the flask. iPSC cells were seeded at 4e5 cells/well into each Fibronectin-coated well of the 12 well plates. Plated cells were incubated at 37° C./5% CO₂ incubator for 24 hours. The following day, the cardiomyocytes were transfected using plasmid DNA at 2 μg (pXoX and controls).

### Results

Immortalized and primary cardiomyocytes transfected with a triple-gene vector encoding for S100A, SDF1, VEGF165, produced measurable levels of protein, FIG. 21.

### Example 9: Quantifying Protein Production by Transfected Cardiomyocytes

Lysates from 293T cells, SV40 CMs and iPSC CMs were diluted 1:100 in BuPH Carbonate-Bicarbonate buffer pack (0.2M) (ThermoFisher) and analyzed by ELISA assay.

### Results

S100A1, SDF-1α, and VEGF165 proteins were detected in cellular lysates of transfected 293T cells, SV40 CMs and iPSC CMs, FIG. 21.

### Example 10: Beating Rate and Contractile Duration

To evaluate efficacy of pXoX and function of S100A1 expressed by pXoX, the contraction properties of iPSC-cardiomyocytes derived from a healthy individual and a dilated cardiomyopathy patient (carrying a R173W mutation in the cardiac TNNT2 gene) transfected with pXoX or appropriate controls were measured on a Sony SI8000 Live Cell Imaging System. Cellular contraction was recorded as high-resolution and high-frame rate videos (2048*2048 pixels, 150 fps, 10 second per well). The contraction videos were further analyzed with Sony S18000C analyzer software, and the contractile parameters were pulled out for each individual well. Responsiveness of iPSC-CMs to β-adrenergic stimulation, Isoproterenol (ISO, 100 nM) was measured based on contraction properties that were recorded within 30-minutes of incubation. Three contraction parameters were evaluated during the study: beat rate, contraction rate (velocity), and contraction duration.

### Results

The beating rate and contractile duration indicate that the presence of a 2A tail at the 3′ end of a vector encoding for S100A1 does not affect the function of S100A1, FIG. 18A and FIG. 18 B. In a separate experiment, the beating rate and contractile duration shown, FIG. 19A, FIG. 19B, and FIG. 19C show the restoration of DCM contractile properties to healthy control levels by the protein produced from transfected iPSC-CM cells.

Thus, these results demonstrate that in vitro contraction properties of iPSC-cardiomyocytes derived from a dilated cardiomyopathy patient were improved with pS100A1, pS100A1-SDF-1α, and pXoX, as compared with pStuffer and non-transfection controls. (pStuffer is a plasmid with the same backbone configuration as pXoX, with the open reading frame (ORF) replaced with a non-expressing, similar-sized stuffer sequence.)

pS100A1 and pS100A1-SDF-1α displayed comparable functional output (increased beating rate, improved contractile duration), suggesting that presence of 2A tail at 3′ end of S100A1 in pS100A1-SDF-1α and pXoX does not affect S100A1 function.

pXoX was most effective in restoring contractile properties of dilated cardiomyopathy patient to levels observed in healthy individuals, suggesting effectors in pXoX are complementary.

### Example 11: Recombinase Titrations

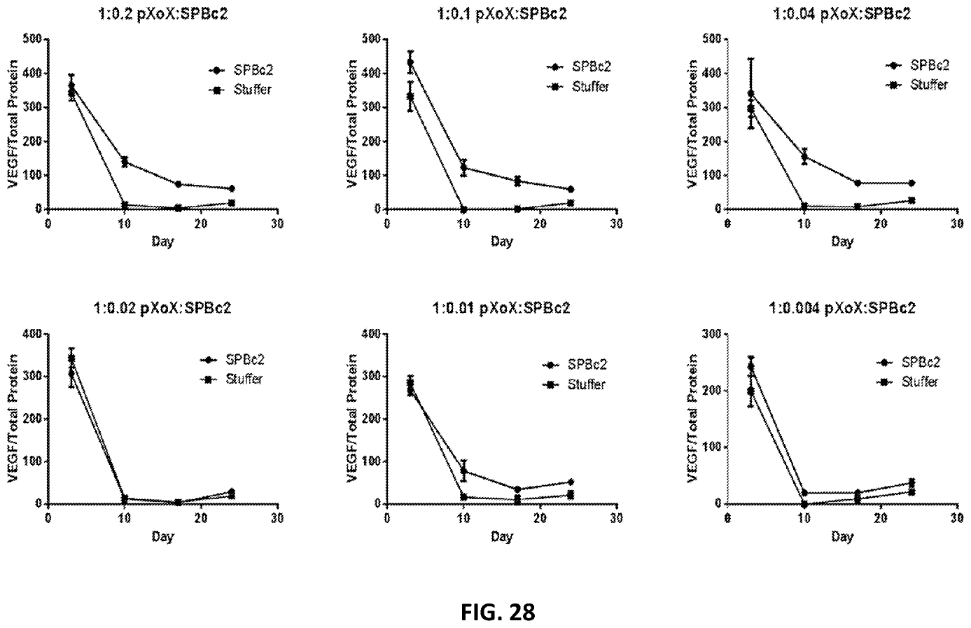

FIG. 28 shows data for expression of VEGF in a recombinase titration experiments in which ELISA analysis was performed to detect the persistence of expression of VEGF when transfected into SV40 transformed cardiomyocytes with varying ratios of the pXoX triple-gene expression vector to recombinase. On Day 3 post-transfection, there was no significant difference in VEGF expression between the SPBc2 and Stuffer groups. On Days 10, 17, and 24 post-transfection, the SPBc2 groups showed significantly more VEGF secretion than the control (Stuffer) groups when the luciferase: recombinase ratio was 1:0.2-1:0.04.

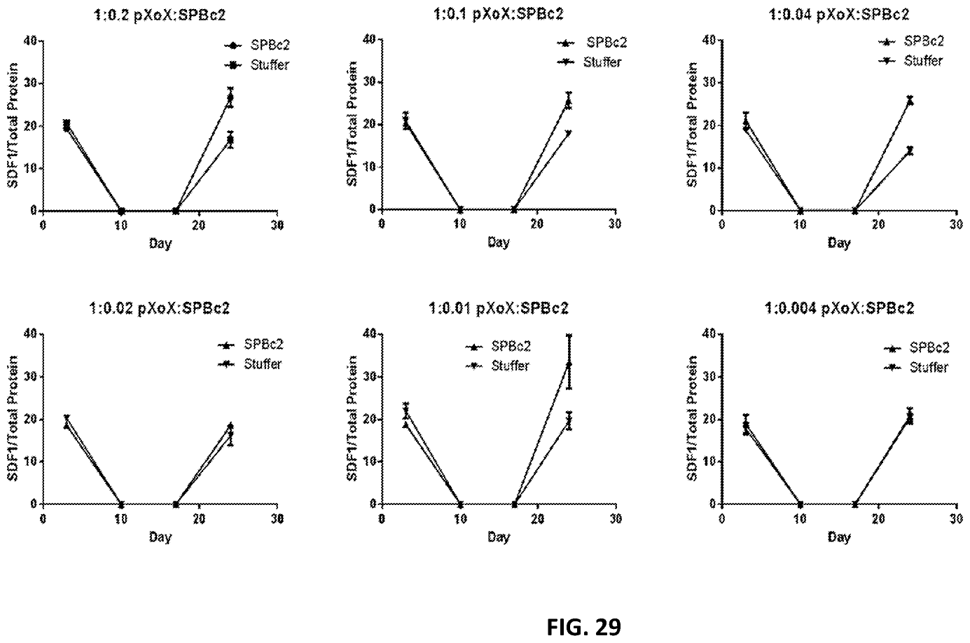

FIG. 29 shows data for expression of SDF-1 in a recombinase titration experiments in which ELISA analysis was performed to detect the persistence of expression of SDF-1α when transfected into SV40 transformed cardiomyocytes with varying ratios of the pXoX triple-gene expression vector to recombinase. On Day 3 post-transfection, there was no significant difference in SDF1 expression between the SPBc2 and Stuffer groups. SDF1 protein levels were below the limit of detection Days 10 and 17 post-transfection. SDF1 protein levels were detected Day 24 post-transfection at which point, the SPBc2 groups showed significantly more SDF1 secretion than the control (Stuffer) groups when the luciferase: recombinase ratio was 1:0.2-1:0.04.

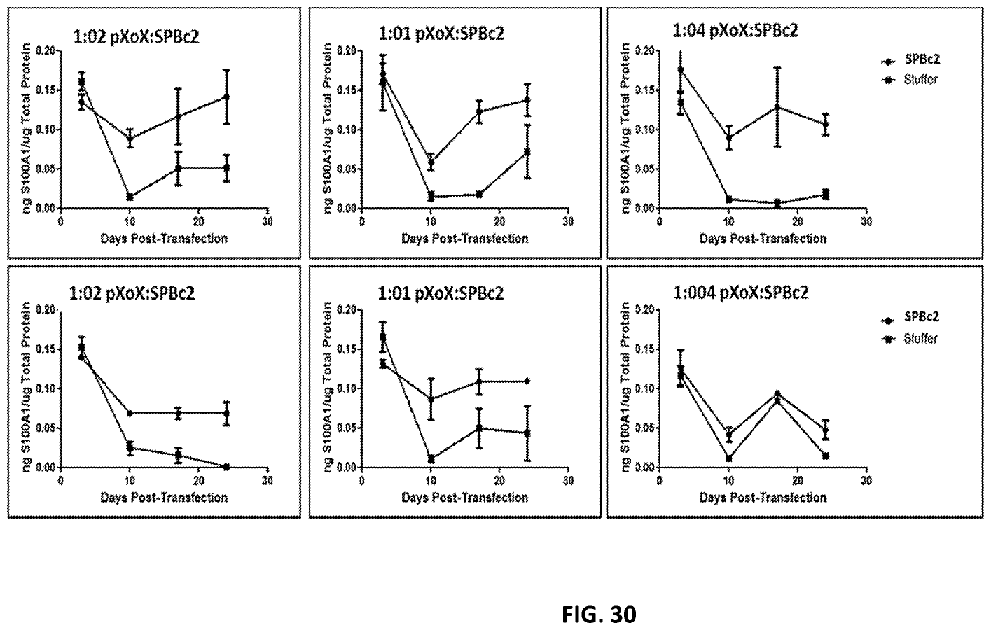

FIG. 30 shows data for expression of S100A1 in cardiomyocytes transfected with and without recombinase. On Day 3 post-transfection, there was no significant difference in S100A1 expression between the SPBc2 and Stuffer groups. On Days 10, 17, and 24 post-transfection, the SPBc2 groups showed significantly more S100A1 secretion than the control (Stuffer) groups.

### Example 12: Plasmid Backbone Expression Study in Rats

pXoX has a standard plasmid backbone with a kanamycin resistance gene. Additionally, expression of the effector proteins is driven by the hybrid CAG promoter and utilizes a synthetic 3′UTR/polyA tail. To verify the ability of this backbone configuration to maintain gene expression in the heart over time, the effector ORF in pXoX was replaced with a luciferase (fLuc) reporter gene (pCAG-fLuc) and was evaluated by live animal imaging for duration of luciferase expression after plasmid injection into the heart. A similar plasmid with the CMV promoter and a non-expressing stuffer sequence (pCMV-stuffer) was used as a control for plasmid size and background fLuc expression.

For this expression study, plasmid DNA (250 μg) was injected into the left ventricular wall of 12-14 week-old female Sprague Dawley rats (n=5 per group). Rats were monitored over time and luciferase expression levels were measured using a live in vivo imaging system (IVIS). Luciferase activity in rats injected with pCAG-fLuc were initially 2-logs higher than background levels seen in rats injected with the control pCMV-fLuc construct. Expression levels gradually dropped over 28 days after dosing as expected with transiently expressing plasmids. These findings indicate that this plasmid backbone is capable of initiating protein expression which gradually diminishes to near control levels around Day 28. Data not shown.

### Example 13: Delivery Method—Retrograde Infusion Via the Coronary Sinus in Pigs

Retrograde infusion via the coronary sinus is the proposed method of delivery for non-viral plasmid constructs (such as, but not limited to, pXoX) to the heart. The technical complexity of this delivery method precludes its assessment in small animal models such as mice and rats. As such, pigs are proposed for use in pharmacology and toxicology/biodistribution studies. To test the feasibility of this method, a pilot study was performed in pigs as test subjects. Using standard interventional techniques, a balloon catheter (Cook Regentec) was placed over the wire into the coronary sinus. The balloon was inflated, occlusion was confirmed, and infusion of the luciferase reporter plasmid followed. Three different doses (40 mg, 80 mg, and 120 mg) were tested with a plasmid concentration of 1 mg/mL. Total of 9 animals were studied, providing for 3 replicates for each dose. In each animal infusion occurred in 10 mL increments until all volume was administered. The infusion was at 10 mL/min and post infusion balloon occlusion was for 10 minutes.

Approximately 24 hours following plasmid infusion, the great cardiac vein was catheterized and a D-Luciferin substrate solution (approximately 0.125 mg/ml concentration) was infused into the coronary venous system. Animals were then euthanized and hearts explanted. The explanted hearts were subsequently evaluated for bioluminescence via CCD camera counts using the IVIS® Lumina imaging system. Bioluminescent images showing luciferase radiance intensity and distribution at 24 hour post injection of isolated hearts is depicted in FIG. 9, indicating plasmid mediated luciferase expression by the heart cells occurs within 24 hours after dosing.

Further biodistribution of the plasmid mediated luciferase expression was studied in heart and lung tissue sections. RT-PCR data further confirmed luciferase expression only in heart left ventricle in pigs in the 120 mg treatment group (data not shown). No animals died before planned sacrifice. Accordingly, this study illustrated that delivery of non-viral plasmids such as pXoX is safe, practical and results in limited systemic distribution of the plasmid.

### Example 14: In Vivo Cardiomyocytes Regeneration by pXoX in Adriamycin-Induced Cardiomyopathy Using Ultrasound Targeted Microbubble Destruction (UTMD) Method

Adriamycin induced cardiomyopathy in rats is a well established model for congestive heart failure (CHF). Ultrasound targeted microbubble destruction (UTMD) method was used to deliver pXoX (which expresses biologically active SDF1α, S100A1, and VEGF) to rat hearts in adriamycin-induced cardiomyopathy. A total of 16 rats with established adriamycin-induced cardiomyopathy were divided into 2 groups of eight. Each group was treated with one of the 2 plasmids: (1) pStuffer, or (2) pXoX. pStuffer is a negative control plasmid with the same backbone configuration as pXoX, with the open reading frame (ORF) replaced with a non-expressing, similar-sized “stuffer” sequence. Plasmid-containing lipid-stabilized microbubbles were prepared in a solution of 1,2-dipalmitoylsn-glycero-3-phosphatidylcholine 2.5 mg/ml, 1,2-dipalmitoyl-sn-glycero-3-phosphatidylethanolamine 0.5 mg/ml, and 10% glycerol mixed with 2 mg of plasmid dissolved in 50 ml of lipofectamine 3000 (Invitrogen, Carlsbad, CA). Aliquots of 0.5 ml of this phospholipid-plasmid solution were placed in 1.5 ml clear vials; the remaining head space was filled with the perfluoropropane gas (Air Products, Inc, Allentown, PA). Each vial was incubated at 4° C. for 30 min and then mechanically shaken for 30 s by a dental amalgamator (Vialmix™, Bristol-Myers Squibb Medical Imaging N. Billerica, MA). Echocardiographic measurements of fractional shortening, LV posterior wall thickness and interventricular septal end diastole and end systole (IVSd) were made from digital images acquired with a 12 MHz broadband transducer (S12 probe, Philips Ultrasound, Bothell, WA) in M-mode under 2D parasternal short axis of the left ventricle view.

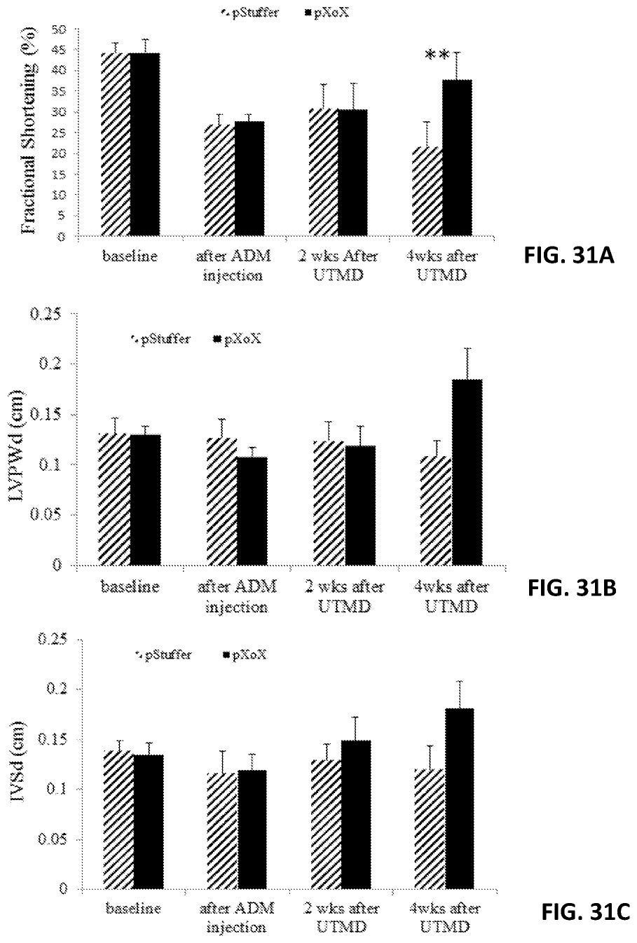

Results of echocardiographic measurement of cardiac structure and function demonstrate that UTMD-pXoX gene therapy restores fractional shortening index, LV posterior wall diameter, and IVSd, FIG. 31A, FIG. 31B, and FIG. 31C. Values are presented as mean±SEM. **P<0.001 vs pStuffer. Results show that echocardiographic measurement demonstrated reversal of established ADM cardiomyopathy after a single UTMD-pXoX treatment, thereby indicating successful in vivo treatment of cardiomyopathy in a congestive heart failure model by administration of the (triple-gene with linkers) pXoX construct which expresses biologically active SDF1α, S100A1, and VEGF.

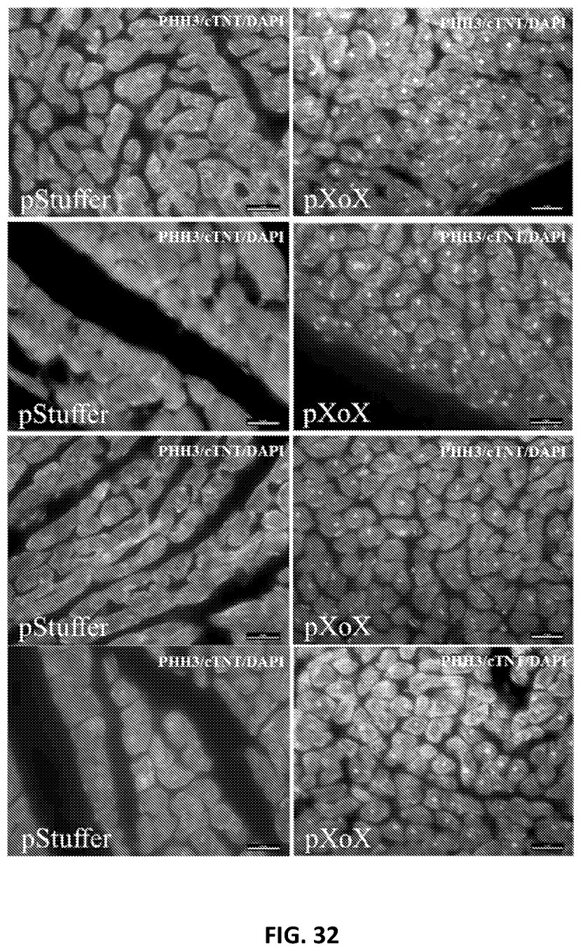

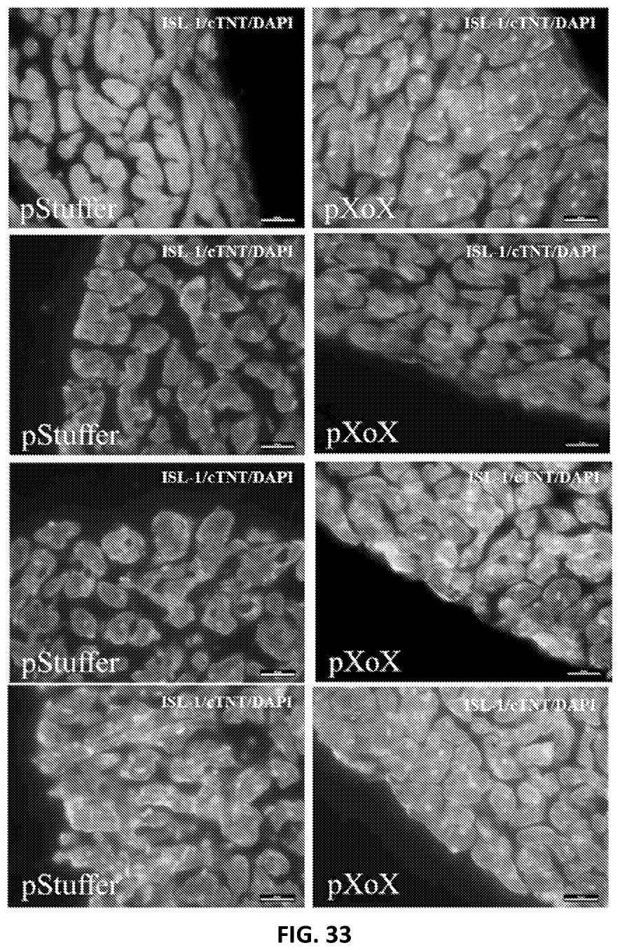

Results of nuclear localization of PHH3 (phospho-histone H3 (Ser10)) Mitotic marker signal in cardiac cells of rats receiving the pStuffer or pXoX plasmid are shown in FIG. 32 and demonstrate cardiomyocyte regeneration. PHH3 Mitotic marker confirmed proliferation of adult rat cardiac muscle cells with established heart failure using UTMD-pXoX. Negative results of ISL-1, a marker of early cardiac progenitor cells confirms progenitor cells are not involved in regeneration in the same rats, FIG. 33. Negative results of ISL-1 confirmed cardiomyocytes regeneration is truly a result of UTMD-pXoX gene therapy.

These results indicate that administration of the pXoX construct which expresses biologically active SDF1α, S100A1, and VEGF is useful in cardiomyocyte regeneration and successful treatment of cardiomyopathy in a congestive heart failure models. The results also indicate that Ultrasound targeted microbubble destruction (UTMD) is a successful method of administering the vectors and gene constructs described herein, for instance including multi-gene constructs such as pXoX for treatment of cardiac conditions and other pathologies.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

[1] J. L. Hellawell and K. B. Marguiles, “Myocardial reverse remodeling,” Cardiovasc Ther, vol. 30, no. 3, pp. 172-181, 2012.
[2] D. Rohde, J. Ritterhoff, M. Voelkers, H. Katus, T. G. Parker and P. Most, “S100A1: a multifaceted therapeutic target in cardiovascular disease,” J Cardiovasc Transl Res, vol. 3, no. 5, pp. 525-537, 2010.
[3] S. T. Pleger, P. Most, M. Boucher and S. Soltys, “Stable myocardial-specific AAV6-S100A1 gene therapy results in chronic functional heart failure rescue,” Circulation, vol. 115, no. 19, pp. 2506-2515, 2007.
[4] A. Remppis, T. Greten, B. Schafer and P. Hunziker, “Altered expression of the Ca 2+-binding protein S100A1 in human cardiomyopathy,” Biochim Biophys Acta, vol. 1313, no. 3, pp. 253-257, 1996.
[5] D. Rohde, H. Brinks, J. Ritterhoff, G. Qui, S. Ren and P. Most, “S100A1 gene therapy for heart failure: a novel strategy on the verge of clinical trials,” J Mol Cell Cardiol, vol. 50, no. 5, pp. 777-784, 2011.
[6] C. Weber, I. Neacsu, B. Krautz, P. Schlegel, S. Sauer, P. Raake, J. Ritterhoff, A. Jungmann, A. B. Remppis, M. Stangassinger, W. J. Koch, H. A. Katus, O. J. Muller, P. Most and S. T. Pleger, “Therapeutic safety of high myocardial expression levels of the molecular inotrope S100A1 in a preclinical heart failure model,” Gene Ther, vol. 21, pp. 131-138, 2014.
[7] M. S. Penn, J. Pastore, T. Miller and R. Aras, “SDF-1 in myocardial repair,” Gene Ther, vol. 19, no. 6, pp. 583-587, 2012.
[8] M. S. Penn, F. O. Mendelsohn, G. L. Schaer, W. Sherman, M. Farr, J. Pastore, D. Rouy, R. Clemens, R. Aras and D. W. Losordo, “An open-label dose escalation study to evaluate the safety of administration of nonviral stromal cell-derived factor-1 plasmid to treat symptomatic ischemic heart failure,” Circ Res, vol. 112, no. 5, pp. 816-825, 2013.
[9] E. S. Chung, L. Miller, A. N. Patel, R. D. Anderson, F. O. Mendelsohn, J. Traverse, K. H. Silver, J. Shin, G. Ewald, M. J. Farr and S. Anwaruddin, “Changes in ventricular remodelling and clinical status during the year following a single administration of stromal cell-derived factor-1 non-viral gene therapy in chronic ischaemic heart failure patients: the STOP-HF randomized Phase II trial,” Eur Heart J, vol. 36, pp. 2228-2238, 2015.
[10] Z. Taimeh, J. Loughran, E. J. Birks and R. Bolli, “Vascular endothelial growth factor in heart failure,” Nat Rev Cardiol, vol. 10, no. 9, pp. 519-530, 2013.
[11] J. X. Yu, X. F. Huang, W. M. Lv, C. S. Ye, X. Z. Peng, H. Zhang, L. B. Xiao and S. M. Wang, “Combination of stromal-derived factor-1α and vascular endothelial growth factor gene-modified endothelial progenitor cells is more effective for ischemic neovascularization,” J Vase Surg, vol. 50, no. 3, pp. 608-616, 2009.
[12] E. Perin, G. V. Silva, J. A. Assad, D. Vela, L. M. Buja, A. L. Sousa, S. Litovsky, J. Lin, W. K. Vaughn, S. Coulter and M. R. Fernandes, “Comparison of intracoronary and transendocardial delivery of allogeneic mesenchymal cells in a canine model of acute myocardial infarction,” J Mol Cell Cardiol, vol. 44, no. 3, pp. 486-95, 2008.
[13] S. Robinson, P. W. Cho, H. I. Levitsky, J. L. Olson, R. H. Hruban, M. A. Acker and P. D. Kessler, “Arterial delivery of genetically labelled skeletal myoblasts to the murine heart: long-term survival and phenotypic modification of implanted myoblasts,” Cell Transplant, vol. 5, no. 1, pp. 77-91, 1996.
[14] K. Suzuki, N. J. Brand, R. T. Smolenski, J. Jayakumar, B. Murtuza and M. H. Yacoub, “Development of a novel method for cell transplantation through the coronary artery,” Circulation, vol. 102, no. 19 Suppl 3, pp. III359-64, 2000.
[15] K. Suzuki, B. Murtuza, N. Suzuki, R. T. Smolenski and M. H. Yacoub, “Intracoronary infusion of skeletal myoblasts improves cardiac function in doxorubicin-induced heart failure,” Circulation, vol. 104, no. 12 Suppl 1, pp. I213-7, 2001.
[16] P. Musialek, L. Tekieli, M. Kostkiewicz, M. Majka, W. Szot, Z. Walter and M. Olszowska, “Randomized transcoronary delivery of CD34(+) cells with perfusion versus stop-flow method in patients with recent myocardial infarction: Early cardiac retention of (m)Tc-labeled cells activity,” J Nuclear Cardiol, vol. 18, no. 1, pp. 104-16, 2011.
[17] P. W. Musialek, A. B. Tracz, K. Skotnicki, P. Zmudka, Z. Pieniazek and W. M. Szostek, “Transcoronary stem cell delivery using physiological endothelium-targeting perfusion technique: the rationale and a pilot study involving a comparison with conventional over-the-wire balloon coronary occlusions in patients after recent myocardial infarcti,” Kardiol Pol, vol. 64, no. 5, pp. 489-98, 2006.
[18] L. Noyez, J. A. van Son, J. T. Van Der Werf, J. Knape, J. Gimbrere, W. N. van Asten, L. K. Lacquet and W. Flameng, “Retrograde versus antegrade delivery of cardioplegic solution in myocardial revascularization. A clinical trial in patients with three-vessel coronary artery disease who underwent myocardial revascularization with extensive use of the internal mammary art,” J Thorac Cardiovasc Surg, vol. 105, no. 5, pp. 854-63, 1993.
[19] P. Boekstegers, W. Giehrl, G. von Degenfeld and G. Steinbeck, “Selective suction and pressure-regulated retroinfusion: an effective and safe approach to retrograde protection against myocardial ischemia in patients undergoing normal and high risk percutaneous transluminal coronary angioplasty,” J Am Coll Cardiol, vol. 31, no. 7, pp. 1525-33, 1998.
[20] T. Pohl, W. Giehrl, B. Reichart, C. Kupatt, P. Raake, S. Paul, H. Reichenspurner, G. Steinbeck and P. Boekstegers, “Retroinfusion-supported stenting in high-risk patients for percutaneous intervention and bypass surgery: results of the prospective randomized myoprotect I study,” Cath Cardiovasc Intervent, vol. 62, no. 3, pp. 323-30, 2004.
[21] R. Incorvati, S. G. Tauberg, M. J. Pecora, R. S. Macherey, S. B. Dianzumba, B. C. Donohue and M. W. Krucoff, “Clinical applications of coronary sinus retroperfusion during high risk percutaneous transluminal coronary angioplasty,” J Am Coll Cardiol, vol. 22, no. 1, pp. 127-34, 1993.
[22] Z. a. G. M. Lokmic, “Visualisation and stereological assessment of blood and lymphatic vessels,” Histolog Histopathol, vol. 26, no. 6, pp. 781-96, 2011.
[23] D. Rohde, W. Schliiter-Wigger, V. Mielke, P. von den Driesch, B. von Gaudecker and W. Sterry, “Infiltration of both T cells and neutrophils in the skin is accompanied by the expression of endothelial leukocyte adhesion molecule-1 (ELAM-1): an immunohistochemical and ultrastructural study,” J Invest Dermatol, vol. 98, no. 5, pp. 794-9, 1992.
[24] S. Anderson, R. Shiner, M. D. Brown and O. Hudlicka, “ICAM-1 expression and leukocyte behavior in the microcirculation of chronically ischemic rat skeletal muscles,” Microvasc Res, vol. 71, no. 3, pp. 205-11, 2006.
[25] H. Habazettl, C. Kupatt, S. Zahler, B. F. Becker and K. Messmer, “Selectins and beta 2-integrins mediate post-ischaemic venular adhesion of polymorphonuclear leukocytes, but not capillary plugging, in isolated hearts,” Pflugers Arch, vol. 438, no. 4, pp. 479-85, 1999.
[26] J. Lesley, I. Gil, D. J. Mahoney, M. R. Cordell, M. S. Rugg, R. Hyman, A. J. Day and K. Mikecz, “TSG-6 modulates the interaction between hyaluronan and cell surface CD44,” J Biol Chem, vol. 279, no. 24, pp. 25745-54, 2004.
[27] A. N. Patel, S. Mittal, G. Turan, A. A. Winters, T. D. Henry, H. Ince and N. Trehan, “REVIVE Trial: Retrograde Delivery of Autologous Bone Marrow in Patients With Heart Failure,” Stem Cells Transl Med, vol. 4, pp. 1021-1027, 2015.
[28] R. E. Henschler, E. Deak and E. Seifried, “Homing of Mesenchymal Stem Cells,” Infusionsther Transfusionsmed, vol. 35, no. 4, pp. 306-312, 2008.
[29] B. Ruster, S. Göttig, R. J. Ludwig, R. Bistrian, S. Miller, E. Seifried, J. Gille and R. Henschler, “Mesenchymal stem cells display coordinated rolling and adhesion behavior on endothelial cells,” Blood, vol. 108, no. 12, pp. 3938-44, 2006.
[30] C. Hart, D. Drewel, G. Mueller, J. Grassinger, M. Zaiss, L. A. Kunz-Schughart, R. Andreesen, A. Reichle, E. Holler and B. Hennemann, “Expression and function of homing-essential molecules and enhanced in vivo homing ability of human peripheral blood-derived hematopoietic progenitor cells after stimulation with stem cell factor,” Stem Cells, vol. 22, no. 4, pp. 580-9, 2004.
[31] C. Rampon, N. Weiss, C. Deboux, N. Chaverot, F. Miller, D. Buchet, H. Tricoire-Leignel, S. Cazaubon, B.-V. Evercooren and P.-O. Couraud, “Molecular mechanism of systemic delivery of neural precursor cells to the brain: assembly of brain endothelial apical cups and control of transmigration by CD44,” Stem Cells, vol. 26, no. 7, pp. 1673-82, 2008.
[32] E. Bachrach, A. L. Perez, Y. H. Choi, B. M. Illigens, S. J. Jun, P. D. Nido, F. X. McGowan, S. Li, A. Flint, J. Chamberlain and L. M. Kunkel, “Muscle engraftment of myogenic progenitor cells following intraarterial transplantation,” Muscle Nerve, vol. 34, no. 1, pp. 44-52, 2006.
[33] P. Boekstegers, G. Von Degenfeld, D. Giehrl, R. Heinrich, C. Hullin, G. Kupatt, G. Steinbeck, G. Baretton, G. Middeler, H. Katus and W. M. Franz, “Myocardial gene transfer by selective pressure-regulated retroinfusion of coronary veins,” Gene Ther, vol. 7, no. 3, pp. 232-40, 2000.
[34] S. Alino, M. José Herrero, V. Bodi, I. Noguera, L. Mainar, F. Dasi, A. Sempere, M. Sanchez, A. Diaz, L. Sabater and S. Lledo, “Naked DNA delivery to whole pig cardiac tissue by coronary sinus retrograde injection employing non-invasive catheterization,” J Gene Med, vol. 12, no. 11, pp. 920-6, 2010.
[35] E. Youssef, P. Zhang, P. I. Rogers, P. Tremble, J. Rokovich, B. H. Johnstone, K. L. March and D. Hou, “Enhancing myocardial plasmid expression by retrograde coronary venous delivery,” Cath Cardiovasc Intervent, vol. 65, no. 4, pp. 528-34, 2005.
[36] P. Raake, G. von Degenfeld, R. Hinkel, R. Vachenauer, T. Sandner, S. Beller, M. Andrees, C. Kupatt, G. Schuler and P. Boekstegers, “Myocardial gene transfer by selective pressure-regulated retroinfusion of coronary veins: comparison with surgical and percutaneous intramyocardial gene delivery,” J Am Coll Cardiol, vol. 44, no. 5, pp. 1124-9, 2004.
[37] G. von Degenfeld, P. Raake, C. Kupatt, C. Leberhz, R. Hinkel, F. J. Gildehaus, W. Miinzing, A. Kranz, J. Waltenberger, M. Simoes and M. Schwaiger, “Selective pressure-regulated retroinfusion of fibroblast growth factor-2 into the coronary vein enhances regional myocardial blood flow and function in pigs with chronic myocardial ischemia,” J Am Coll Cardiol, vol. 42, no. 6, pp. 1120-8, 2003.
[38] K. Suzuki, B. Murtuza, S. Fukushima, R. T. Smolenski, A. Varela-Carver, S. R. Coppen and M. H. Yacoub, “Targeted cell delivery into infarcted rat hearts by retrograde intracoronary infusion: distribution, dynamics, and influence on cardiac function,” Circulation, vol. 110, no. 11 Suppl 1, pp. II225-30, 2004.
[39] J. George, J. Goldberg, M. Joseph, N. Abdulhameed, J. Crist, H. Das and V. J. Pompili, “Transvenous intramyocardial cellular delivery increases retention in comparison to intracoronary delivery in a porcine model of acute myocardial infarction,” J Interv Cardiol, vol. 21, no. 5, pp. 424-31, 2008.
[40] C. Thompson, B. A. Nasseri, J. Makower, S. Houser, M. McGarry, T. Lamson, I. Pomerantseva, J. Y. Chang, H. K. Gold, J. P. Vacanti and S. N. Oesterle, “Percutaneous transvenous cellular cardiomyoplasty: A novel nonsurgical approach for myocardial cell transplantation,” J Am Coll Cardiol, vol. 41, no. 11, pp. 1964-71, 2003.
[41] P. W. Raake, R. Hinkel, S. Miller, S. Delker, R. Kreuzpointner, C. Kupatt, H. A. Katus, J. A. Kleinschmidt, P. Boekstegers and O. J. Miller, “Cardio-specific long-term gene expression in a porcine model after selective pressure-regulated retroinfusion of adeno-associated viral (AAV) vectors,” Gene Ther, vol. 15, no. 1, pp. 12-7, 2008.
[42] J. Tuma, R. Fernindez-Vina, A. Carrasco, J. Castillo, C. Cruz, A. Carrillo, J. Ercilla, C. Yarleque, J. Cunza, T. D. Henry and A. N. Patel, “Safety and feasibility of percutaneous retrograde coronary sinus delivery of autologous bone marrow mononuclear cell transplantation in patients with chronic refractory angina,” J Transl Med, vol. 9, p. 183, 2011.
[43] N. T. Wright, K. M. Varney, K. C. Ellis, J. Markowitz, R. K. Gitti, D. B. Zimmer and D. J. Weber, “The three-dimensional solution structure of Ca(2+)-bound S100A1 as determined by NMR spectroscopy,” J Mol Biol, vol. 353, no. 2, pp. 410-426, 2005.
[44] R. Donato, “Functional roles of S100 proteins, calcium-binding proteins of the EF-hand type,” Biochim Biophys Acta, vol. 1450, no. 3, pp. 191-231, 1999.
[45] D. B. Zimmer, P. Wright Sadosky and D. J. Weber, “Molecular mechanisms of S100-target protein interactions,” Microsc Res Tech, vol. 60, no. 6, pp. 552-559, 2003.
[46] L. Hove-Madsen and D. M. Bers, “Sarcoplasmic reticulum Ca2+ uptake and thapsigargin sensitivity in permeabilized rabbit and rat ventricular myocytes,” Circ Res, vol. 73, no. 5, pp. 820-828, 1993.
[47] J. James, Y. Zhang, K. Wright, S. Witt and E. Glascock, “Transgenic rabbits expressing mutant essential light chain do not develop hypertrophic cardiomyopathy,” J Mol Cell Cardiol, vol. 34, no. 7, pp. 872-882, 2002.
[48] J. Woda, S. J. Fisher, J. Pastore and A. N. Patel, “Coronary Sinus Delivery of SDF-1 Plasmid for the Treatment of Heart Failure,” American Society of Gene and Cell Therapy, p. poster presentation, 2014.
[49] M. Hedman, J. Hartikainen, M. Syvinne, J. Stjernvall, A. Hedman, A. Kiveli, E. Vanninen, H. Mussalo, E. Kauppila, S. Simula and O. Nirvinen, “Safety and Feasibility of Catheter-Based Local Intracoronary Vascular Endothelial Growth Factor Gene Transfer in the Prevention of Postangioplasty and In-Stent Restenosis and in the Treatment of Chronic Myocardial Ischemia. Phase II Results of the Kuopio,” Circulation, vol. 107, pp. 2677-2683, 2003.
[50] M. G. Katz, A. S. Fargnoli, A. P. Kendle, R. J. Hajjar and C. R. Bridges, “Gene Therapy in Cardiac Surgery: Clinical Trials, Challenges and Perspectives,” Ann Thorac Surg, vol. 101, pp. 2407-2416, 2016.
[51] P. Kolsut, M. Malecki, P. Zelazny, B. Teresiinska, P. Firek, P. Janik and Z. Religa, “Gene therapy of coronary artery disease with phvegf165-early outcome,” Kardiol Pol, vol. 59, pp. 373-384, 2003.
[52] M. Ruel, R. S. Beanlands, M. Lortie, V. Chan, N. Camack, E. J. Suuronen, F. D. Rubens, J. N. DaSilva, F. W. Sellke, D. J. Stewart and T. G. Mesana, “Concomitant treatment with oral L-arginine improves the efficacy of surgical angiogenesis in patients with severe diffuse coronary artery disease: the Endothelial Modulation in Angiogenic Therapy randomized controlled trial,” J Thorac Cardiovasc Surg, vol. 135, pp. 762-770, 2008.
[53] S. T. Pleger, C. Shan, J. Ksienzyk, R. Bekeredjian, P. Boekstegers, R. Hinkel, S. Schinkel, B. Leuchs, J. Ludwig, G. Qiu and C. Weber, “Cardiac AAV9-S100A1 gene therapy rescues post-ischemic heart failure in a preclinical large animal model,” Sci Transl Med, vol. 3, no. 92, pp. 92ra64-92ra64, 2011.
[54] R. J. Lee, M. L. Springer, W. E. Blanco-Bose, R. Shaw, P. C. Ursell and H. M. Blau, “VEGF Gene Delivery to Myocardium: Deleterious Effects of Unregulated Expression,” Circulation, vol. 102, pp. 898-901, 2000.
[55] J. J. Lopez, R. Laham, A. Stamler, J. D. Pearlman, S. Bunting, A. Kaplan, J. P. Carrozza, F. W. Sellke and M. Simons, “VEGF administration in chronic myocardial ischemia in pigs,” Cardiovasc Res, pp. 272-281, 1998.
[56] M. Katz, A. Fargnoli, R. Williams and C. Bridges, “Gene Therapy Delivery Systems for Enhancing Viral and Nonviral Vectors for Cardiac Diseases: and Nonviral Vectors for Cardiac Diseases: Current Concepts and Future Applications,” Human Gene Ther, vol. 24, pp. 914-927, 2013.
[57] Brinks H, D. Rohde, M. Voelkers, G. Qiu and S. Pleger, “S100A1 genetically targeted therapy reverses dysfunction of human failing cardiomyocytes,” J Am Coll Cardiol, vol. 58, no. 9, pp. 966-973, 2011.
[58] F. J. Giordano, “Retrograde coronary perfusion: a superior route to deliver therapeutics to the heart?,” J Am Coll Cardiol, vol. 42, no. 6, pp. 1129-1131, 2003.
[59] Y. Wang, F. Wang, R. Wang, P. Zhao and Q. Xia, “2A self-cleaving peptide-based multi-gene expression system in the silkworm Bombyx mori,” Sci Rep, vol. 5, no. 16273, pp. 1-10, 2015.
[60] M. C. Scimia, A. M. Gumpert and W. J. Koch, “Cardiovascular gene therapy for myocardial infarction,” Expert Opin Biol Ther, vol. 14, no. 2, pp. 183-195, 2014.
[61] I. Marenholz, C. W. Heizmann, G. Fritz, “S100 proteins in mouse and man: from evolution to function and pathology (including an update of the nomenclature),” Biochem Biophys Res Commun. vol. 322, no. 4, pp. 1111-22, 2004.
[62] T. Nagasawa, “CXCL12/SDF-1 and CXCR4,” Front. Immunol, vol. 6, pp. 301, 2015.
[63] David I. R. Holmes, I. Zachary, “The Vascular Endothelial Growth Factor (VEGF) Family: Angiogenic Factors in Health and Disease.” Genome Biol. Vol. 6, no. 2, pp. 209, (2005).
[64] S. Chen, J. Chen, P Huang, X L Meng, Clayton, J S Shen, P A Graybum, “Myocardial regeneration in adriamycin cardiomyopathy by nuclear expression of GLP1 using ultrasound targeted microbubble destruction.” Biochem Biophys Res Commun. Vol. 458, no. 4, pp. 823-9, 2015.
[35] C. R. Mayer, N. A. Geis, H. A. Katus, R. Bekeredjian, “Ultrasound targeted microbubble destruction for drug and gene delivery.” Expert Opin Drug Deliv., vol. 5, no. 10, pp. 1121-38, 2008.

1-164. (canceled)

165. A polynucleotide encoding a furin-cleavable polypeptide linked to a 2A self-cleaving peptide sequence by a polypeptide linker comprising at least three hydrophobic amino acids.

166. The polynucleotide of claim 165, wherein each of the hydrophobic amino acids is each independently glycine, alanine, valine, leucine, isoleucine, proline, phenylalanine, methionine, or tryptophan.

167. The polynucleotide of claim 166, wherein the polypeptide linker comprises an amino acid sequence that is at least about 90% identical to any one of SEQ ID NOs: 42-101.

168. The polynucleotide of claim 167, wherein the polypeptide linker comprises an amino acid sequence that is at least about 95% identical any one of SEQ ID NOs: 42-101.

169. The polynucleotide of claim 168, wherein the polypeptide linker comprises an amino acid sequence that is at least about 99% identical to any one of SEQ ID NOs: 42-101.

170. The polynucleotide of claim 169, wherein the polypeptide linker comprises an amino acid sequence of any one of SEQ ID NOs: 42-101.

171. The polynucleotide of claim 167, wherein the polypeptide linker comprises an amino acid sequence that is at least about 90% identical to the sequence of SEQ ID NO: 42.

172. The polynucleotide of claim 166, comprising an amino acid sequence that is at least about 80% identical to the sequence of SEQ ID NO: 40.

173. The polynucleotide of claim 165, wherein the 2A self-cleaving peptide sequence comprises a p2A, GSG-p2A, T2A, F2A, BmCPV2A, and/or BmIFV2 self-cleaving peptide sequence.

174. The polynucleotide of claim 173, wherein the 2A self-cleaving peptide sequence comprises a p2A self-cleaving peptide sequence.

175. The polynucleotide of claim 165, further encoding: (a) a calcium-binding polypeptide; (b) a cytokine, or functional fragment or variant thereof; (c) a chemokine, or functional fragment or variant thereof; and (d) an angiogenic polypeptide.

176. The polynucleotide of claim 175, wherein the calcium-binding polypeptide is a S100 polypeptide or functional fragment or variant thereof.

177. The polynucleotide of claim 176, wherein the calcium-binding polypeptide is a S100A, S100A2, S100A3, S100A4, S100A5, S100A6, S100A7, S100A8, 3100A9, S100A10, S100A11, S100A12, S100A13, S100A14, S100A15, or S100A16 polypeptide, or a functional fragment or variant of any one of the foregoing.

178. The polynucleotide of claim 175, wherein the chemokine or functional fragment or variant thereof is a SDF1, SDF-1α, or SDF-1β polypeptide or functional fragment or variant of any one of the foregoing.

179. The polynucleotide of claim 175, wherein the angiogenic polypeptide is a VEGF polypeptide or functional fragment or variant thereof.

180. The polynucleotide of claim 179, wherein the angiogenic polypeptide is VEGF121, VEGF121b, VEGF145, VEGF165, VEGF165b, VEGF189, VEGF191, or VEGF206, or a functional fragment or variant of any one of the foregoing.

181. The polynucleotide of claim 175, wherein a compound of (a) through (d) is linked to another compound selected from (a) through (d) by a linker sequence comprising the furin-cleavable polypeptide and the 2A self-cleaving peptide sequence.

182. A method of treating a disease or disorder in a subject, the method comprising administering the polynucleotide of claim 165 to a subject in need thereof.

183. A composition comprising the polynucleotide of claim 165 and a pharmaceutically-acceptable excipient.

184. A vector comprising the polynucleotide of claim 165.
